SEQUENCE LISTING (235 sequences)

SEQ ID NO: 1 - DNA, 83405 bp, Homo sapiens
  misc_feature (18828)..(18833): this region may or may not be present
aaccatcacccatactgtccctttaaaccagaactctgcttgccactctcctttcstaactcttgggcatccttcaaggctcagtatttgtcacttcgtccagaaatgtttcctgactccccaatccgagtccctaatacctgctgcatagcccaatccggcatctataaaattgtttcaattgcattcttattcatctgtgtctcctaagacactgtgatagggcaggaatggtgtcttggatatcactgatctccagtcccagtcaataaattggatttctgatttaagtctcctatgatctggatactccaagttggagcattcagagcaaagaatctgcttggagtttccttagccaattgggtaagccccaggctggtgcagtggctcacgcctgtaatctcagcactttgggctgctgagatgggagatttgcttgagcccaggaagttgaggctacagtgagccatgattgcaccactgcgctatagcctggatgacagagtgagaccctgtctgaaggaaaaaaaaaaaaaaaaagcaaattgggcaagccttagggtctgaaccatggtaaatttctgttactttgtgaggctatttggcgaccactgtgcttgtaaaatagctttagtgacagttaagctttgtcacaagcagggtctactttgaaattcagtctgcacatctgcctctaaccagccttccctgtcagagctcatagattagctgccatgaagagtcatcccaaatgtgttgggtctttcatggataggttccgaagtcattcctacacaccagagtggatggtgcaatggaagctatggcccttatcacagattcggatttgtgtagtagaaaaaaagacttgtctgggaaccaggaaattgcggttctagttccagcataatcacttaactcaatctatagttactaaacttcttgaggcctcagtgtattacctggatacactaggttacgtagcaataataaattaacaatgacctctcagtggcttcacacaacaaaggcttatctcttggtcctcgtgtgtatctattccaggtctgcgaaaaggctctgttccctctggtcactcagggacccagcctgatggaggttccatcacctagtagtttctgtggcatttctaccctccttaaccaccaggggtccaggaaaggaagagatggaggagacgtgcacatgggctttcactgccttaattcagagctgtgcttcacttcttctcagagcccattggccagaacttgtcatgtgacctcgcctaactttagctgagctgcgaaatatagaggaacaaatggaatcttcaatgacaattatagtctaggccatattgagtttccacatctgttgataaagttatgataataatagtatgatttacctcatagagatgttgggaagatgaatgcatgtgacatgattaacattgtgctggctcatagaaagtatttcacaaattctagccataataagtatattattattattattattattaagtgggaaataagttgtagcattattatcactatgtaattcttctaagagcaaaagtgactgtgcctaagctcttaaccatttgtgcctaagctcttaacctgttgagccatcctgaaggcattctgctgtacactctgctcaaggaagaagaaaagcttgttccaggaaagcttgttgttgaactgtatgcccttcccccctctacctttggtcctaccccttctgccaatcctggccatgactgcccctgtcagcaacccagggaagggtctgaggacattggaagcctccagctctttcttctcctacatgattttccagggctcagtgttcccctgatgctcccaggctgtcagtgtgagacacccctgtggcctccagagtcatgatgggcccactcaggtttcctggcagtaaggctcatatgcaaacaaaactggaaccagaaggaattataaaaatggttccactcctcaagcacactcttgtctggaaatgtttttcaactttctattctgtttggtttgtggaggttaaaaaaaaaaaaaaagagagagaacgaaaacaaacctacaggatctgattaaaagccaaaaaggctgttgtaggagaggccacacttcttataaacatacaaatgcacagcagactatctgaagtcaaggtggtctttgagcctaataattggtagcaaatttagccaagctgcttagcctgagagtgatttctgtcctctgactgctacgttggctttgcttgcttacatttggtcttccaaatcagtgccattcctttctacctccatgcctttgcctgtgctattctttcttcctggaacaccctttcttcctgtcccttgcacgccacctgttgaataaatcccactaattcttcaaggtgtagtttaaatgccactcattgtttccttctaatctttcaaggtataatcacaaggattatgtctttgtatgctattagcacctagcatggtgtctggtgttcaataaatgttgagttgaaatcccccttttcttcattaattttatcccttagtgttatgtttatggagactatcaacggggacaaagtttcaagaaatttggggaaagctgcttctgccccagagtcccttcaagaaaagccttaagtgctgaaattccctgagaagtttaagcttgtaacacttgcagtggttcccccaagaaatcaacccgaactatcttgtatcctcttaccttgctgaaattgattttcaaatgaaggcataatgttagcttcatctcatatgaggactcttcttcccaaggaagctaccttgttattatagcaactacctttatggagctttcccgattgcaaagtatatgtgcatacattatcttgtttagcactcattgtgatcatggggcttggtagagcagaggatgacattgcgtacacaactcctataatcaagggaatctggcagtctctgttatttcagcagagtcaatctaaacaaattttggatggcttgggggaaagaaactcagaatttaccagctgctataacctcccaaacagcatgaagaaaatgacactgttgttttctggaaagcaaggtttcatacccaagaattattgtagggatttggaatcccaaatcaagtattctcctgtattttaccacatatttaaactctgaggtgcttgcttccaaactggtcaatgtagcaactgaatccggggccattagccatttgttctgatccagccacgtcggctagcagggctctgaatcaatttcaaaaggacatacccataagagccttttgataggtgaggtaggggttagcttggtggccctggaagaaatgggtaactaatgttccaagagcaaatctttgactggtgggagatggaaggcggtagataaattattctcccgttttttttttttttttttttttttttgcagggggtttgaggggaaggagtctagctctgtcaccccggctggagtgcagtggcgtgatttcggctgactgcaacctccgcctcccgggttcaagcggttcccctgcctcggcctcccaagtagctgggattacaagcacgtgccaccatgcccggctaatttttttgtatttttagtagagatggggtttcagtatgttgtccaggctgatctcaaactcctgacctcaggcaacccgccagccttcgtctcccaaagtgctgggattacaggcgtgagacactgtgcccggcccctttgttttctaagcaaaataacacacacttaccaaagtttacaggaactgtatgtttagaaactctaagaattcatattctattctcaaaaccaattttcacagaaattggcttcataggagttattttattttcatcctttacgatggtcatgctgaagccagtaagactgattgcaccctaactcagagtcacacactggattaggtagttctggaattatagacaagtcttccaaaaatccatagtcagctagtcaagagaagtagacaggtaaagtatgacatattgtgaaaagttttatcagttatgaataaaatatcatggaaatagagaagatgggggaatagtttctctaggggaattgaaggaaggtttacttctttggagatgtcctttcttgaacttaccaaaaacttaccaaaacttacagcaactctgtgttttgttcacatcagtacacttagttgtagtatcattccttgttcagagcaaagcattcagtaagtattttggcctgaaaaacagctaatatatagcggcgaccactggccaggcactgtgtgctttccttgcattatgttatttaatcctcacaggagccttacgagcttcctcaggagagaaccaagagttaaagaagttaagatggtcaccacagttcctagttagaggcagacctaatcctgaactcacctctcttggattccaacccatgttctcaaccactggaccaaactcactccattaattagagaagctgtgttgctgttttgatgatcattgctggtatgattactggccacccattggtgcccttaactcattttcatagcaaataggagactgtcacagctggggtaattagaacaggacctgagaacccctttggccagatgccttctagaaataacagtgtaccggtttgctcaagttggagagcttcattctcttgaattgtatgagaattccctggctaaatgtcaagcctacaggtttacaatcttccatctgactcttgagctaactaacatctgagaatccctgcattgtttggctttggaagatatgaagaaattagacttttgtctttaggtttacatttttcgagtcctgcttttcagaagacatttccactaggttgggaatgttatacaacagctgtcacacaaagttagcagggcgcacatgaagtataaagtcaggtgaggatactgcctatgggcatggaacatcaaatagggccttaaaaaaattggacagcattctttaagaggaatcagccagcctgtggcctttgaagcagtgacaggaaagagcccagaaagccttgcttctttaagtggcacaacttgctttccttgtggtcctcaaagtcagctcaacagaagccacaaacctgaggtttatgagctggatctgtctgccaaggtatgggtggcccgcatggtattgaaacagttttgaatcatttatcacgttttaaaatcaggagtgagaagaatctggacctctggcctttcctgaggcaatgaggcaatattctgtctccttttctgaaccagcagttagtgacatttgagcagacactgtgctcagctcaaagggagcccctgccgtttcatccagttcagcactgttcctcctcaccctccactgctagtcacctggctgcttcactcatttccatgacctgcctggccactaagcgtttgcacacatgacctagagcacagctgggaagacagctgggggaatacagaaagcagggcagatggctgctagggcatggtcttgaaataaaaaccttcctgtcctacctgtttaaaagtccctctgctgacttgccagttgactctaatgtatttggaggtaacagtctaaattttgagtagctggagctgttgaagtcatctctgcaagtcttcaatagtgtgagcacaggaatggttatttgaaatatagaacactgaagcaccaggtcgagaggacatcgaaggtgaaggagccactcaccaaagatgaagcattaggaaggtctaaagtagtaaagtagaattaattcagttttagagaaactaagttaaagtgtatgtagttcctccaagtagagatgtccacctcacgacgtactttagaattagaatccaagtttaaaaccttgcccaagtgcgtgcacctaagcggcctgaatgaaagccaatgttatcttcatcatacaaatgttagctgcttttcaaactgcttttaattagctcatagggttttcacaactcttggaaatcggtgggactcgactttcacttttagacccagatacagaatgacacttatgaagtttcaggttctataatcaaattaagattacagcctggggcctaggcctctggcccattgctcttcccactgtaccactctggtaactccaagtttcaggacagtcaagagactggattctactgtctacacctaggcctttgaattcaaacacgcatggttctgctctagctaaacatcatttcactttactcactatgccaagccatcttttcaattttagcaaaagctttttatcatctgagaatcttttatttctgaacttcccccaggtactgtggcatgctgtattctaggctctcagcactttcacattaatcataattctttactagaggcatctatggcagtgagccccaaattcaagctgctttgggtttcaggataattgatgcttagtaaaatctgatttttttcttttaaaaacagcaacaaatttacactcaaatcagggtttaaaccataaaatcgcagggctttaaagctcacagtgggggccggagcgggggcaggggcaacaggcctagtcagaggctagaatgtaaaagtgggtacatgtcaggttactgaacgtcctggttttgagtttggcttctcagattgacctaggatatttctagcacctgcctacccacctagcttgtttggagcaccaaggatgtacacacatagcatgtcagtttgtatgtacttagcagttacagagatatatttatgccccaaagcttcctctgtaccagccatttgttggtttcatttttaatcttaataccttcacacataacaattatatatttattaaaatttcagatcacctagttctgttaacaaagaaggttggagacaagctagtctgtcttccattttttagtctaagacctctttcttttgaaaaattcatttggtttgtatggataactattctagataaggcaaacaaacgaagtgtttggctctattaactttttcagccaactttcccttggggaccaccaccaactgaataatgaatactcaaaaaagtacagcttataacacaacttttattagaaaagttatacataacatagcatcaactattttcaagaacaatattaaacccgataagcaacaaaaaccagactaacaaaatgtgtaacaagaaactaatgacctttctaaaatcaaacattcaattatctacaatgtctttttacaaacrgggaaaactccttggtttacaggcacatcatattgaatrtaaagctgcaatagcaattttatacaattaccactctgaagaaactgaatcattaaaacagtaattacgagttcacaaatttaaaacatttcacataattttaaattattgggtatacactgaagtctgagtttcaaaagtgatttttttttcccacaaaagtttcaacacttaagctagaactttcagtgttaactttgccctaaaaagttaagacattctgataatcataacagtcacatgatttctgatgctatctggtctgttaataataaagtctttatttggatgtatttttcttcaattaaattacaggaaactggatataggatttcgttgcaacgctattaaagttccaaaccaggagtgtgcagcactggaaaaggagatcagtactaaaacttacaataaatatcagagaagccgttagtttttacagcatcgtctgcttaaaagctaagttgaccaggtgcataatttcccatcagtctgtccttgtagtaggcagggcaatttctgttttcatgatcggaatactcaaatatatccaaacatctttttaaaactttgatttatagctcctagaaagttatgttttttaatagtcactctactctaatcaggcctagctttgctcattttggagcctcactaaaataacagatttcagtatagccaagttcatcagaaagactcaaatggaatgatttacaaaatagaacactttaaaccaggtcagtcctatctttttgtagctgaaggctatcagtcataacacaatttcgcgtacacctctgctcattatggaattacacttaaaacgaatctcaagagggtgaccattgttgtttcagataccatccctaaggagagtggttaacaggaagattgccagtgttactgatggaaagaagtgtttgtttgtttttttttcttgtcaaagacttacaccatagttttaaattaaactgtcaggcattttctcagacaggttttccttttcaatgcagtaatgaagaactaagataaaaatcatgacttttgactgccactcaacattattacatgcaccaatattgcacacatctgttctgaactgttaaaatcatcttctgagtccttggggtgctgttttctccatcagaacacaaacacaacccatctaatcagtttccctcaaagatgaaattgacaaatttaatgtactggaaaaaaatgaagaaggaaaaaggcaaagactttgtacagacaaaaatctaagttttctcaaagggttctgtgtcccctacacatgggggcaatttgtaagcactagtgaatcaaacactagctataatgcttctagctccttatataatatggaaccttggtccaggtgttgcgatgatgtcactgtacggttcttcctgtgtcagctcaatagcttgctgctttttaagaaccaagaagctgtagaactttgcggcagcttgttttctgttcgtatttcgacataactcaagcaaactgatagattcagctccagttttagcaagagcacgctgaaataaaaccaaaaaagggtaacttaatctgtataataaagtagtaattcagtgaggatgctgatacagtttgaatatttgtcccctccaaatctcatgctgaaacgtaatttcccagtgttagagatggagcctagtgaggtgtttgtgtcatggggtggggatgggggtggtggtggatcccttatcaatcactccctgtggcaatcagtgaattcttgctcgttagttaacacaagagctgcttgtttaaaagcgcctagccatctctctcatgccctctcttgccatgtgacacacctgctccccctttgccttccaccwtgactataagctttctgaggcctcaccagaagcagatgctggtgctttgcttcttgtatagtctgcagaactgtgagccaaataaacttctttataaattacccagactcaggtattcctttagagcaacacaaaacagactaacacagatgctaattttggttactggaaaaaacaaactatatattctgcttaggacaaagattgcacttttttaaatagtatgtgattctcatgttaatcaagagggtaaaaggccaaagtatgttatggtacatctaaaatccaaataagaaagtccaagaaaccagcaaaattgttctgacatgtattattaaaataaccaatttctttagacagctaatgtaatatagtagtaaaatctagcacttatagtttttaaagtaaaaaattaataaacacatgtcaactgtcctattttaagaaaccttgagcaacttgcttggcattttgcagacattcaagatatcctgtctatgtctaggacattaaagcacaaggaaaagtaacgtctgatgctacacaatgacccaataaatttaatgattcagcacattaaatttactcaacaaaaaacaagaaggtgcaggcatacaaaggagttgtgtggtatgtcaccatggttctgcttttcaggaaaaccacaagcattctcctccatgcttttccggcttactcttggcctaacagtttatgtatttaattaaaacttaaactataataaaaaaaaaaaagaaaagaaaactctccctccagaaaaaataaataaataaataaaaatgtagttttggaaaaagtttgacaaaggtatgctttctgtttccagcatctaactttttttttttcagggaacaatggcaaaagcctttgatgaagcattttcctctgagacaacagcggcaatacctgaagaccatgaagcatctgctgagtccttttgttccatcttctttcttcctgatcttgatcgccccctgatgcatcttcatcctgaataaaaatgacccccaaaaagctgacaaaacaagtccaatgaaaaatgaagtatttctacatctcctaaaagctcctaaattaccttgagattgtgtattttgaactaatagaaaactgtacttaatgctgctgagcaatcacttagcatttagagtacaaactgaagagtctgtttttcttaaattacaaggcaaagtcctctatgtgcaagcaaacatttggctgaaagtatgcttctatartgcgctataaggatgctatctacacacactatgtggtctgtggactgctgctgatcatggaggttttggttactggcccctcttaaagaacttcaggatataaatcaactgtaccactaaatatactgtttatttcaactaaatatacatacataaatattgcaacatgattttctagatgactaatgttctggcagactcttaatctcattgtggagcagtaccaaagagcctgcagactacctaagttgatttatatggtgttttaaaatgaatgggctatactccttattatatatttcacttgagcaacctagttctcagccacatgctgcctaaatgactttttaaagtccaaggtgggtgcaaggcagcagttgtgatgttcccaggctatggtgaaactatataccagccataacagaaaccacaactattttaatactcattcacaactaagtctgagataatggcacatcctcacacataggtagaagtggctctgaaactaattttggctttaaaataagatattcatattaaagataagcactattattagaattaagaacagatgctactgaaaatttatccaatccataaccttttttggtcttgaagaaatttaaacagcttcaatgctacctaaaggacaccttattttaaatttaaaggtttaagtaacatatccgatagagtatcaaacagaagaataaattctaagtcttaaacaacaacctcaatgatccagaaattaggaagaaaaagaagaaaaaagaccttcaacagtgtaaacatgccccaggaaaaagaaattgattcctaaaacacctcttactttgtaaaaagaaaaagtcatttaagccttcactcagtctaagtgttttagcaaagttatgctttaccctggtccaagtaaaagaaaaacaaaaacaaaacccagacaagtggaaaacatatggttatctgatttccaggccaccacagatacagaaaaggttatatcagaagcagacaataacagcagtaagaatcgtccactgaccacaagatagtaaaagcagctacacgcaatttagaagccaagcaagaaatcagtgtaaggacaattccatgtccttcaaaaccccacaagagagccaatgagttacagcgaagcataaaatttgatgttcctaatgatgatcaataccacgaagaatatggtaaaattttgcttatattctaacatttttaaataactctgatgctcagcatcaattaagttagccccaatatgaaaaataaagctggttggaggtttttgctaaatctggaatgaaaattattaattagtttacctcttcctcttcatcatcttctttttccttctctttrtccttctctttttctggcagaagttctaactctggtattagctgacagatatttggaggttcttctgggggaagctctacaggtggtatttccatctgctctacctgctgaggcttaaagcaatacaaataagacaatttaagatatatgcttttaaagtagcttattttaaatatgaaaatacacagtggctgaagttttctagtcaaaaagaaatactgagattatatctctatacttccccacaaggaaaaatttattacagtatatatcttttttgattttttataaaactacattgaagtagcttacaaaagaaactgactgcttatcctaaaccatgtttcattttcatacttactttttaggagtcaaatgctttttaactactaaagcttctgtaaggttattatattgtaacttataaatcataagctttcwctctgagagtcttgaatgtctcacggcagcaaataataggagtgaggacattgcttttctgggtccacttcctatatcttctctcaggctttacagaaagagttaagaacattaaaaccaaagtctttaaaagaaactaaagaattaccagaaggagggtggttatgaaagtcacctactcattactgtgttcctattcctgagctcatacagaaaaatgctcttattgacaactcttaccaccagagggcacagaaaactagttcttaaaattggcagagtattcttgatggaggctgaacaagaagcctaaggttatctaaaagcctccctttgcacaaattcatttattctatggacttaagattggaaagccctaaaccaaaccctactatttcctccttcatacttgcttagctttcctatcctagctgaactcaatggtagtcaaagtttgaatgaagcagacagatactgctttacattttctcaggtatttgaagtttattctgattacttaataagatgacctaaaactgcctagtactgaaggtgtgcaggaatgtttgccaaataataaatcagaaccaacttcagagtcaaggatgctcactctggagcactctaaagcaatactattcaaaaagagtcaggaaagactgcaccgactcccagaacatattctctcactccgagtggactgtctatatcctgttgctcttcttcctctggactcattccttaccattttctgtcaaaaccaagatactttaaaaggcctccctaaataccacttgctactgactgtatgaataccttcaaatgacttaatggatagatttgcttacttacaggcatcacaggctctgggtcaatttgtccygcttttcgcttaactccctgaggtggtggtggaggcatagctgactcatctatgtttgttctgctggcctccatcactgactcctggaggcggcttggctcttcaataatgggctcatctgcaattggtcatatgaagagaaaacataggtcatacagttttgaacagtattatagaaccatttattttttaaactagtacactaagacatacataatgctttctttaatgaagaaggccagataaaattttagctccataaagatttactcaatttttagcaatatacgtaaaggggaaaggggatcttaaaaggacaagtcctaccctcagracaatgtaggacaattctttagcagactatctaccctgccagttctgaaccttaaakctctggaaagacaggaggcttcatacttaaataaggcaatcagatccaatgcatttccctgcccaaacatttgagtatatctttgattcctcatttctttcactctgacagtataaaggtaaattttaagccaaatactcatgtgaacttcatcaaggaactattccaacagaacaaaccgataacatcacgctgctgatgctgctgttgctggtcctctctaggaacctctggattttcaaattctttgaggaattcatccaaattatctgcctctcctcctttcctcctttttctaaggtcttctggtacaagcggtgtaagacagcgtgtaaagagctattaaaaaaaaaaaaaagaaaaatttcaattataaaataaatctaattaaaaagtcaccactgatttctatcttccaaaggtatgttctaataaatataagtgtataatttagcatggcttttatcatattcacatttcattccatctgaaaatgattttaaatgagttttcaaattataactgcagacattttagataaactaaaaaaactgtatccattatctcacaaccttaagggcttttctatttatctgcctgcagtttattttcatattcatcaattaaaactcatcctaactggtctcatacaaaatagctttaaattgtgttttcttcccttacatatggtaaccttattagctatcatttaaactattttttttttgagatggagtctcactctgtcgcccaggctggagtgcagtggcacaatctcagctcattgcaacctccgcgcccccggttcaagtgattcttgtgcctcagcctcctgagtagctgggattacaggtgtgtaccaccacacacggctaatttttatattattagtacagacgggattttaccatgttagccaagctggtctcaaactcctgacctcaagtgatccactcgcctcagcctcccaaagtgctgggattacaggcatgagcgactgcttctggtctcatttaaactacttttaatggctacataatatttcattttgaaccattcctctacttttaaaatttcaggcttctctctttttaaactgtaaagaacaatgtaatcaacattttcacagagatagcttttccacactttgaattacttccttaggaaaaatttctaataatgaaattactgagtcaaagaataaatagtttaaggaccttcttaaaaataattatatgatagggcttacaataaagagcacaatgttgttgctctgggatactttagatttagaatatattactatattctaaatacacggtaaaatcaataggttctgtgcatatctcaacttcagacagaatagtagcctaacagcactgcttcattcaccagacacctgtgagtcttattttggaatactgtggaatgtgagcagaaaagagcctataaatggaaaaacaaacaaacaaacaaaaaaacaaaacaacaaaaaacctcctgaagccagaattctttactgtggaaaaaggagaaccactcatgtttcattcagaaaacatttcaagaattcagatgtaaaacacactaagacgatataactaataatttattacagagagaagtattattaaacacgtcagtggctttttaatgtaaggaaaattcattttacatcttttagaaatgcaggtctcacttatccatatatttcagatgaagttatgcccagtacactgtgatttaagggagaaaaaagaaaatgtgccatacagttgcaaaatacttgatcaaaaatgattcaaaggttttagaatcttttttttttagatgctcaaaatgtatatgaagtaaaaattctgcaaactatattaccttcagtagtctgttattccacaaaggctgagcaggtaaagaaaacagtttttctactcctcctgtctctttccacatcatcaatttcttggtgggcggtgccagatccaaagtagtaacaatatctgaataatcactaagttgggctctaattgtcttgctatccaactctttgacactgtcaacaattagcttcctcttcctcttggcttttgtttctttaactggaatgataataaaaaataagatcattttcctgagaggccagcatggaacacacagctacaagatctggactgactatattcccctgcttttctacacacagactctagcagagaagttaaacttcatgaggatagaacgttttaactcatgttttccaaccaccattcagaagagaggctggaacacaacrggcattcaacaaaaatttgctgaatgagtatgtatagacttttagcacaaaataagcaaaatgacgcaagggccaacacatttttgcctatgtagttttatactgtaaacaatgaacatatcacactcttcagagttgggctcattttctcatttgaaacagggcccatcactcaaactcacagattgagttttctttaatgattgtaaatactgttatttttattagaaaagttgtaattcacatatggcagttgtggaagctgcttgatagaatgaaacaagcccaagcccagtcagatagacctgttaactccatcctagacattttaagagtcatttctaatccttacaacttacaagtaggttgtattgcctcacttttttaaaatgcagaaaagcgcatggggttaaataaaatcctcaagatttcaccctgtcagacataccccaaaggtttttggttcaacacttaaaaaaaaaatacaaaaagatgttacacagtgagagtctctctctaatctcaccatctaatacagtatatattttacttaattgcccatttccctcatcaatgggaaacccaaaagggtaatcttgctctacttaaaggtaaaaactatgtctaaattacactgtaatcaatttcaagtattctatcctactgtcctcataaaccatctagaaattgttaggttttatatccccattatctcaaattgtctgtcattcagaagcagccttcctcaaattaaataatgtgtcttgcaatgttacttaaaaaaactacaagaaaaagattattctagaaattttgacatttccagtttttaaatttaaaaaaatttcttttttcagaatgtgtggaaaaaggcaaattctaagagtgcataaaatttttaaaaaaatttcttcttgaagagacaggtcttactatatttactgcccaagctggtctcaaactcctggcctcaagtggttttcctgcttgggcctcccaaagtactggattacaagtgtgagccaccacaccagcctctagttttgttttttttttaaaaagaatacttttagttttttccagctcgtagaccaactgcacagcatttagaattgtactttattgaaaaaagctttccaatgttttccagttgttacacaagtaagctgctaactttctgggaaccccaggagactaagatatgcattatcagaaagcttgttctatacttttgtatttcacagcaacttgcacagacattctacaaaatactcaaaattataattggatagaagaaagactctatgacagatgactgtgacataaaagcaagaagctagttaactaaagcaacagtagcasatcagtagacaggccaaaaaaacagaaaaaagcaagaagcctagttatctaatacaacagtagcagatcagtagacaggcaaaaaccagaaataggaccttatgttgtatgctgtataaatctaaaggttcatatgcttaccagttatatcaataggctccaatgcaaatgcttcttcctcatttggaacaagtgttgtttgatcagtcatggttggcattggttcaacgggatccactgaatcaggactatcaggcccacccactgtaaaaaaaaaaaaaaaaaaaaaaagtcacaaaaagctttggtatataacatatatcccacaaatggggaggagaaaaatctcttcatgaaatggaaagaaatcctgttattactaagaaaaaatatttttctagttaatgggaaagccccgccagacttgaacccaattggccaaaaaaatggggaggggtgcaagattaaacatagaatcagaaccagtaatcataagcactgtacttaaagatatttataattcactaaaaactacaacttttagtttgtcatcttctatggtaagtatctttctggatgccatacaatcatccccagaatcactgaacatgaaatgcttacttgatacattatcatcctcatccatatcgtcatgtgcaggctgctctggcaacatcacccctgcctcagagagggcagggggatcatcaaagataccgccatcattattactaataagtttgtcatctgaaatagggaatgtaagttagttataatttgaaaaagaaatgccaaaccacaacagccaatcaatggaaaaacaaaattatgttaaaagggataaaatttaaacgtatactcaacttttatctgtttctcctccaggtagtttataaaagtataaaacacttggtatataaaaaaaaattctcattcttcatttgtattttctagtttatttatctattttaaaaaatattttacttaacacagctatcaaacacaaacagaacagttttaagtactaataaaatttagcattctgagataagcatatttaggttttgttacaacattttaagaggctagttttaccgaaatgtcctattgaaccaactgatcacaattcactcatatatatctaaaagctgttcaagactcagaattagcaactgtttccaaaactttacaacttaataaagaaattctgtttatgctggaataaccatcattcrcacatacctaatattccaccatcatttccttctccaaaattatcatccttatattgatcttcatattctaaatggttaattttctcattcagattgctggtgctctgttcagactctaataggaggttagaagtagtagtgcttactaacatgtcgtcatcctcaaaagcactgccttctctcattatctcacgatcatccattccaaaatcacctaaacaaattttaatttgtcattagtttagaaagattagaaatagcactgtgataaaagaaaactgctaatgatttattctaattatgtcacatttgcttactgaagtcttacttcaaatgttaaaatatctagtttaaactttaatttaattctttagcatctgaattttttaaaatgactcaattacacacagttacaacactggggcatgactagcaaattcaaacttagatctagatttaaatgatcctaaaatatgtttgataaactctaaatatgtatgtatgttaagttacattaacagtcacagctgaaactttcccgttataatgtgtttcatcaagaaaagaatctggttctgggcacagtggcatgtgcctgtagtcccagctacttgaggctgaagtgggaggatcacttgaatccaagagttcgaggctgtagtgtgctataatccagcctgtgaagagccacggcactccagcmtggataacacagcagagaccccgcctcttaaaaaacaatatgccttgctaactagttttgaatacctgtttttgaatagcaagtataacagggaaactaactgttccaaaatggttggtttaaacgtgatcaccacttcaatgttgggactcctcgcagaaatcaatatataggttctytgattgcatatagggagagaagtaggtaaacaattagctccaagtatgaaaataatgcttaatctactggtagaaagccaaattcttttcttgatgaaaatgcattacatgaaattttaagtctttctgtctatagtctggttttcttttgaaattgctattagcaacacaatataacattataattaatggctaataatttgtttctcttttaccaaaatcattttcttgtaaaatactgatgttcccaacttcttctctcatggttatctcttccactctactctgattcaagctgaactgctgggccacatcgatgtcactttaaaagaaggtcaaatacattttagtttcaagtctatgtataagaaaaacaaatcccaattctctctataagaaaaattaaaaaaatatatatgtataacccaccaagttttagaagccttaatatttaatactttcacacattccagtacaatccaagaatgtaagtgatttggaaattaacttgtatttaaatttgaaaagcatcaataatcagcaaatacaatgtaatatgcagaagtcactaaatcaagtacagaggaaaaaattagaaatcatgggctagataggcatgggttttcattaggaccctgcaacttacaggcggtgtaaactctaaagttccctgacttccctgagactcagtttctttctctaataaagaataatctctcatatagtttaaaaatactaaataaaaaataaaaagtatgtaatgctaaacaccaatctaaacactatcagccatatccagaacattaatacctcaaaacaactgaccaagtctgtacaaaaaatagatggcaaggaaaaaaaaaataggaggcggcaaagctgtaatagattctcaggagacccataagctatatcaaccagatgcagtttgtagactttgtttagcctcaactcgaactgggagaaaaaaaaaaaaaaaaaagccgacatgtcaactggggacatttgaatgctgactggctatcatataggcataccttgttttactgcactttgttttactgagtttcgcagatgctgagtttttaacaaaccaaaggtttgtggtaacagtgccttaagcaagtctgttggtgccattttccaacagcacaagcttacttcatgcctctgtgtcacattttggtaattctcatgctatttaaaaatttttcattattatatttgttatggtgatcttgattagtgatctttaaaatttttttccttattttttagtagacatgggggggcgggatcttactatattgcccaggctagtctcaaacccctggcctcaagtgattctcccacctaagcctcccaaagcactgggatcataggtgtgaaccactatacctggcccagtgatctttgatgctagtattttaattattttggagtgccacaaaggatgcccaaataatatgggcaaacttaatcaagaaatgttctgtgtgttctgactgctcctgccctcccccacccccgcaatgtttctccctcttcttgggcctccctgttccttgagacacaactacattgaaataaggccaattaataaccctacaatggctactatttgttcaagtaaaatgaagagtcacatgtatctcactttaaatcaaaagctagaaatgaataagcttggtgaagaaggcatgcctaagctgatttaggctaaaagctaggcctcttgcaccaaacagccaagttttgaatgcaaaggaaaagttcttgaaggaaatgaaaaatgctactctagtgaacatgaataagaaaacaaaactaccttattgttcatatggagaaagtttcagtggtatagacagaaagtttggaagaaattaacattaaccctgatgcatgactttgaggggttcaagacttcagtggaggcagtaactgcagatatggtggaaaaagcaagagaactagaattagaagtggagcctgaaggtgtaactaaattgatgcaatcttggaataaaactttaacaaatgaggagttacttcttatggatgagcaaagaaagtggtttcttgagatggaatctactcctagtgaagatactgtgaatattgctgaaacaactacaaagggtttagaatattccataaacttggttgataaagcagtggtagggtttcagacgattagctctaattttgaaagaagttctactgcgggtaaaacgctatcgggtagcattgtacgctacaaagaaatctcccatgaaagagtcaattgatgcagcaaacttcactgttgtcttattttaagaaattgccgcagttacctcaaacttcagcaaccaccacctgtatcagtcagcagccatcaacatggaggcaaatcctccaccagcaaaaaaactgcaacttgctgaaggctcagatgatcactagcatttttttgcagtatttttaagataagctcactgcttttttaaacataatgctattactgcacacttaatagactacagtacagtgtgaacataacttttataggcactgtgaaacccaaaaatttgtgtaacttgctttactgtggtggaaccaaacccacagtatctccaaggtatgtctatattaaggaataactgataatttttttaaaggtgtgatgggtcatggaggctttaaaaattctttacctctaagagacaaatatcgaactattacagatgaaataagattattgttacttgatttaaaataatctggtgggggttgtgaggtaagctggggaaacagatgtaataagactggcaatatacttgtaacttaaaaagctgaataactgacatataaggttcataatcctaatttctctagttattagctatgtttaaaaatttccataataaaaaattttcaaataaaatgtcaaaaagagagaaaaaagtaaaatagctagatagagcctagaactaatcaacacacatgcagtaacaaaataggtcataacttactctaattagaagccacagatcatttcaaaaagcagagaaatctgtaatggtgtagagtttcttggttgtcagttatgctaataagtcacttggaaagctacagtcacgttccaacatcatggcatgatagctggatcgtaagggcttgagaaaagggaaacataactacagtactctgctataacagccatctctgtgtaactctctcctctcaaatgtgtgtcatgacaatgaccctttgttgctcttcataaaaagttatctcgtatgttcatttaaaacaaaaataaaaacaaaccagaacagaattatggctctgagtatagcctggccaatccagtagctgactttaatatataactctgaaaaatatcatgaagcaacagtttacattccctgctatcattatataacaagcgtatctgtttcagtttacgtaagctttggcctaagcttgtcaaaactctcacatcttagctgtgttaaagctgacatccacatgattttcttacagaagaacaactttctaggttttatgttccatatttatatactatcctatataaatatatatatgcattttagctgttaatgtaaaacattccaaggaaaactatacatttggaagctaatactactgcttattgcagaccaagtcaacaattttttttaaaagaagacacatactctaagtcaggcagtggctgatcaaagtcatgaaattcttcaggtaaagtaatggcattataagctgcttcccgattttcctcaggcaggtcaaccacacctagaaaagaaatgctaagcttaaatatctagctacccataaattatctagggatcagagggagataagtaggtataacttcctaagtttatattctcatagaaaggttgataacatatgcaaaataccttgaagcacaagtacttctctttttccatgctcataaattgtattccttgctttaggaaaaagtcaaagcactaattatgcattaagctcataaatgtttaaacggacatatcaacaaataactagtaatagtttcctaattaaacatgatatttatgagggggaaaaagaataactgcagaagagtaccttaaatgttaaaaggagctggttattttggagaaagaattacacaattttaattttctttatctttttcctatatttttcaagttgtctataataaatatgccttactttgaaagacaaaaaaaagcatgttatttagataaaatgtgcttattggtcacatgttaatttaagtattgtagtctttttctaaatattttacttttaaataaaatcttagatctcagacattttactgttcacatactgacataaactgaagtactagaatgtatcctcagtagaaactgtttaaggcatttcttaataaaatacacattattttaattagaataaaaatttcatcaatgcttttaaaaattatagaaagatatgtgaatttaaatattaattatctwcttagatggtggtgtggttggcagactccagttttataaccaatcattacctaaaacaaaataacaaaaaactacgaagaatatttacttaggaaaaaaagtttaaaatcctaaaattgtcttttctgagctatattttaaaaaatcatcacataacaattagtttccaacattttaacctcttgatgactcaagtctatgtaagctgctgaaaaaagtattaaaaaggcagctccaaaggaagaacaaatgggggtgagctccattacttcacagggagaacagacgcaagtaggtttcctcccttgaagcactgcctccccaagccttgctctatctttgaagggttcctcgtatttcaattawaacaaagcatgtagatttagaaaatgtgtttagctacagtaacacaaaaaacatgagaaactccaaagtaaacaacaacaacaaaaaccaaacaagtttaacaatacctggccgaaaagccatctttatcttaatgaatgcttcattacagtctgcaagaaggtatttggctttcctgtgatagattcgaactactcccagtaagagatgtcctgatgtccgtaatgccattttcacctatgaataaaacattaatcatattccaaaacctgcatttcgtgccattcatagtcttctttttatctttttcctttaaagttgccttataatcacatacatttctctgtccttacctataaattccctgaatatcgaatatcctgaaaagacagagtgactggaaattcacgaattaacacaaacatgaaggaagctaggaagcaagcactaaaccattccatgaattgtgtcattagcacattaaggagcacgtaacacttttggttgccaatccagggccattgtttcttgctaagtcaattctgattttgtttgagaagcaaatgtcccccacattgacagatgtctcattcctctctgctaatacctgttcttcagtgggcatgaaacacagttcagccaattgagtaataggtgaagtatgccagagagtttctgagaaagatttcccccccaaaagaacaacgcctctcttccttccctctttcctgctttgtgaataggtagggcttgcttggtgcttcatgcagtcaccctgtaaaccacaatgctaaagtatgaggacaggaagctaacacactaaggatggagagttcttggatgacataatcaagctgctggaccaatctcagatctccctgtttccaaaattttagttaattaagcctgttatttcccttattgtttaagctactttttaactaagtttccattaattggaaagcataccgatttaaaaaaaaaagtttggaaagcatactaattgataaagaacaatagtatccatagctttttttcaccagaaaatatactcccaaccttttttcgaggcagatcttgatcaacattaacatgggatacagtatgtggaaaagcagtagttgataaaacaatgagataagactccatctaaacacaagtattcaaaattcctctagccataaggaaaatttactgaaagaacaaggcaatatgaactcaaacatccccagatactttcccaaaccacgccataaagcacaacaagttctaaggtaacaaaaaaagcttttaaacatttgcttaatgtttaaattatgattagtatattgttatttgtaaaagaaaatgttgtattttaaattttatgtgtattttctacagcactctaggaccctacttatgactctcaggttctgtatttatgagcattttatgtgacttacagcataaatgatgatgtatatggccatacatttttagacatccaaaattataccaaatttgtgatcaaattcagaatttatcagacaatgaatccctaattttaaaaaaattcaaagtaaaatgttgctcctaatggaaaaacagaatctatttttatgtggtgttataaaatttaaaattaatattgattgaaccttcacctaagaacaaaggagggaaaaaaaacctctgctattatcttatctagaactagcaactttcaaaaagtggtgtttgggcaaaagctttctttctggattagtctggttcagagtttagcctaggagtaggtaaaatgaggtatggcatatgtttttgagctagtgtgtgttaaagtgtaaacataaatataataaagattccaggctacatattctgggaaagctctgcaaatagggccagtggttcttccagtgggggagtggaagagagaagagaaaaagtatgcaagcttgcagttgcaaatgagccacgtgtgacacttatgctaaacaactaaactgacattttcacaacaagaactgcacaccaaagcaaacattagaaatttcaggcccgttcactctgtggaaatatctttacatcctgttaccaaatagataaaagctcagttagaaatggtttaaaaaatttgaagagaaatcactttctctcataaacacattacaatcttttacattattcctgaactgtctgctttcctttgtggctgttcgtgtatatgttcaattatcagattgtctcatactcgtttgtagtgtccctaaagtggcacaaagtactcggtacatatcatgaaaaaaatctgttatccttttaagccactcaatatttgcttacggatgttaaagattagccacacatatactttccagaaaggagtacgatttctacgttttccattaacttatcccagtaagcatacctataaaaaaaattgaaaataccaatgttctcatacaatttgaccrgcagtgcaaactggtatgatctcaccagaacaaactgttagtgcatatcacaaatctcaaaaacatgtccatcacttggcctatcaattataatcctaaaaataaatcttaagaaacacttaagggcgtgaacaaacatttaacaagaaatgtttactaaatggctagtctttaaaaagaaaataaaacaacctgtaatcctaccatctacagaaatctgtaataatcttttggtatgccttctaggctttcctgtgtacatgtacacaagttttctttgtgtatatacataaaaactaacacaatgattgttagaaggctgcatccaggaaatagagcaaaacttacgaaaaggcacagatttcttataaacacccccttcctcacacacagataatcttccccacaattgacacctcccatcggtgtttcatttattaaaatcgataaacttatattaacacatcattaccaaaagtccgtagtttacatcagggttcacacgtggtgttatacattttattagtttgtacaaatgtaaatggcatttaactattatactatcatgcaaaacagttttactgctctaaaaagaatagtctgtgctctgcctactcaaccctccattctctccccaacctaccatctttttactgtctcatttgtatagttttggcttttccagagtgtcctatagctgcaatcatataatatgtagcctttttagattggcttattatactaagtaatacgcatttacagcacttccacgtcggttttttttttttttgagacagggtctcaaataaaagctcctctgaggaacagatcagtaaacaatatttgggcattgccctgactgatgcttatttgccattaaaaataggcaaatcaaaatatatgcacttagagattttttttaaaatctaatctgtaatcatatcaagtctatctagaggtgataaggacttcayataataaagaaacatttctaaggattttccttgcactccaatgcccctacctaaaaaaactgttttagaagttctatttcacatatcaataaacaatcaacaaagtgaataaaaatgtcaacatcaaacatacctttggtgagatgatactctccacgctgctctctaaattacactcgaacacatgggctttggttagcttcttatcccaatgggccgctagccaaattttggccagaggccctcttttactgagaacaaaatgtgcgtagaacattgttctggctggctatgaaaacagaagaaaacctaagaggggaaaaaaaagttaatgtaaacatcatctgacaatttaaatacttatcaagacataagaaatttaaaatttttcttctctctttaatatgaaaactataaaatgcttgagtaaatgtttaatatgtattttaaaaacaacagatttaaaacaaatctattgttttaattttgagagaggaaaaaaaaatagaccccaaaatactaagagctgaaagtaaaagggacaagagtcaaattgctttcatgccttgtttcatttctatcttgattgacagtagaaatagtctgtttactggccaagagctagagaatatatttttcatggcaagaaaagaaaatctatgaagaaagatactcaggcacagataaaaatctgtggaaatttccatttttagaatgccattaaaaatcgcgattctgggataaattatctgtaaaattgctactacacgaaaagtactgtactaagcatttcatattccaccaagcaaagctaagcagatgggctggccccattttacaggtgggtagtttagaaaaatcgaagttcaaaaaaggtaaagaattatatatatagttttcttaacttaagaacttacaaagcatttatctacttataatttaaatattaaatcagggacaatggtacagcagatttatcttatgactgaaagtcttggaatcacaaatatgttactgacattctactttccttcagcactctaatgtattaagaaaggccgcttgaaaaccccagttcagaagagtacattaactgcaggatatttcctttatatcatttaattcccctctttcacggatttaaaagaaatctacttcagagaatgaatttccagatttaaaacaaatctattgttctgatttttttctaaaatgtccacgactagaaaatgatatatgccttgctttacagaaataaaatcaagtgcatgtacaatacataaaattttaatttgaaatagagtcacagggagtcatagatagcaggtcccatgtacttttttaaacctagtttctcccaatggtgacatcttaaataactacaatatagtatcaaaaccagggcactgacattgattcaatgtgtctacagacttttataccattttgtcacatatgcagatgtgtataatcaccactgcaaaaagatacacagctattccatcactacaaaactcatcacattcatccctctctctctgccatttctaagtcttgacaaacaccacctgttctacatttctaattttgtcactttttttcttttaagagatggggtctcattctgtctcccaggctgaagtgcagtgatgtgatcacagctcactgcagccttgaactcctaggctcaagcaatcctcctccctcagcctctcaagtagttggaactacaggcattgagtcaccacactggacttaattttgttgttttgagaaaattatataaatgaatcatatggtatgtgacttcttgaaatggctttttggatctagcacaatgcttctgtgatccaagttgttacgtttatcaatagtatattcccttttactgttggacagcattccgtgagattcatataccaccatatgcttggacattcacctatagaaggacatttagttgcttccagtttttggctattacaaataaacctgctataaatattcatgtacagacttttggtgaacattaagttttcatttctctaggacaactgtcccgaatgtgactgctgacaagtattgttaagtatatgtttagtttttaaagaatctttaatacaattatcaaaatgagtttttttatatttataggcaagattatcaaatttatatagaaaagcaaataaactaaaaaagctaaaactattttgaaaaatagtgagaaaaattagtctatttgatttcaagacttactatatagctataatactcaagattggtattagcagaggaaacacagtcaatgtaacagaactgagaacccagaaatagcccgcacaactaagttcaactaatttttttttttttttacaaagatgcaaaagctatctaatggagaaatgatacatttttcaacaaatggtgctggagtgattaaatacgatctgaagaccagaaaacatgcacgcacgcacgcacacaccccccacaaaacaaaaaaggcaaggaaaaaaccctccacctaaatctcacacattatataaaaattcaaaatagacaaaacttttaaaaaatggcagaggggaaccttcagtacctaaagcttggtaaaaattttgacacatcaaaaacacaacccatgaaggaaaagttcttgctcaataaactcgcctttatcaaattaaacttttgctctacgaaagaaatgcaaagacaagctatagactggggaaaaaatatttgtaaaccacacatctgactagaactcagatatagaatatgtaaagaattcttaaaacttcaggggtaagaagaaaaaaaaactccaattagaaagtaagcaaaagaggccaggcaggctggctcatgcctgtaaacccagcactttggcaggccaagacaggcagatcacttgaggccaggagttcaagaccagtcaggacaatttggcaaaaccctgtctctactaaaaaacacagaaactgctacgtgtggtggcatgtgcccgtaatcccagctactcaggtggctgaggcaggaggattgcttgaacccaggctgtggaggttgcagcgagctgagatcgcgacactgcattctagcctgggtgacacagcaagactctgtataaaaagaaaaaaaaaagtgagccaaagaagagaaatttcacagaagaggtttactgatgacaaataagaacatgagatgttcaattatcctaggcatttgagaaatacaaattaaggccaaaaacattatcaccacacacttaatataagagctaaattaaaaaatagtggcaacatcaaatgctgaaagacacagagaagctggatctcttacactgcaggtgggactaaaatctcttgaattagcttttctctaaaagatacagagtaattatttttcattttttcacagtctgtaaatcttaaagaattttgcttactgtactttcaattaaaaatgtgccaattctttcccatgatgtagagaaaatatattttagttacgcgtatcacaagcaacagatttcttacatgtcaaaaatattttggagggtggggggctgtggtgcaagactactgcagtcaggtttgctttatctccatttccatcacttaagagtaaagagtcttgaaattctcattctctgaatctaatgttcccaaaggattattaattcatactaaaaaaacttttccwcaaaagaaaaaaaaccaggctttgtcaactgtctaatgttgtaaactcttcctcccaaatttggagttaaggaaggaagaaattggctagcggtggtggctcacgcctgccatcccagccctttgggaggccaaggtaagaggaatgcttgagggcaggagttccagaccaccctggtcaatatagcaagaccccgtgtttattttttaaaaaaaagaaattaaagaatgaagaaatactatattctatactaagaactcagacaaaatattcttaaggtacgagcattaaataacacctcaaaggtgctttatttgccatttgttattttatagattaaaacatgcttcccataccggaagagagcctcctatgtttcaaatcagtgcctacttcaagccttaaaatgttatcacaaaacttaagctgctcctactatgttataaataaaatctagagaactttacagaaatccaacatagcagcatgttctgagaaatataatcgctgttgtacaggacaagccccaagttccactatggaagcaagcaactgtatcctaaacaaaaaactccttaatattaagcttctagaatactatctgtsgcatgacaattactaaatatgtgcttaatgaataagtaagtaagatccaccaagtgatctcataattggcatatgtaaaaaattttagacgttttaaaaattaaaactactggcatttttcaacaggtgtcagtagctcctggccagcacttcagctgctggtcagagcaccgtgcctaaaatatcccagctatgcagaggcagagattcctaaacagaagcctgtttggcataggatggggctaaggaaggcagagtgatgctaaaattaatgtgggaaacaattagcaagaggaaatcactctaataactaaaggaagccaaaggagcagtggtggatcgactcctggtgtatatctgaataaggagaaagtgtaattataattgccttttaagcctatttttttttccttgataataccaaacttcatccaatctttaaaaaagtcgattaatctgtttagattttgaagattctatattctgctaaatcctttatgaaaaccctgtgcagaaaatctgcatttgataccagagcacaacttagcatttcatgatttgagaatcatttttttctaaagcggcaagcagtttttttcaattgaccctaaaaaattaaagtctgatgtgaaacagcagaaagattgctattttagaacatattcaagaatacaaaaaatggcaatttaagactgtttcaaagaatcaaactgaggctcatgcttctaggatagtatcagtcattaccaatatttcactcattagaaagcatggcaggactgcggttgtgtaaattggtactgtcctctctgaaggacaatatgacaacattttataaaaatttcacttacactttctagccagaacttgcactttcagtaatcaatacaaaagatatacttggagatgcatattactaaagacaaaaccaaaaaaccaaacacgaaaaaatcctaagaaaacttactaagcactacgtgttttatgtatccaacctcttcatgactgtcctacagaagtaaattactggcccattttagagatggagaaaacagatgaagataagtaaatgtcactagtatttaatggaagtggaacttgaacttcagaatccacacttctaactacaacactatgctgccttgccagagcagaacagaacattcgaaataacctacttggccatcaataagtgatatgaaaaattaataaacccaaacaacggaatatagtcggtcctccttatccatggcttcttgcatccatggactaaaccaacccaggatcaaaaattgttggagggggggacaaaaaaacacaataataaaaaatactactaataaaaaactataacaactatattaactattaacatagcatttacattgtaatatcataaatggagatgatttgaagtatatgggatgttagtaggttatatgcaaatactacactatttcatataagcgacttgagcattcgtggttttggtyctggaaccaattcccctttaatatcaagggacaattgtacagtacaactttaaacaagaatacaacatttgtkctaacatggaaaatctccaggttaagtgcaaaaaagttggagttttaaaaaagaatgctctttctctatatatttacgagttcttgaataagcttaaaatacctgtggcgtacacatgaaactgstgatactggttacctctggaaagtaggtatgcagggcaagtgaggcctacctttttgtactgtttgatatatatatatacatacacacacacacacacacacacacayatattttaactcgggacataaatgtacaccacctattcaaagtaaaaactactatctcagaagtaaagacataatcctgctattgtcattaactagcagttaagctgcctaacctttatgcagttcagtttccctactatgaaaacctgaaaggttgaaatctggtggtttacctaacctttgaaaacgtcaatacctaactcagatcagaacggagctacctcttcaggggagtagggggaaggatctctgaagatatttctaaccctgcacactagaaatctcctctgttttggttctaacatcctttgaccaagagttttacaatcagacagtcgtaggttcaaattaagctccctctttgctcagttgttttgtaacggtgaataagttacttctcaactataaactgggataatattcatcaaaaaagattatgaaaattaaatacaagggcacacctaacatagtgccactgctgatgcgaaaagtaactctgaagacaatcttgcagcaaaacattaaccagccagtataatcatgcctaacaactctggtttgacatttaaactaaaaacacaaccttatttctcaagaattagaaaatttccattctgtaccaaactgtaaaagcgtttcttagaatttgtatgtttcacaaaataattcaccagaaattcttttgtaattctgacttgtgtataaaaaaaagcactatgtctattagttatgcactgctgtcacctcaactatacaaactgttaacttatttgaagatcagatatggcgtatgtcctcatcaatgcctttccaaaaataattagttgcctccgacattaacaaaggtatttaatgacttcacgatttgagaaaccttctcctgcctgtctccaaaattctatagactcgcacwgcccgctggcatttacacccaatttaagaaaatagaaatttttgcctgaaaaccaattactcttctaaccagtataatgacaacaaagattaactttcccttgcaaaaagaagccgataaacacaagcctttcccagaaactcttcgcacaaacgctgttttttttttcacgctgaagacactaacaaatcagagtgaccaagtaaatcccccagggtttccctcttacaaatttgaagaaggccgctaagtctggcagcacaacccgacagtgtcaccccacactatcaaagcggatcattcgcaaaaagcctttttcagcgacgaaaagggcatcaagcatttatcagaaacacacacctcccccagagcgtctctacaacacccgatatccttaatcactttaaagaatgatcagaaaagttcaggggaaaaccaattatgcaaatatccttggaaaaagaagcaacttccctccgtctctttttccacgcgtctgtgaaatattcagcattcccaacgtaaaaattatctttttaaaacgggagggcagcagcagtcactggccgaggggcacccgccgaccccccgccccaccccgcccagccccaccagctccgcactttcttgagccccgcacccgagttcggcggaaggttgctgctcccggggctggcaccgcgggcgccgggcccgccaccgcctcctcccagacagccatttttacccgcggagtaggcggcccggtccccagagcgggctcgttcaaacctcctcccctccctccgcagcccagggtttccccggcctcctgggggacgtgagatggacctgcagggtaacagcctttgtagatctcagaatggatcagaatcatttgttaccgaacaagcgatgatgcgggcccaagctgcatgggtccgggccgagggcgggcccggggcgaagggggctgggcgggtggcgccgccgcttgggcgccgggagggtggcagcacgcgtgggcgcggcgagggctgccttctccctcgccctcccgcccccaggagtccggctccccgacggcagagcggcggggaaagggtggggggagggagctggaggaaaagaaggggtcggccgagtctcttaccttgctctccgctgggagttgggcgggctgggtggcccggggaggggaaaagggtcgggggagggggtggggaaaggggggagcccttgcgaggtgtagcttccgagcagctcccgccgccgccacagccggcgcctcctttccgattcactcaaacaaacaagatggctgccgttacgccgcggctcttcctgccgcccaaatcctcggttcaaatcggcaggatgtttacggtcaaaatggtacctgtgcgcctgcgcagccagcccaagcccccccctccccccagaaggagcggcgcaggcgcaatgactatttccttttctttggaacccgccctctgtttgtggagtccacaactgagcaagcgcaaaggtgattctcttgcgagggtctttgagagttgcggtgttagccaatagcgtaagatagcgcacgcgcagtgctttccagtgcggtgaatatttgcgtttagctttattcttgtgcttgttttaaagaaaaaagctgtcgtggtgcaattttgtgtgcccccaccaaaaaattcattttatggctctatagggatgaaagtaacataaaaacctcaaactaattccataaaatatagaggttcatttattcagtcaacctatattatagagggtcttctgtttgctacacgttgggctctggatataatgattaatataacagagataataactgccttttggaaattttcagtattgcttgggaggaatcttaaaatttcttatcttaaaagacttacttgtaccacgaagaaatttacttttgtttttattgtagagatctttccagtgatgaaagaaattgaagaggacacacaaaaatggaaatatattccatgttcatggattggaagaatcaatattgttaaaatttccatactacccagagcaatctacagattcaatgtaatccctatcaaaatatcaatgacattcttcacagaaagaggaaaaaaatcctaaaatttatatggaaccacaataagaccaagaatagccaaagraatcctgagcaaaagaataaagctggaggcatcacattacctgacttcaaattatactacaaagtgatagtaaacaaaacagcatggtgctggcataaaaacagacacatagactaacggaacagaatagaggacctataaataaatcaatacatatacagtcaactaatttctgacaaagctgccaagaacatacgttgggaaaaggatggtctcttccaaaaattgttctggaaaaactagataactatatgtagaagaatgaaacagatcactatcttttgccatattaaaaaaatcaaaacaaaatggcttgaagactggaatctaagatttgaaactattgaaactactagaagaaaacgttagggaaatgctccagaaccaaatatttcttagtaagaccttaaaagcataggcaagcaaagcaaaaatggacaaatgggatcatgccaagctaagaaccttctgcaaagcaaacaacaaagtgaagagacaacccatcaagtgggggaaaatacttgcaaactactcatctatcacagaattaatagccataatatataaggagctcaaacaatataataggaaaataatccaattaaaattgagcaaaagacctgaatggttatttctcaaaaaagacatacaaatggccaacagggatatgaaaaaatgcttaacatcactaatcatcagagaaatgcatatcaaaactaggatgaggtataatctcatcccagttgaaatggcttttatccaaaatacaggcaataatgaatgctgataaagatgtggagaaagagaaacactcgtacactgttggtaggaatgtatgttagtacagcactgtgaagaacagtatagagattccttaaaaacctaaatatagagccaccatatgagccagcaatcccactcctgagtaaatacccccccccaaaaaaaaaggaaatcagtacatcagagtggtatctgccctctcacatttattgcagcaagattcacaattgtcaagatatggtatcaacgtaagtgtccatcagtggatagatgggtaaagaaaatgtggtacacatacacaagataatatttttcagccataaaaaagaaaaaaagtcctttcatttgctgcaacatggatagaactggaagacattaagtgaaataaaccaggcacagaaagacattagtgtgaatgttcacactaatatgtgggagcttaaaaaaaaaaaaaagaactcattgagatagaaggtagaatgatggttaccagagcctgggaaaggtagtggggagggggcagatgagtacaaaatctgccccctcagatttggtacaaaaatacaattagaaagaaggaataaagtctagtgtttggtagcacaatagggaaactatagttaataacttactatatatttcaaaataactaaaagagtggaattggaatgtttctaacacaaagaaatgataaatacttgaggtgatggataccccaattaccatggtttgttacacattgtatgcttgtattgaaatgtcagacgtaccttataattgtgtacaactattatgaatccataataataaaaacgtattttaaaaagaaattttgtgtccagtttctgaatgtattttcattttaaagtaaaacacacaggaaaagcagacatttaagtccatatactcctcattgtcagcaaccactacttctggacacttccaggattttaatgacacacagaacacctcacttttacccttcttatgctgatataggagatggataagaaagtggacagtcttgtgctccactcactgtgggtccactgtttttgggttggggcaaggttcgtctcactggctggagtccattagctaagtggccatactggccacagctcaaagctttatcaagtgaatttctgttgtagttagccaaatattttatgttcttttggtttgtttttaatggagactttaagaaattggcattaaggatagttgttaaaatcccaagctggggagtgaaggagtacaagcacttttaacattttaagcatgctttccctcgaccaatatagattagatgctcaaagcacttaaatccttcctgctggtgtggctttaagcaaactcaacagtggcagttgttgcatcatctcaaaaaaaaaaaaaatacaagctcattaacaattaaactccttgcaacagaaattagatttctgccagtgaacaatttaattcttcacttcaaaataatttcggtgtatcagctgctaaatgacctcaaaactaatcatgtgggaaaagtctcctgtcaggttaagaatcacacaaccatgttgcatggtgaagacgttgtcgttcatattaaatttcagtttaaatcaatcccagcttcaaactatggccaggtgaatatgcattgccacttcttgtctctcttatattacattagcttttagagtttttgttcatggaaccatatatttttcatctgtatttgaataaaaacaattctcttttcctcatttctagcagcaagtctgattctaatatttaaatcaacaggcttacctttttcttaagacatttatttttctacttttgggggatggttatgaacatgggctttgggttcaaattcttctcttctacctcccagctgttttaacatgtacaattaatgtctgagctttgatttctttattaataaaatgagggtaatacttgtcatgatgtttgaaataattcaataggatcacatacacaaagtgcttagtatagtgcttggaactgagtaattaataaatggcaggtattactctagttttatctgctttctgaataataagatatcaagcaaggtatgggaaatatttatcagatattcaatttataagaacatcagtaaacaccgggatactctgccccattctgactatatcctgttttatgtttatatttgaaaattattctctctctctctctgtcttctctctcctctctctctctgtcttctctctctcttctctgtctctctccatctttctcttacatgctgttctcctaccccaagttacctcccaaagtactacatttcttttctcttgaactatttattcttactcagagctcccaactctgagcccgccttcccatttgtagattttaatatagtttcttcacatacagtcctaatcccctaaatggctggatttgattctttgaacaagttgtatgttttattctggcttggagtctcctcttatcaacatacccatccacctccttgtattaaaatgtcaagttaaatttcttattgcaggagttagctgcagtatctcttctccctggaaatacttatctgatgaatgtgcatttgtcaaaattagtaattgtgaatttgtttgactaaaaccatgagaggacagatttcagttacagattttgtctttcttcagttgcaaatggtatgctttccttttctgattagtcagttgatgacttgtatgatttggggaaatcactgagaaactgggtgttgccactgtcttgagtatatctataaaaacagctgcgttctgtctatatcacgttattttatgatgacaccccccccacacacacaactgagagttttttttgtgagataaaaactgttatggcaaaaaaagagagaaaatatactgttatagaaaaagatgataaaacttacagtagcagatttgtttaagatttacatatactgttgtagacttgctcctctgaactggcaaacttttaaaacaggattctctataacattaaaataattatagaatcccagcactttgggaggccgaggtgggtggatcacaaggtcaagagagcgagaccatcctggccaacatggtgaaaccccgtctctactaaaaatacaaaaattagctgggagtcgtggcacatgcctgtagtcccagctactagggaggctgaggcaggagaattgcttgaacctgggaggcagaggttgcagtgagctgaggtcatgcactccagcctggcgacagagggagactcgagtctcaaaaaaaaaaagtattattattattatagtaagtaggaggaattaggtgaagagaagaataagacaagaaaatagatatttcaatgttcgggaggatgggtattaaacatgtggtgcaccaggtttttaaaaatttttatttaaataaaaatcaaagtataaaatatgatgatcaaagaaaagttcagaaagagtgaatagcctttttaaggttttgggctaagcaaagaaaaatggactgaaagtgactttaaaaactgtagattctggctttccttctagtgccaacagtatccttacaaggggttttggtcaaaaagggaatgcattttaaggacaaaattcaaggaagaaatgtcataatcagacataaacattatatgagatgtaggggaattaaggacaaaatgtcattatttgaaacaattcctaattcactagatattaattaaattagtgataatagtgtgtgcatgtgtgtgagagagagagaaaaagaaagagagagagaaaccgagagaaagagttttgtaagccttagaaaacataggtttcattcacagtcacatcctcaacttcaagcagaagctgaggtttgagttttaacgtttgagctgaggtttggggtttgaggcaggactccagaaagcggctgcaagcatccctattatggctgggctaagggtctgctcacgcaggtatgtatcagcagtggaaaatcaccaggggccaggagtgggcccagggataatagggggtagtctctgggccagatggaaatgtgagtttatcaaggaccgaagtggagatgaggggaagcctcagaggcagaagctgggtcaaggtcttatgttaatcacttggacttctctcagaaaatttcattatgctaatatttcaatactgggagcatttttaactctagctgggaatttttctcagtgagtattttgatgcatttcctctattttaaagcccatcatatcttccacaattgtgccgcatctttgagaataatgagtgctagctggataggtagtgtgcacctgtagtcccaactactcaggaggatggcttgagcccaggagttcaagtccagcctgggcaacatagcagacccccagctccaaaaacagaaagaaaaaataatagttgctttctaaaagcagaaggttagtgtttattctgaaatcccctcgccattccagaatgcagacatttttgttttaatacaatatacattgttcctttgattctgtgaaattgcttggaaaaattattgggaaaactcaggaaaaatagggttgagatagatttcttaaaaccaatgcaactaacaaaagtgctaacaatcctaataaaaattattaaatatatgttaaattgccaccaatcaaggaaataatatagcaaatatagtacttttaccacaaagaaggtgaaagtgcttggagagaagggtgtaacaaaaggtagaagttactgagttatgagaaaagagtcaaatgcagagattaagtgcttgctgcagtaagcagtccaaagacacaacatggagataaagtgagccaaaagctaacttgaagtgaatgggcactatgtgcagtcctttgttctctgtggctagatgctcagctgtgttaatgtgttttacatgaacttgatgggatcaaaaaattaatgtgctgggaaacacaaagttacaaaggtaacttccatgccgtttggaaatcattcccctatttgttaatgacatatgaggaattagtgttattatagaaactcacactgcaacttagtacctaagactaaatttggggttttattatcagtgagatctgatatatttatggtagggtggtagagagtgatatttctccacttataatgggctatttaccttggggtcttggctaaatcaggttaaaatttagtagtttggggtggggctggggagtctgcttttttaacagcttcaggtaattccaaggctgccactatggggaccacactgaggatttttctttgatttaattttaaatcatgtttaatttatttttattttcttttatttattttatattaatttctttatttttattttgtattgttctctaaccctcgtagatgaagaggatttttgaaaaatagactttggaatcaagtagaacttggtttgcatcatggctcttctatctagtagctgtgtaaccttcagcaagttgcccaacctctctaaggctgagttttcttatttgtaaaatcaggttaattgtatctaccacctcagtgctgtgttgtttatgtcatgcaatgatgtatataaagcatctagcatagtgtttagcatgttatgtgatctcagtaaataatggttaaagagcatcactcttttgcccaggctggagtgcagtagcaatcacagcttactctagcctcgacctcctgtctcgagtgatcctcttgtctcagcctcccaagtagctgggactacaggcatgcaccaccatgcctggctaatttttcgtagagacaaggtctcaccacgttgcccaggctggtctcaaactcctgagctcaagcaaccctcctacctctgcctcccaaggtgctgggattgtaggtgtgagccaccatgcccagctctaatcacacactcatttgcctcaacatatttactatttttttcctcctgaaatgtacttattctctcttttctgaatcgatctttattctatgctgagttctatggcaaaagctgagaattgtttcccaacatctttgttttttttttagttttgcacatggcacccagctaaatgattacattttccagtgtctcttgcatataggtgtggccaagagaatatgttttactcaaattttatgtgggcattactagaatatatctacaaaatggagcaacctttcttctcattctttattttgctgcttggaatatagataaaatggctggtgctccagcagcattttgggcaatgaagacaaaggataagctaaggatagtaaatcagtgatatggacaaatctgagtacataacaactttgtaaagctgtctcataaacccgcaactggctacctccagacttattttatgtaaatgagaaataaagtcctgtcttgattttttttcctgttatatgtagcttactctaatcctcactgatacaagcacttacctggttcaaagaaagatgaaatacagggggcacaaaagttaatgtctgatctctcctggcgagtagttcaaccttctatgtcatatcagttgtctttcacagagtactattttataacttttttgagagaattttccctcctataaacttcacttccaaatgctcttcctcccttattattcttttattttgatcactctggtaagtctcccacaaaatgtgttcttccaagtctaatcacacattccattcctcttaaaaacataaactatggggaagagaatcaggatctgtaaatcaatatttgtatggccagtttatctaatgtcttttattttcagagtcagtaccttcacagttgaaaaactgaatttacgtgtttaatctcaaaacttgcagttccctaacatctggagacttttggcttcctgacagtatgcatttcctctaaaggcagcatttcattttatctgaaatggttagtctttgataaatttaatgaatcctggaaaactctctattatctctctccaagatgctgcccttaaaaccaaaaagcatctctctttgaccatgtcaagtccatacagggcagttgttgaacatctcaataaaatgatcaggatatgtggtgggtggatagaattttctagctagcacctttcctctttgatacactattgagtataggtaactatagcctttttctctgaatgttccagaatcaatcactcctcattggctaagttgcatctgttataaaacttttttcttatgaactgtgcacttcttgtctgaaattaactggattcaatttatgtcagtgttacttcaaccactgatagaattttaagttgcagaatataagtttttgcactggatgaactctttgtcaactagatagtggaaggtcaggttaaaagggatgatatttagcaattattatttattttttatatgaaaatggcatggtggttttgtttttaaagaatggccttatgcttaaaagatacatactaacatagttacaggtaaaatggtacattatctgggatttacttcaaaataatccccaagaagtagaaaaaggagtggatgtagttcaaaacaagattggctgtgggttgatagttgttgaacttgggtgatacatatatggagggtcattttaatcctctttctacctttgtatatatctctctttacctttgtatttccataaaaaaaggatttagaaaagtaagataattagatggagatatgaatactaattttaataatttaataatgggaatcttcaataagcagatttctaatgattcatagaatattttcaatccgcatttggagtttatcagtgcatatttatatgcaaccatccagttctatgagttattcatctgatatattcaccaacaatgtatcaataggaatacgatttctcattcatacaaccagctattagcacacatgagtgagtttatccactaactcactaggggaagacaaatcggggtgaaatctaattgcttttctaagcaatgcatcggttattttctatggtcatctatattttatattgctttcctagtgccataattcatttctttagcatagtgaggccatgagtgtcagtggaaaaagtgggtcatgatatctgttatggctctaaagattcaatgtcagtcttcatctattcatcctgaggtgaactctgacactccctatgcctgtttggtagccatacctcatttattcattcattctacagcatctacttaataattataaaatgcacctatcttcaatattttaaaacctttccctttgctccatatccactgcaccatttatcaactcccccttacagcaaaagttctgaaagaaatgtccatgttttccctctctaattccttccctttcataatctatcttttttattacagtacaaaacacatagtataaagatcaccattttaactgttttaaattgtacaattcaggggcgcttaatacatttacaactttgtgttacagtcaccagcatctagttccagcacatgttcattaccccaaaagggaaccctctacccattaagcagtcccttcccatttcctcctccctgtagcccctggtgactacttttctatggattgatctactgagtatatttcatataaatcgaatcatacagcatgtggttttctgtgtctgaattctttcacttggtgatatggtttggatcagtgtccctgcccaaatcccatgtcaaattgtaatccccagtgttagaggaggggcctgctggggggtgattggattgtgagggtgcatttcccccttgttgttctcatgatagtgagtgaattctcatgatatctggttgtttaaaaatgtgtagcacctcaccctttgctctcttcctcctgctccagccacgtaagatgtgcctgcttccccatcaccttccgccatgactgaaagtttcccaaggcctccccagaagccaagtagccaagtagatgctgccatgcttcctgtacagcctgcagaaccgtgagctaatcttcttttctttattaattacccagtttcagggtatttctttatagcaatgcaagagtggactaatacacttagtgtaatgttttttagattcatccacattgcagcatgtgcagcatgctgctctaaaatatgtaacattttagtttcatccacactgaagcattgttttttcatggctgaataatattccattaaatggatctaccacattttgtttatgcattcattaattcatggacatttgcgttgtttctactttttatctactatgaatagtgttgctaggaacatttgtgtacaaaaatttttgtttgactatctgttttcagttctttttggtatatatctgggagtaaaattgctgtatcatatgccaattttatgtttaacttattgcagaacagccaaactcttttctatagcgctgaaccattttacattccattaataatgtatcagggctccagtttttcacatgctcaccaacttttattactttctgttctttaaaattaaatccatctcaggccaggtgcggtggctcacgcctgtaatcccagcactttgggaggccgaggtgggtagatcacctgaggtctggagtttgagaccagcctggcccacatggtgaaaccctgtctctactaaaaatacaaaaattagccgggcatggtgacacagcctgtagtcccagctatttgggaggctgaggcaggagaatcacttgaacccgggaggcggaggttgcagccagctgagattgagccactgcactccagcctggatgacagagtgagactccatctcaaaaaaaaaaaaaatatatatatatatatatatataatatccatctcagtggatgtaaaatggtatctcattgtggttttagtgtgcattttccaaatgactgaataatgttgagcatcttttcatgtgcttattggccrtttgtatgtcttctatagaaatatgtctatgcaagtcctttgcccatttttaattgggttgttagtcattttattattgagatgtatgagtttttaatatgtttaaaatgttagacccctatcagattccttatcaaatatattatttgcaaatattttctccactattagtcattcttgagaaagaatagcacaaacttaaaaaatttaaatattctgaattcaattgtgtgggttgaaagtatataggttaaaagagtagtttgtctatatgctcactgttagtagaagtaaaaaatatttcaaactcttgagctgtaagcaacagtggagtctacttctggtatccaaacacttccccaaacattaaagcatttagatgtacaatttaattctcaggctgatcttcaaaatcatgtcttggagacacaattaaaatacatgttaattggtttaaggaagtctaccctaatagaagttcaatttctttgagacttttctgatttttggatctttgcaaaataatatatgataattaattaatgtgctagattatttggccttacatgcctgacattccaccatataagttaatttagtttagaatgcagtaagctcctgacatttgtgacaaaaaagaaaccccatgcagaattcctatgaacctcctagaaatgttgtgtcagtcagggtacagtcaggagatagaaactactcagtaacttgaatggaatatttcaaatataaagaattattataatgaactaagaattaattaataataggattaactaagaagcaataaggagaargctacagaatacaatagtagcagatattggaagtaactattacctctgtagctgaggccaagtacccattgagggaaaagacttccaaggaggccaacattgagatctaaactcctatatggagatggtgtggcccagttcatggcagataaatttgctgaggtcccaaaggtcaggggtggctggaaactgcctgttggggttaccagtgaactaactgggaatcaggcctctggaatgctggtgaaactcactggaaagccaccctctaaggagctaacaagactttacaggggtgctggcaaaacttgctggagcatgaatgccactgggtgttccacacactgctagctgtcaagtgctatgggagcaaaaaggcacacaaaaatcaggaaaagtcccttcctcctgcaatgtccctcttctgccctctgttgacaaagcctaacattgcaccctctgacaaaagagaaacctttacgtagtccagctcctgtatcacaaagcaagacaaaggttaatctggaactgggatgctataaattgataagtggcacagatagtgatttgtgattctttgagggaataatccagmaaaaagatggtttatgaggaaagtttcagaagtggttatttacacagggtagcagtgtgattacttagaagacaaaagcttaataatggctaaaccagaaaacctggtttaaattaaacaattaaaattattttaatttcaatgatctaattaaaataaaaaacagataaacagatatgttgaggataatctgaatctatgagccaaaaaatctccaaagcagagaattctcttaaagcagatcccaaagacaaggtcaaaggtaggtcctatactatcaagtgagaattgataccacatgtcctaggtaagttgttctatgaaatgacgaattgcagatataagcaagcaagtgactttatttgcttgttttgttttgggctgctgtggtgaaggagccatcaaatcagagtgggtatatttttgaggatcttgaaaaaataaaaattcctccttttagaagtacatcaatagaagtacataagttaggtgctgcattcactgctattactaaaaaaagaaaagaagagaaaaagaaatacatctgttaggaatgctttttgctgtgtgtaataaatgacctgaataacagagatttcagaaataaagaataaacacttttttttttcttttgagatggagtctcactgtgttgcccaggctagagtgcagtggcatgatcttggctcactgcagcctccaacttatgggttcaagtgattctcctgcctcagccttcccagtagctgggactataggtgcacaccaccacacccggctaattttcgtatttttagtagagacagggttttgccatattggccaggcttatcttgaactcctgacctcagatgatctgctgccttggcctcccaaagtgctgggattacaggtgtgagccactgcgtggactaaaaacagttaataatctcatataacaaatgatccagaggaagcagttccttgatttcttcaaagacccacctcttttgcatctttctgctccagtatccttagactactgtcttgttgcttcatggacagaacttggtgtttctcgttctttcaccatttagacaagaagaaggaagaaaggagttaatatgtgcatctgcatttatgagtatcaagctgtttgacagatattaattgaacaatcatgtgtgtgtgtgtgmgggggggcagtgtgtctggctgtggaatttaataattagtcaaagcatgtacaattttggccctttagcaaatcttacagatccaaacatgagacttcgtcctagaaatttctttttcaccttatacatgcctgattacacagagggtcattccccaacccctgcacactcatagcaattcttctgctctgaatcttcctggtgcattctgggagttatcctacttatttatacttactacataatatattgaggatgacattttatgtaacagatttttttctggtgccataattctctaatataaaatccttaaaaacataatatatattggtaacaaagattttattctctttctctagtctccctctctgctaaatctaataaatgattaaataataggaaactgcagttcttaaagggcagtctctttgttgttaggatctgatttccatttgagcagtggaagatttaatgttgcaattgcaaagagagaatctctcttttattaacttttatacaatttctcattatctaatttataactttgaagggactattcattccttctgtacttttttggaccactttttaaaaacttatgaaataactctttaaataaatagaaaaaacacagggtcaagtgcagtggctcatgcctgtaatcccagcactttgaaaggctgaggctgaggctggtggatcacttaagctcaggagatcgagaccagcctgggcaatgtggcaaaaccccatctccaccaaaaatacaaaatgaacaaacaaatagccggttgtggtggtgcatgcctgtggtcccagctactcaggaggctgaggtggaaggattgcttgagcctgggaggcagaggctatagtgagctgagattgcactcacaatgaaacagtgtgataaaagtacaaaaaattaacccatgtattgtttaataactttgcattgctaactttcaaatatttctgaaagaaaacatatrgggcctattcatcctctgctctctccaacttcatttgctttctacattgtgaaatctcctgtttccagatttagttcttcagagaagcagcctgtccttggcttgtctgtttgatgttatgtaaataatggatagaattttccctgtccatgttttttctgagatggttttctcaaaaaaatttccatttcaaaagcaaactccacaaatacacaaaaaataactaaacatggaaagaggtttagttgccttagattgtgcaaaagactagaagtaaccgccagattctcagcccctgattgtcccaaatcacacgtcatcaactgactaaccagaaaaggaaaacaagccagttgcagatgaaagaagatttgattctaatacagattgtaaacccacaatgactgttttataagcaaagagtcacttttgtgactgaatttgagttttccactgcaatgtttcttgaaaattcctaaaatatcatacattatacattatgccaaacactcaactattgagcggcaagattttattgtttttgggaacactcttaacaggcattggtaggaaatgcaaagaattccagaaacagagctggactggaaagaacacatttatcattagttccataaatctatgtttcttaggattagaagaggaatttcttggactgagtaggttttaaatattctgagccattggcagagggagctaggattgttctgtgatgttgtgaaatacatatttgatcttcattccatttcctggagtacagctcctaaaatccttggaatctcctgagtgataagagttttttttctttgtatgttactgagataacagtggctaagagcacctaggtagtttcaggatgggggtcactggaaagaccaaggcatgattagagagttgggagtttcagcctccccaaccctactccccaatccctgcaagcagcagagaaaggctgaaggttaagttgatcaccaatggctaataatgtaatcaatcatgcctatgtaatgaagcctccataaaaacccaaaagaatagtgttcagaaaggcttctggattgctgaacargtggaggtgtctggagggtgatatgcccacagagggcatggaaatttcactttcctttccacatacattgccctatgaatgtactctgtctggctattcatccatatcttttgtaatatcttacagttcattttgtttgtgggatcatataaacttgggcttgacttattgttgtcattttattttcctgatactttgttttcttgttgtgaaatggggatacaatactttattagttggggctcagttgtaggtgacaggaaacattaggataatcagaaaagaaagaggttaatcatggggaagagctggttttaggctatgtctctgggattgacttccagaatgtcaaccaagaaccagctcaccaaagaaagggctacctctgcaaccttcagaaagctggggagtcaggaagacactatcccaaactcttgattccagcgtcagaccaccttagctatgaactggagtaagaagctgccaccagcattatagactcagagtcacatggtgcatacaaatccattcaagcaaaatgtatgtcactggcatccctactttccttccttttatttactgattaatgcaatacatatttactgaattgtagtaaagaagtaaaatatgtaatacattagaaagagataaacgtaatacattagaaagggataaatgcaatgacgaaaagtagtgtaggaaatgggattacagaaaattgacrgcatcggccttagtctgttcgtgttgctataaaggaatacccgaggcttgataatttatatatttaaaaaagaggtttatttggctcatggttctgcaggttgtacaagaagcatgatgtcagcatttacttttggtgagagtttcagactacttccacttgtggcagaaggagaagtggagctggcatgtacagagatcacatggtgagaaaaaaagtgagagagaaagaaggaggtgtcaggctctttttaacaaccagctgttgtaggaactaatagagcgagaactcactcattacctagaactcactcattacctcaaggatggcaccaagccattcatgatggatccacctccatgactcaaacccctcccactaggccccaccctgaacattggggattatatttcaacatgaggtttagagaggtcaagtatccaaaccatagcagagtcaaagaagacctccttcagaaggtgatatttatgtgaatatctgaagaaggtgagaggacgaaccatgtgagtctttaggggagaagtgtccaggagagggaggagaaaatgcaaaggctctgagacaggagattccctggaaggcaagcaaaagcaaagaggccagcatggaaggagtggagtgagtgagggaatgagcctgctgtcagaaattcacattgatttcaaagtagaaatttcctaagtaactattgcttggttttctatatagacagtgtataagtattttaaactaaaatattcaaagattttgatataatttaaatgaatatatgctcctttaaatgaaaggacagagctaccaatatcactatatttgaagctactgttattttaaataataatccgtgctattaacaatgtcattatctggtttggtttttctttttcctcttttggagacagggtctcactctgttgctcaggttggagtgcagtggcataatcatggctcactacaacctccacctccctcccacctcagcctcccaaatagctgggactacaggcacatgttaccacacccagatagtattttttgtatttttttttatagagacagggttttgtcatattacccagggtggtcttgaactcttgagctcaagcaatccacctgctttagcctcctgaagtgctgggattacaggcatgagtcaccatgcctggccttttttcctttttttataagactttttttaatcataaaaaaaagtctgtctttgatgtgactttttcatgtagaattaatgagttacatgattgacttctaccttttgaaagattcttacaggcttcatttttataatagcacagttataatagctgaaaaaaatctgagtatagcttgtcagattcatcactgaaatatgttttaagggtgcggaataataagtaggctggctttgacacactgctttttttcaatggaaggctaaaagtgagaaaataaactcatttctgtaggagatagagatttacatttgttttcctttgacaaaatactccattctcacacattatacaccattttaagaagattagtatttgtctgagacaaagtgatcttagactttcagtttaaatggtggcatagaagcaagctggtcttactaccccacctccttgccagaaaaccaaaaacaaatatacagcaacaacccagaatcaaatatgaggatgagacagatcctggggacacagagaaatgagagtactttgagcagatggtgggagaatcagactttcacatccatgacaccccatccccccattctgcctggcaactagcacctggaaaatcttctccaactcatggtttctacaagggaaggagtgagattgagatggccaaccagcttttctaccatcttggatatcttgggaggagacctgttattgtcttaacccaagggaagcactgtggctgcctgaagggagaaacttcacagaggacaggtgaagacaaaggagggaagtggaactaccacccccagccctggaaactctgctctgcaacttggcaaaagaagacatcaaatcagaatggttgttcaggagcatcacaccatagaaggtacgtttcatagtttccttgggtgtaaactcctagctagccttctaacagtgccaggtccctttaggacctcacccattcaaaacggacatcactctgatcatttaccaaagccaaggtgaacctggacttaagacacacctagggccaaaaagaaggctgcaacatagtggtaaagattttctaggcaaatatatccactaaaaaaacaaaacaagcctgtcagaggaaatttgaaaaaataattaatccttcaatacaaaggcatagatgtatccaggagggaatggaatgacattttcaaagtgttcaaagaaaagaaaatctgccatccaagaatattgtgcccagccaaattattcttcaactatgaaagagagataaagtctttcccagacaagcaaaagttgagagaattcaccaccagcagatctgtcttacaagaaatgctgaagggagatcttcagtctgaaatagaaaaaaaaataatgcacaaaaagaaaacttttcaaggtgtaaaacccactggtagaattaagtacgtagacaaaccaagaatactctattcctataatggtggtgtgcaatctactcacaactctgataataaagcccaaaagacaaatctgtcaaaaacaataatagctacagcaacttattaaaagatatggaatgtaaaaaatgtaaattgagacaactaaaactcaaaatatgagggtgatagagttaaagtgtagattttttcgtatgtgtgttttttgcctttatttctggtctttattgtgtgatctaaggtaaatgtcatctgtttaaaataacttgtgatatctatggtttttttttgtaagcctcatggtaaccatgcaaaaacctataatagattcaccaaaaataaaaatgaagaaattaaaacatactaccagggaaaatcacttaaacatggaaaaaaaaaaagactgagaaaggaagacagtagtctcaaaacaaccagaaaataggcaataaaatggcagtagtaagtccttacttatcaataacactgactataaatggtctcaattctccaattaaaagtcataaagtgactgaattgataaagaaacaagacccaactatatgctgccttcaagaaactcacttcacctataaaggcacacataagctgaaagtgaaggggtggaaaaaatattccatgcaactggaaaccaataaaaagcaaaagtagctacacttataacagataaactagagtaaaaagctaaggttataaaaaatcacgaagaagttcactatataatgataaaggagtcaatttggcaagaggatataaaaattataaatatctatgcacctaacatcagagcttccaagtatataaagcagatattaatagatctaaaggaagagatacactgctgtataataataataggatatttaacatctcactctcagtaatggacagatcatccagacagaaaatcaacaaagaagcaccatggttaaactatataccagatcaaataggcctgactgaaatttatagaacatttcacccaactgctacagaatacacattcttctcatcagcacatggcacatcctccaggatagaccatatgttaggacacaacgcaagtctgaacaaattaaaaaatatgtaaattgtatcaagtgtttttttctgaccataatgaaataaaactagaaatcagtagcaagagggacctcagaaaatacaaaaacacatgaaaattaaacaacatgctcctgaaaaaccaatgggtcaatgaggaaattaagaaggaaattttttaaatttctcaaagccaataaaaatggaaatacaacatatcaaaatctgtgggatacatcaaaaacagtcctaagggagaagtttatggcgataaatacatttatcaaaaagtaggaagtcttcaaatacacaacctaacagtgcacctgaagaaactggaaaagcaagaagaaaccaagcccaaaatcagtagaaggaaataaataatgaagatcagagccaaaataaataaaattgagacaaaatttacagaagatagatgaaacaagaagttagttttttgaaaaggaaattgactaacctttagctagactgagaaaaaaagaagacctaaataaataaattcagtaatgaaaaaggagacataataaatgagacttcagaaatacaaagaatctgtggacagtattatgaacaactatataccaacaaattggaaaacccagaagaaatggacaaatatctggacatgtacaattgatgaatattgaactacgaagaaatagaaaacctcaacaaaccagtattgagtaatgagatcaaaggcataataaaaaacctctcatcaataaaaatttcaagacctgatggcttcactgctgaattttgccaaacatttaaagataataaactaacaccaattctactcaaactcttcaaaaaaactgaagaggaaggaacacttccagactcattctatgaggccagcattatgctgaaaccaaaaccagacaagtacacaagaagaaaaaaataaaattacaggccaatatcactgatgaacataaatgcacaaaatcctgaacaaaatactagcaaaacaatttcaacaacaatcaaaaagatcattcatcttgatcaagtgggattattctagggatgcaaggatggctcaacatatgcaaactaataagtgtgacacatcacattcacagaatcaagaacaaaaaccgtatggttatttcaatagatgccaaaaaagcatgtgataaaattcagcattctttatgatgaaaatcctcatcagaatgggtataaaaggaacacaccttaaaataataaaggccatatatgacaaacccatggctaacattgtactgaatggagaaacattgaaggcctttcctctaaggaatggaacaaacacaaggatgcccactttcaccactttttttcaacataacactggaagtcttgactggaatgactaggcaaaaaaaaaaaaaaaaaaaaatcagtaaatttgcaggatacaaaattaacatataaaaatcagcagcatttatatatacaaatagtaaacaatctgaaaaagatattaggaaggtaattgcatttacaatagctacaaaaatatcaaatacctaggaatcaatctaaccaaagaagtgcaaggtcaatacaaggaaaactataaaattctgaaaagaaatgaaattaatataataattaatattgttaaattgacaataatacccaaagcaatgcacagattcaatgcaatccatatcaaaataccaatgacattcttcacagagacagaaaaaaaaattctataatgtatctggaaccacaaaagaccctcaatagccaaggtaatactgagcaaaaagaacaaaactgaaggtatcacactacctgatttcaaagcttactataaatttatagtaacaaaaacatcatggtactcacataaaaatagatacatacaacaacggaacagaatagagaatccagatatagatacacacatttacagccaactcatctttgataaaggcaccaagaaataaaatggagaagggacagtcttttcagtaagtagtgctagaaaactggatatccatatgcagaagaattaaactagacctctatctctcactatatgcaaaaattcagtcaatatggattaaacacttaaatctaagacctaaaaccatgaaactactagaaagaaacattgggggaaaatctccaggacactggcctgggcaaaggtttactctgtaagatctcaaaagcacaagtaaccaaagcaaaaatagacaaatgggattacatcaagctacaaggcttctgcacagcagaggaaacaatcaacaaagtgaagagacaacttacagaatgggagaaaatatttgcacactatccatctgagaaggtattaataaccagaacatataagaagctcaaactatttaatagcaaaaaaattaattaaaaatgggcaaaagattggtattaacatttctcaaaagaagacatacaaatggccaacagttacatgaaaaaatattcagcatttgatatggtttggctgtgtccccaccaaaatctcaacttgaattgtatctcccagaagtcctacgtgttgtgggagagacccaaaatacccagggggaggtaattgaatcatgggggctggtctttcccatgctattctcatgatagtgaataagtcttgtgagaactaatgggtttatcaggggtttctacttttgcttcttcatttttctcttgctgccactatgtaaaaagtgccttttgcctcctgccatgattctgaggcctcccagccatgtggaactgtaagtccaattaaacctctttttgtttccagttttgggtatgtctttatcagcagcatgaaaatgaactaatatggtaaattggtaccagtagagtggggcgttgctgaaaagatacccaaaaatgtggaagcgacttaggaactgggtaacaagcagaggttggaacagtttggagggctcagaaaaagacaggaaaatgtgggaaagtttggaacttcctagagacttgctgaatggttttgatgaaaatgctgatagtgacacgaaaaataaggtccaggctgaggttgtctcagatggagatgaggaacttgttgggaactggagccaaggtgactcctgttatgttttagcaaagagactggcagggttttgcccctgccctaaagatttgtggaactttgaacttgagaaagatgatttagggtatctggcggaaaaaatgtctaagcagcaaagcattcaagatgtgacttgggcgctgttaaaagcattcagttttgaaagggaaacagagcatagaagtttggaaaatttccagcctgacaatgtgatagaaaagaaaaacccattttctggagagaaattcaagccagctacagaaatttgcataagtagcaaggagcctaatgctaatccccaagaccatggggaaaacgtctccaggtcatgtcacagaccttcatggcagcctctcccatcacaggcccagaagcctaggagaaagaagtgtttttgtgggctgggcccagggctgccaagctgtgtgcagcctagagacttggtgccctgtgtctccgctgctctagtcatggctgaaagaggccaatgtacagcttgggctgtgggttcagagggtggaagtcccaagccttggcagcctccacatgatgttgagcctgtgggtgcagagaagtcaagaattgaggtttgggaacctctgcctagatttcagaagatgtatggaaatgtctggatgcccaggcaaaagtttgctgcaggggcagggccctcatggagaacctctgctaggtcagtgtggaagggaaatgtggggttggggtcctcacacagagtccctactggggcactgactagtggagctgtgggaagagggccaccatcctccaggtcccagaatggtagatccactgacagcatgcaccatgcacctggaaaagccacagacactcgacgccatcccatgaaagcagccagaagggaggctgtaccctgcaaagccacaggggtggagctgcccaagaccacgggaacccacttcttgcatcagcttgactaggatgtgagacctagtcaaaggagatcattttgcacctttaaaatttgactgcctgctggatttgacttccccgcatgggccctggaaccccttgttttggccaatttctcccatttggtatggctatatttaccaatacctgtacccccattgtatctaggaagtaactagcttgcttttgattttataggctcataggcgaaagagacttgtcttgtctcagatgagactttggactgtggacttttgggttaatgctgaaatgaattaagactttgggggactgttgagaaggcatgattggttttgaaatgtgaggacatgagatttggagaggccaggggtggaatgatatggtttggctgtgtccccaccaaaatctcaacttgaattgtatctcccagaagttccacgcattatgggagggacccagggggaggtaattgaatcatgggggctggtctttcccatgctattctcatgatagtcaataagtctcacgagatctgatggatttatcaggggtttctgcttttctttcttcctcatttttctcttgctgccaccatgtaagaagtgtcttttgcctcctgccatgattctgaggcctccccagtcatgtggaactgtaagtctaattaaacctctttttgtttccagtttcgggtgtgtctttatcagcagtgtgaaaatgaattaatacatcatcactaatcatcagagaaatgcaaatcaaaatcacaatgaaatatcatctcattccagttaaaatggtttgtatcaaaaaggcaataacaaatgctggtaaagatgtggagaaaggggaacccttgtacactcttggtgggaatgtaaactagtacagccaatatagacaacagtatggagcttccgcaaaaactaaaaacagaactaccagcaattctaatactgagtatatatacaagaaaaaggagatcaatatatcaaagagatatctgcattcccacattaattgcagcactattcacaatagccaaaatatggaatcaacctaagtgccaatggatggatgaatgaataaagaaaatgtagaatatacacataattattcagccataacaaacaatgaaatcctgccatttgcaacatggttggaactagaggctattatgttaagtgaaataagccaagcacagaaggacaaatatcacatgttctcactcatatgtgagaactaaaagagtggatctcatgaagatagaagattgttggttaccaaaggccaggaagagtgggagggagaagggtttgaagggaaaaaaagaatataaatggagccattatcacttaattgtacacttaaaaatggtaagaatggtaaattttatatgtatattttatctcaataaaaaccccacaaaattgcaaaaattattactttgtaattataagcatatactagcctagggattagaaggaatacatggtggtgggttgaggagaaaactatgccaaagacgtgtctaagagagcagtcttaaaaaaggatttcttaacctttgcattgtcattaggggtggacaattttctgttgtgggatgctatcttccgcctaggatgtttattagcagtaatcctggtatttacgtgctagatgccagtaacatctcccctcagccctccacccctgccctgccctgatgtaataaccaaaaatgtctctagacattgctaaatatctcctgggggcaaaattattcagggctgagaaccactaagttggagtcagaagagtgaaacaattttaaaaattagctgggtgtggtggtgcatgtctgtagttccagctactcagcattgcttgagcccagtagtttgaggctagacttggcaacagctctgcatgctgtatagaggtagagaccctgaaaaaagaaagaatgaaagaacgaaagaaagcaagaaagaaagaaaaaagaaaaaaattgatctaatcttgaaatgtgaaggagacatctcactgcacagaaacatagagaacaactgtttgtaataattattagtttaatatatattttctttgctagattgtaagatgcacgaggacagagactgtakctcttatttttataatcagatcataatctgaaagttagtcgacaggtgtttattgcattgctatgggtctatcaatgggttatgtgcatttgaggcaggataggtagtcaaggaagtgaccatgttgccaggacgcagtgatcgtggtggccatacaaccaacacaggcctcagcgttcgcagtataattgagctcattcaagcaaagctatctgcggtggggacttttccttctagagagcatgtgcacttggattttaccagtcctcaaattggcgctttgctcatttcaatagtgaaaaatacactcctgggtggagatttcagatgctaatgagacacacgatgcatgaacaagcatgtgcagctactgtgcatgtgcacccagaggaccacccagaacatacctactagtaacgcctcttcccaccttcttatgaattattatgtaaaactcccataaagggagtctccctagtgccagtctttgctggctcatccttatgggcagcccaccctgaatcctctctctctcagggtgtcctgtctattctgtacctacctttcaaaatattctttttcttttgcaataaatcactgtatgctgcaccttctttgccaggtgctcttatttaaattcttttaaactaaggagacaagaactgaggtctcacagaaaccatcaatattttggaaataaaagagaggatttagaactgagtctgttcttaaagtgcttcatggcttattttctaattgcttactatacagtagctcttttctgtgctactagataacaaaacctatagtactaaaaaccatgttctttttcaaagaatgcagaaaagagtatgaagtagaggaagtagaggaggaagtagaattaaagttgattcttgatgaatggactagttttcagtgggtggaagaaaaaaatgaatgagggattccggatgtggtgaacagcataagcagagctattcagttaggaattaaaatcattttggtgttgagagcaagcaactatttttttttctcgagtacttactatgtgtcagttactctgttaactggtatgtatatattatttcatttataggactcagccagtgaaggcatgaaagtgttagaatcattttaatggacttgattcaaatgagggtggggaagtataagtaaaagtcaatccaagatattgtctcagctcctggaacagtggttccattgatggcatggctgcttggaaaagtggtcaaaatttattggtaataggaattttctttttgcattttagagctaaaagggggccttcaatgtcatatttgaatctggcaacaaaacaaccaaaaaagatatcctctatgtaataggaaatatgagatcagaatccctgtgagaggacacaggtaaagatgtaagtttggaaatcatctgtcacgggagtggatgaaatggctaaatgggtaaatcaagagaagagctgcttgccaaaatgagagcctcagtggacactacctattagaacacctgacaacaaagagaagatgaagaagaaatcagagaggaaggaggagaaacaggaaagtacagygttgaaaaatatttagaaaggaaatgatacatgattattaggatttcctaccactctgagatgtaaaaagaggcaaagtcagtttggggaattcttcaagtcaagaagcctggttatcttcattcagagaatggaagagctcagcctcacctttcaattctcattggctctgaaacatcataggggtggggtggataagttactttgtagcttttatttctatttgtggctatgatctgggttgtttacactttgtaaataattttacagaaatataacgatattaatgttttgaaaaaggaattgctataggagagcaagtgtggaaggataagacccacaatggaatccaacgtagattagtaatgaagttctttaaacttgtccagcatgacaatgatctagagcaacagaaatgtcttcttggattcattccatgcgagctaacttggcctccrtgctcctattccattgttcctagaacataacactcattgtccatgcacttgctgtcccaatctactcaggaggcttttcctcccaatatctacaaggcttactaactcactctcttcagatcttgctcaaatgttaccttatcaaatcagcctttcctaaccactctttcgaatacagtaactaccatttttaccctagacactccttactctccttacygtgctcaaggaaattaggatagacttattaccatcagatagactatgcattctcttatttatatgtttattgtctgatctccccttacccctgtgcacataccagcaaattatctctatcatgatggagatcttttattttatacttgctgctacatcccccatgttctagaacagttgcaggtactcaatagatgtggaatgaatggatgaaaaggcactatctttataatacmgtgtagtggttaggacagatgactttgaagcaatgttgcctgggtttcaatcctggcagtaccactgattctagttgtgtggaaggttagttaacttctgtgtacttcatttttattgtctctaaaaaataccataataaaaatattgtaagattgtttaaagaattaaatgaattatctcatgtaaagaactgaggacagtgcctggtacagagttgttgctctgttaatgttagctatcactattttttttttttttttgagacggagtcttgccctgtcacccaggctggagtgcaatagtgcggtctcggctcactgcaacctctgcctcccagattcaagcaattctcctgcttcagcctcctgagtagctgggactacaggtgcgtgccagcacacccagctaatttttgtatttttagtcgagatgaggtttcactatgttggccaggcttgtctctaactcctgacttcctgatctgcccacctcggcctctcaaagtgctggaattacaggtgtcagccactgcgcccagccttagctatcactattaatgtggttctgtaatgtgactgaaaagtgtagtactttcaaatctggacgtataaatacatgctatccagaggtcagaagggaagggattgttggggccagagaggcagtgaggagctatagtagactgaatgcaagaatggtcaccaccaattccttctaccctaaaacatgccatttctctgtcaagaggtagagtctatttctcctcttcctttaaatctggtctggcaaagagtgcaatggaagtgatgttttgagacttccaaggaagatcataacaaacttcagcatcctatggggcctcttagaatgctccaagtaaagccagtcactatctaaaaagtttaactactgtgaaccaccattatgtaaagacactcaaactagctatgtagtaaaaccatatggagagagagtgatgcaggtcagcccctagctgttccagccattccagtcaaagaacaaggaatgtgagcaaaaaagacatcttggacattccagcctcagcagatgccatagggaaaaaaatcaaggccactgtcttatggccccaatttagtcacctcatccctcttcagccatttgtgctaccctagatgagggcccagatattttggagcagacataagccatatccattttcccccaaccaaattttatatacagagaattgtgagcataacagactgatttttaaatgcaattgtattttagggcagtttgttacacagcaacatgtaatgggagctggagacattcagtttgcagaaattaggaaaactttaaaatgctctttatattatgaaagcaataataatgaagatcatgataatgacaacaacactggtaggcatattttattaaaggtctagtatatcttgggtactctaattactaaatttacttcttataagtttgagtagtgtatgtgtgatggttaatactcagtgtcaacttcattggattgaaggatgcaaagtattgatccttggtgtgtctgtgagggtgttgccaaaggagattaacatttgattcggtggctgggaaaggcagacccacccttaatctgggtaggcacaatctaatcagctgccagcatggctaggataacagcaggcagaagaatgtgaaaagactacatctttctcccgtgctggattcttcctgcccttgaacaccagactacaagttcttcagccttgggacttggactgacttccttgctccccagcctattgtgggacgttgtgattatgtgagttaatactacttaataaacaaactccccttcatatatatatatatgtatatcttatcagttctgtccctctagagaaccctgactaatacaatatgttaatgtagttagacaagtttgcacatttgcatatggtgatggaatctatccaaccacaactcattttgcttgtctccccaccactcacatgtgaggggtcttgtgaagcatatcaagaatttgagactttatgggttttttttttgtttctttttttttttgagatggagtttctctcttattgcctacgctggagtgcaatggcacgatctcggcttaccgcagcctccgcctcctgggttcaagcaattctcctgcctcagcctcctgtgtagctgggattacaggcatgtgccacaatgcctggacaattttttttttgtaattttaatagagacagggtttctccatgttggtcaggctggtctcaaactcccgaccttaggtgatctgcccgtcttggcttctcaaagtgctgggattacaggtgtgagccaccgccccggccgagactttatcttaaaggcaatgagcaatcattaaagaaattgtatcaggttgctggtacaatcatatttgaataattataaattattttggctgcagtgggaagaaatgttgttgcagtggtcaagtagtcttcaaatgattcactagggtgaaatgatgggtttggcactagggtggagataactgagataaaaaaagaagagttgactgatttaatggaggtgggtaagtattggaaagattgtacccaggacaaatttgaaggatttggggttattctccaagtttttattcacatattcccagaaaagtctcaggagttattctatctggcctgggtggcctgataaattacatgtaatttaatttcctttaatatcattctaagacggtaaacttaactataatttttttggggagaggataggaaggtactttgatactttctcatttacccaagaacagggctttcacaggcatggagagggtgggagagagggtgtttgttcttagattcatgctcttataggatgcatggtggcaggtgtgataatgatgtcttaccttgcaaaataaaaggatgatgctaatagttcaattcctagataaaatcaggatagcagaggaggaatcttgtggaagcttttggtttatttgggagggaactgtaattatgaaattgggtaagatggtgggaggcttcctccctcacaccatcacgtttgtttctgtccttttggctcactgtacatggccaggaataatagtcttcaagtgtcactttcaagctatgaatttcccagaaaatggaacatggatgatttttcttgcttagttcagggccatgtgtattgatttgtatctgatcttgaatttttctaactcaaggtttctacatactacaaggtttctaactcaagaacatttttctaactcaaggtttttactactaatagcagcagttgacatttatatagtggtttctctgtgtcagacactgttctgaacacttttcatgtgttaatttaatcattatgaaaaccttatgaggtaagtaccatcatcattcccattttacagacaagaaaattgacagcagagaggctttgtaactaacccaagatcactaggaagcagtagagggtgtatttaaatcctggaactctagcttcatagactgtgctttttaaccaatgggcttaagttggtaattcttactgatttggttaatcacttactgattttgataatcacttctatttcttgagtatttactatattaaagacactttgatacgtagtaagtgaaagtagacactggcactatttagtttctagaaagaaataaaatattacctatatattcttatataggtactattttcaatcccatttcatgagggggaaactgagacttaggggggtttaagtatctgctggtaagtggcatagtcactttgaattgaggtctacctcataccacaacccatgggatcaactgtcagctaatacctatctagcttatagaaatttggttttgccggttgtctctactgcatcctgcaggtctttcctaatattacaggatctcttgggaataaaacaactctttgcctgaagtccaatgatcaaataaaaaagccaatatctcttttaactgtgcaattcaagcacatctagtgtaattttacaactcatcccagattgcatttattactctgatggcttgttccaaatacagactatggagcccatgtagcttgtttcatgaaatataggatagagtgaggagtcttttggttttctaatatggaagtagaggatgccagcaataactttccatatttccattaagacatatttattagtgcttacactcatatagttgaatctataattttataggaaattataataatttcttcatcccagaaagtcaaacatttccaagtagggaagaaaattgactttcatgtaattttctcagtttatttatgctgaagaggcttttgccatgtgaagttttctgagtatggcttagaggcaaataaatctttcattttaaatagcataaaacatctgggaattagttcttatctattaaattaatagttcatggaactccagttttgtggagtttgtcctaaattcagggtagagtggaaatcatgttccctgatggaaaaaacttggctgctaggccaagattggttttgacaaaaattgcagttcactcgttgatttakttagatgattctcattctactttaccttaagaagatgtcttcatgggattcagcgaatgtttttaaacagatatatgaggcaaacaagaatgagttatcagctaatatcacagatagttgattccatgaatagactgtcaggctgaagtgacccaagatgatgagatacttttcaagaacagtcttcagatggtaaaaattagacagtattccacataagtactcccattatagtaaaaacatcaccttttagaattcaaaacaagtggtaarcatgaaattccctgttaaatgctttctgttatagctactctgataaacattttctgtggcgaatttatttcactaaagatttgcagacccaaacatgcattttcaacatacaaaattgttcccttttgttttaaacagagcactgttatcaagatggggtatgaccttcacagaatgaaactgattgattccttctctcattataaacttttaatgatgatatggaagacccaaacccatacgcaaacataaacccaatataagcacttatcttagtaaggacattttacggaaaagagratggttagctcatctttgttggaataaaaatttaatatttcttaacagtcactgagtgagagtctgctgggagggtagattactggtttcttagaacaagtccgaaacccattaagcctcacgaaaaattgttatgcattttatcaggaagtctaatttcttcccagaggtaagtcttgaataacattaccaaatagggttttatcccataccaggttttggacaatttttcttttatagagataatagggtcttgtatctataaataagcctgattaaaaattaaataagttatttggttaagtctcattaaagaatgtaaatttagctcccacgctcggatatcaaaggtttgtgtttatgaggaaaataaagagagagaatatgtgtgtgtgtgtctctctgtgtgtgtgtgtgtgtgtgtgtgtgcacacatgtgctctccactccctcaatctgtggacacatgattagaaaaactacccttaagcattttgatcacttatggcaaagcaagtgttacaggagcatgttgcaacaaaaccagaaagaatgcaaactggctagccatatgtagaaagctgaaactggatcccttccttacaccttatacaaaaattaattcaacatagattaaagatttacatgttagacctaaaaccataaaaacactagaggaaaacctaggcaataccartcaggacataggcatgggcaaggactttgtgtctaaaacaccaaaagcaatggcaacaaaagccaaaattgacaaatgggatctaattaaactaaagagcttctgtacagcaaaagaaaataccatcagagtgaacaggcaacctacagaatgggagaaaatttttgcaacctactcatctgacaaagggctaatatccagaatctacaatgaaatccaacaaatttacaagaaaaaaacaaacgaccccatcaaaaagtgggcgaaggatatgaacagacacttctcaaaagaagacatttatgcagccaaaaaacacacgaaaaaatgctcatcatcactggccatcagagaaatgcaaatcaaaaccacaatgagataccatctcacaccagttagaatggtgatcattaaaaagtcaggaaacaacaggtgctggagaggatgtggagaaataggaatacttttacactgttggtgggactgtaaactagttcaaccattgtggaagtcagtgtggcgattcctcagggatctagaactagaaataccatttgacccagccatcccattactgggtatatacccaaaggattataattcatgctgctataaagacacatgcacacgtatgtttatagcggcactattcacaatagcaaagacttggaaccaacctaaatgtccaacaacaatagactggattaagaaaatgtggcacatatacaccatggaatactatgcagccctaaaaaatgatgagttcatgtcctttgtagggacatggatgaaactggaaaccatcattctcagcaaactatcccaaggacaaaaaaccaaacaccgcatgttctcactcataggtgggaattgaacaatgagaacacatggacacaggaaggggaacatcacacatcggggccttttgtggggtggggggaggggggagggatagcattaggagatatacctaatgctaaatgatgagttaatgggtgcagcacaccaacatggcacatgtatacatatgtaacaaacctgcacgttgtgcacatgtaccctaaaacttaaagtataataataataaaagaaaaaaaaagaatgcagctgttaacaaaagtatatttcaagccagtaaggtctagttaaaaaatactgagacctaaaagacctgccacacattaaaattgtgaggaaattgattttgccttagcaaaatgataacacatcaatgtawcctgaacaatagaagaaggtaattcatgaggattatctaaaacaagtgagtttaatgtcgtgaatctgatttgttttggaagcagatcattttatttttaaaaatatatatttaatgctatttaaataattttatatgagacctattttatatgagaccttgatttatgttaatctagagtttgacaaaaatacgattttttaagaatgtacatcccagaggctgacaagggattaccatataaagtcacctagggcagggatcatgcttaatttgtctactcagtaggcaattggaagttttgtgttaggtgaacctgttcggtaaaggtgaaaatgtattctgcagttctttttaatctattaagggctaaccaggatatgatactgtaccaaatcatagtcttttgataatggatgaaagaaagaatgtgcttagagttgtgttttacgatttatattttctgcaactagtatgagttactttttacataaaaaatgtgtaaaaatatcttaaacctcaagtttatatagttttggtttaattcctgagttttctcatacatagagatyaatactcacatggccttcagactccattcccacactcccagtagttatactagaagaattgtggaactgtggaaggaattcagccagaataaggttgggctagattttatgattctgcaacattcattatatttctagccattttgaagcacataattagtgtgctaagtcctagggataacaaggtaaataaaaccatatccctgtccccatctagcttgcatgtaggcaagtacacatctcatctgaaatacatgtagttagtcctgagtgagtgaatgatttgggatcctacagagctgaaatgatttcgtcactcattccacctcctgggaggcttagaacagactttataaaataagcaattgacttctcttaaactgagtcttgaagggttgagttgtgaagtaaagaagggaggagggatcaatccagataaaggtaaaggaacctgtgcaactgtagccgcccaattctgtgctcttgctggaraggtgttgagatcatttggaggagaagaggcactctggctttctgagttttcagcgttttttcgttggttccttctcatctttgtgagtttgtctagctttgatctttgaggctgctggcctttgagatttttgtgggaatctttttgcggatgctgttgttgtttttgttgctttctgtttgttttccttttaacagtcaggcccctcttccgtagggctgctgtggtttgctgggggtccactccaggccctattcacctgggtgcctcccttccctggagatatcagtggaggctgcagaacagcagagatggctgcctgctccttcctctgggagctcccacccagagaagcacctacctgagccagtgggaacgctcccctataaagtgtctggcgacctctactgggggatctcatccagtcaggaggcatgggatccaggatccgtttaaggaagcactctgactgccccttggacaagcgggtgtgctgtgctggtgggaatcccactcttccggactgcccggattcctcagagccagcagggggaaagactgagtcagctgatcctgcggagactatggccactccttctgcaaggggctctgttccaggaagatcagagttctgtccttaaacccttggctggagttgctgaaattccagcagggaggccctgcctggtaaggaggtatgggtctggcctaaagaggcagtctggccacaatttgccatagctgctgtgctgcactgtggggaatttctcctgggtccaaactgcccaatctccctggcactggcaggggaaaatggctgacaggagctgcggtgttggctgccacccctccctccaggagctcagtcgtcttagacggactccagcccagcggctgctgagaatctgcacagctccgtgcttgagacccaaggccctggtggcatgggctcatgaggggtctcctgatccatgggttgcagatctgtggaayaagcatggtttcccaggcggggtagcacaatcagtcgccgcctcccttggctggcggtgggagctccccttgtcctgtgcagctcctaggtgaaccacggctccaccctgcctttcctcactctccatgcgccagccacctagtcagtcccagtgagagaacctagatacgttagttgccagtgcaggtcgctcacagttttcattagtctggatgggagcctctgaccccaactgttcctagtcggccatcttggtcctcttagctgttcttttctaaagacctttcgtcttccatataaatgttagattgagtcagtcaagttcctaaataaatcgagctggacttttgattgggattgaattgaatttgtagtttgagttggagtatattgacattattaatatattctttaatactgtatattgtatagtatatcattttaaagatattatatgtatgtatttaaatataaaatgttctatatgcattccatattctgtgttcttcaatattctgttaattcccaggtacttgatggtttttgttgctgctgctggtatctttttttctattatatttttcagctggtcatttgatattactttgtaaataggaaaacatgtacatctaaacaaaatgtttatctttaaaaccaaaagattctctgctgaagttgctrcactaggagtgttcattgatgcccaataaaaacagatttttactctaataattgttcattttcctgtaacaagcttgggggattcacagcagagagaaaattataaatttgctgtttgtcttactttctgttgtctacagtagaaagtaagaggatagtggggtataaatcttagataagttaaataaacattaactttccaaaactaatagtaatagtaacacctattgtagtggacaccattggttgctcccatgtcatccattcctctcttcatcttttcatacagaatctcaattgttttcctcacacagctgtgtgcttcaggagaacctgacctacctacagatccaggggtgctcctgattggtttaaagacagcccattctctcgccagtgattggttcaggtatggggaacatgatccaattctggccaattaaatkatatttgctgggtggattctggggcaagttttctcattcccaaggaagagacacaaaaagacgtgttttctctatctgcttgcatcagagtgtttctgatttaaargtggatttaatataaagtctttatataaaagaactgaaaatagaacaaagggtatttacattaagttcaaaatgcacaagagattttggcaaaaaaataataacaagttttatagttaaaaagagtaagaataatatcaggttaatcaatttttaaaattggggtaaatttacataccataaaaagccgttttcaatgtttagttcagtgagttttgacaattttatacacacctgtaagtacaccccaaacaaaatataaaatattttttatcacccgaggacgttctttcctgtccctttctagtcaaagatcaccctttcaaaggaaatcactttttgagttttgtcgtcacagattagtttaacctgttcttgaatgtgacataactggaatcatacaatatatattttggtgtgtgcctagcttttttatctccatataatgttcctgagatgcacacaaatacaagtagtctgttattttttttttatcggtgagtagtattccacagtacaaatttaatgaaatttgtttacctgttcttttatggatggacatttgagcagcttttggttttatgtattacgagtaaaactattgaatgtttttgtataaatagttttgtaaatgtatgttttcaattctttttgataaacgtctagagaggagtcatatggttatatggctagtacatgcttaacttaatgtgaaatttcctgaatttctttaaagtggttgtatccttttacactttcactgagagttccagctacaccatatccttaaccatcacttagtgtcatgaatctctttcatgttagctcttctagtcagtgtgaaatgctatctcgctgtgatttcaaatggaatttttaaaaattaccaatgatgttaagcaactttgtcatgaatttactgaggatttgtatatcttctgttgtgtagtatctcttcaagttttcacctatttaaataactggatttgctcgtcttttttttttgtttttgatttgtagaagttcttttttattctgggtgttagtttatagattagatgccaggtatatgtattgtaatatttttcctagtctgtggcttgcataatcatttaaaaatgatgtccttatatgaaggcaactgtttaattttgatgaagcccaatttaattcgttttatggttggtgagttttgtgtcctatctaagaaatgtttgtctaatccaatgtttcaaatatatatatttttttctagaagcttcatatttttggattttatgtttaggtctgtgatcaatctcaaattaatttttatgtgtccagttagataaggattgaagttcattttcttccatatgaatatccggttccagtagtgttttgtaaaaaaaaaaaaactttctttttttcctactgaattgatcagatcccttggccaaaaattaatttactgtattttagtcagttcatttcttgactctttattctgtcccagtgataaaactttcttattaggtgcagacatattaaaatgtgttatgccttcttaatgattaaaatcctttaagccagtgtccttcctagcacacaaaatttttgaacaaagtcaggtttgttctactaagattttgatgtacagtcatgcatcacttaacaacagagatatgatgagacatgtgcccttgggtgattttgccattgtgtttacatcacagagtgcacttacacaaacctagatggtatagcctactacacacctgggctacaaacctttacagcatgttactgtactgaatattgtaggcaattgtaacacaatggtaaatatttgtgcatctaaacatatttaaatatagaaaaggtaaagtaaaagtttaatataaaaataaacatggtggacctatacagggcacttaccataaatagagcggcaggactggaacgygctgtaggtgagtcagtgtgtgagtagtgagtgaatgtgaaagcctaggacattactagacactactgtagactttataaacacttactcttaggctacactaaatgtagaaaaatacatattctttaacatacatattctaaaaatacatatcctaaacatacatatcctaaaaatacatattctagggaaactaaattaactgtagcttactataacttttgtacttcataaacttttaatctttttaaacttttggactcttttaatcttttttaaacttagcttaaaacacaagcatattgtacagctatagaaaatattttctttatatccttattctaaaggcacttttttctatttttaaattttttatttttattttttagctttttaagctttcttgtcaaaaataaagaaacaaacacacgcattagcctaggcctacacagagtcaggatcatcaagatgttgctggatgataggaatttttcagctctattttaatcttatgggaccaccattgcatacgtggtctgccattgacccaaacattgttatacagtgcatgactatacttgtctaatactatcccatctcacaaagcaacctcagtgaattaaaactcatcaytacctcaagtttccaaggccatgtttagcccctgaggactcagttgctcacttttccttgtggcaatttaatgctgcagcagtgtaagaaagatcatcgtagctgcatttccaagcctcatcctccaataaagcttttgtcaataattttatttcttagtgaatgtcaagctcaagagtggaagaaaagagtattacttattggcctcttaaaacstcactcacttgaagggctgctatgtggggttaaaatagatataatagtttggttttctattagacttttgacatgttaactgataagcattctggttggcttgacagttgtattttctttttgcaaattaataaatgactcttttggtccatgagaacagatagcaaaaatgtgcctaattataccatagaatcaatttgtaggtcaaatcaactttcaccagaatagtgtctccctgcaaatttgatacataaagcttattaattgtgagtctttcttctaatgacaacattataaataggctttatctaacatttttagaattctcaccagaatgggaacatttctacttaaatagaaaagctaaaagcaaagatattttgccagttagttattttagttactaagcacttctcaatagatatttatgattgctggactatggcatcaaattttgtctcactaataacttcttgagattacaaagtccaatagcaataatttattatattaattaagagacatcgtagtgatgtgtttaatagtaataggaattagacaatattattaaggatttggtgtatatataaaataatgctattcatgactaaaatttatcttgattcattttctaaaaataactcaatatatttcatgtctctagtacttttattacaattctctatgaatatcctttagcttaggtaggatatttcattaagcatacatcatgctaactcaaggcaggaaataataaaattatgtgaagtgttaacatatctgagagtaaatgataaaaattaatctaaccaagatgccagccataaaataaattacatgggctgaagaaaaatgtgaacatcaaataaaaaagaagaattgcattctaaatctaaggcaaggtagatgagtagagttgagttccttataaagaaaratttgaagtcataactataactagactttacttcaaatgaacaaatctcaattcagattctttcttgccctgagctgcagctaatatgtctccatttcacgacgaatactgtcaggaatgacaatgaccgataagtagaaccagtacaaagttcctcctagaatggaatgtcacccatgtgagggaaaagaagacaaattaagacyaaaattaaaatatatcaccagcagccccaaagcactgactcaaaatcaggtcagtttaaacgtttgttatatgttacttcaatttttaatatttcctggataagtaaccagtaagtgggggattttcccaacagaaaacaaatgtctctctaggaaaactaaggcagtgaattgtcaacaagaggaacagtcaatgaaggcaaaaaaaaaaaaaaccaaaaaaaaaaaaaaaacacaaaacactgrttaattgctgtttcaaatttttccattcaatttgtaaaatttcgwttttttttttttacaaaatttcctcttagttgacacatgcttattatagaaaaaatgttaaaataaaagaaacagaaagcagaaaagtaacctatgcaatgtttagcactcagagaaaagcaatttaaatgttgttccacaccctcctgaacttttattatttttttaacataattgtgttcatattgaatatacattttttgtattctgggtatttactcacttaatgtgacataggtgtgtttgcatattattacatactcttcataaagataattcttaacaacttcacaatattctgacaagtatatgtgttcccagaagtatgaatattttattaatttgacacacattaaattgatacatatagacaattacctttccaagaattgtactgatttacaccattatattatgataccattatattgtgtgagcatgcctatttcaccacattttcaatagcattaatctatttctggactgtttattccaagatttatcttttttcaccagttttgtaaaaactattacacactttccctcacattaacattgaaattgcttctaaatgcaattaataaaaataatattgagattttgattgtgtctctttccgtcaaacctttccatgacttcccttttgtctagttcagtggtgctcagccaggggcgattttatcccccaggagaaattggacaatatctgggggcattttggttgtcccaactggagggtgggactgggggaggcagggttagtggagagtcttgctactggcatctagtgtttaatggccagggatactgctaaacattcttcattgtgctgcataagacagacccccaacaacaaaggatttccccaagttttccaaaatgtcaatagtgtgggtgggggtcaaaaaactctcatctatgggatatattggaaacaactttgcttggcacaccagcctgtctttcatccaactcaatagtcttctaactgaggtacttaaaagccctggtggttggtttctagatcctcaaaatgatttgcaggataatatatccatgttcaaaataatactacaggagaaagaggagaggagggaggacgagaaagaggtatatctttcatctatttcaaatcatgctatgttgcaatgcctctagggtgaaaagaaaaagcaaactaggagcacgactttaaagattaatcaaggtaacgcataaatccatgtggcttcaaactctttataacaaaatgagatacagtgtgcatttaatatttggtaaggatagtcttatcttttcttgtttaaattttttctagatatttccacagacttacaatttccatttgaacctgaaaactggttcagtttaaaaaaattcccgtccaattttttacaatcaatgaacctacattgaaatatcattatatgtacatatacatataaattacatttctataaatttgtcctaaggaatgaagtagaattggatagaaagatatgcaaataacactaacatttacagcatgcttacaatgtgccagcattctaatacatcttaatgtctcagttaatcatcacaactacgctcaagttggtaacactgcgatcttcactttatgaagaaaactgaggctcagaacaggaagttgttccaagtcacagatctatgaagattatagatcaaaccacattctgtgtgatgccaagaccctatatccagctttttctaaatagcagtcttttgggtttcacttatgtgtgtgtgtgtctttgtgtctgtttcaaagtagttgaacaacaatatcagggaaatcatggttttaacgttttaggtatatttcttatatttcccaaaaaaccaatttgaaaatattctacctacgagcctctcagagaggctgtctcttttatatccaggaaccagcactttgcttcaaaggctaacacaacaatggcctgaaggcaggaccagtgttgtgttagtaaagttgtgtagagtaaaacatgtttcaggatctgtcagggacttcagatactttactgatggcattgaattcctgggcttgacctattactgcacctccagcatccaagagcactgtcttacaacttactccttatgactcacctataacaaacatcactgattacatcaaagaaaattattgttaatgccacttagactaattaacacaacctttggtgtagataccaataagaatagtttaagtaccaagtaatctactcttaaaatgttgattcttcttaggatatttcagtgctaattaattgtccagaggatgtggattagggataatgtgcattatgctatcaagggcaagctcacatgcgtgtaggcacacacacacacacacacacacacacatcaccccccaaaatgtttarattgaagcaatttaaagtaaattatcacacagactacagaatagtctcagcaaacagtaactaccagaattatctttccagtgttacttataataataacaaaatttattgaaatgtctaagacgactggcttgatttagaaaatatcttcatacattcttgagtttctcaacttcactatttttgatattttgggctggagaattctttgtcttgggagagattgtcctgtgcattgcaagaagtttagaacccttggtgtctcccaagtacatgccagtagtagctcccttttctcagtagtgccaatacaaaatgtctttagacatcgtcaaatgtcccctgggggcaaaattgcacctagttgagacctataaaatggactacaacccatgtagctattaagagtatgttaaaaaaatatttacttatctacgcaaatgttcatagtatatttaatggaagaatcaggacaaaacaacagataagttagaccttgatttataaaataaaaatggatacatgcattgaaatacaaagacagctagccttttttgtgtgcttactctgtactgcacactaacctttcacatgtgttatctgtttaatttccataataaccctcagaggttggtaattaaccccatttgataaataaggaaacagaatctcagacaggttaagcagctgaatgataattttataatattaaccgcagtcatgtctgggtagtgaaattgggtgtaattatttatctctgtattttccaagatttcaacaataagcatctattatttttaatcagaaaacttaacaaatgacattattgggccyggcacagtggctcacacctgtaatcccaacactttgggaggccgaggtgggaggatcacttgaggtcaggagttca

SEQ ID NO: 2 - DNA, 1896 bp, Homo sapiens
  CDS (1)..(1893)
atgttctacgcacattttgttctcagtaaaagagggcctctggccaaaatttggctagcggcccattgggataagaagctaaccaaagcccatgtgttcgagtgtaatttagagagcagcgtggagagtatcatctcaccaaaggtgaaaatggcattacggacatcaggacatctcttactgggagtagttcgaatctatcacaggaaagccaaataccttcttgcagactgtaatgaagcattcattaagataaagatggcttttcggccaggtgtggttgacctgcctgaggaaaatcgggaagcagcttataatgccattactttacctgaagaatttcatgactttgatcagccactgcctgacttagatgacatcgatgtggcccagcagttcagcttgaatcagagtagagtggaagagataaccatgagagaagaagttgggaacatcagtattttacaagaaaatgattttggtgattttggaatggatgatcgtgagataatgagagaaggcagtgcttttgaggatgacgacatgttagtaagcactactacttctaacctcctattagagtctgaacagagcaccagcaatctgaatgagaaaattaaccatttagaatatgaagatcaatataaggatgataattttggagaaggaaatgatggtggaatattagatgacaaacttattagtaataatgatggcggtatctttgatgatccccctgccctctctgaggcaggggtgatgttgccagagcagcctgcacatgacgatatggatgaggatgataatgtatcaatgggtgggcctgatagtcctgattcagtggatcccgttgaaccaatgccaaccatgactgatcaaacaacacttgttccaaatgaggaagaagcatttgcattggagcctattgatataactgttaaagaaacaaaagccaagaggaagaggaagctaattgttgacagtgtcaaagagttggatagcaagacaattagagcccaacttagtgattattcagatattgttactactttggatctggcaccgcccaccaagaaattgatgatgtggaaagagacaggaggagtagaaaaactgttttctttacctgctcagcctttgtggaataacagactactgaagctctttacacgctgtcttacaccgcttgtaccagaagaccttagaaaaaggaggaaaggaggagaggcagataatttggatgaattcctcaaagaatttgaaaatccagaggttcctagagaggaccagcaacagcagcatcagcagcgtgatgttatcgatgagcccattattgaagagccaagccgcctccaggagtcagtgatggaggccagcagaacaaacatagatgagtcagctatgcctccaccaccacctcagggagttaagcgaaaagctggacaaattgacccagagcctgtgatgcctcctcagcaggtagagcagatggaaataccacctgtagagcttcccccagaagaacctccaaatatctgtcagctaataccagagttagaacttctgccagaaaaagagaaggagaaagagaaggaaaaagaagatgatgaagaggaagaggatgaagatgcatcagggggcgatcaagatcaggaagaaagaagatggaacaaaaggactcagcagatgcttcatggtcttcagcgtgctcttgctaaaactggagctgaatctatcagtttgcttgagttatgtcgaaatacgaacagaaaacaagctgccgcaaagttctacagcttcttggttcttaaaaagcagcaagctattgagctgacacaggaagaaccgtacagtgacatcatcgcaacacctggaccaaggttccatattatataa

SEQ ID NO: 3 - protein, 631 aa, Homo sapiens
MFYAHFVLSKRGPLAKIWLAAHWDKKLTKAHVFECNLESSVESIISPKVKMALRTSGHLLLGVVRIYHRKAKYLLADCNEAFIKIKMAFRPGVVDLPEENREAAYNAITLPEEFHDFDQPLPDLDDIDVAQQFSLNQSRVEEITMREEVGNISILQENDFGDFGMDDREIMREGSAFEDDDMLVSTTTSNLLLESEQSTSNLNEKINHLEYEDQYKDDNFGEGNDGGILDDKLISNNDGGIFDDPPALSEAGVMLPEQPAHDDMDEDDNVSMGGPDSPDSVDPVEPMPTMTDQTTLVPNEEEAFALEPIDITVKETKAKRKRKLIVDSVKELDSKTIRAQLSDYSDIVTTLDLAPPTKKLMMWKETGGVEKLFSLPAQPLWNNRLLKLFTRCLTPLVPEDLRKRRKGGEADNLDEFLKEFENPEVPREDQQQQHQQRDVIDEPIIEEPSRLQESVMEASRTNIDESAMPPPPPQGVKRKAGQIDPEPVMPPQQVEQMEIPPVELPPEEPPNICQLIPELELLPEKEKEKEKEKEDDEEEEDEDASGGDQDQEERRWNKRTQQMLHGLQRALAKTGAESISLLELCRNTNRKQAAAKFYSFLVLKKQQAIELTQEEPYSDIIATPGPRFHII

SEQ ID NO: 4 - DNA, 20 bp, Artificial Sequence
  Primer
atacctgtggcgtacacatg

SEQ ID NO: 5 - DNA, 20 bp, Artificial Sequence
  Primer
aaaaggtaggcctcacttgc

SEQ ID NO: 6 - DNA, 22 bp, Artificial Sequence
  Primer
ctgtggcgtacacatgaaactg

SEQ ID NO: 7 - DNA, 30 bp, Artificial Sequence
  Primer
acgttggatgacaaacggggaaaactcctt

SEQ ID NO: 8 - DNA, 35 bp, Artificial Sequence
  Primer
acgttggatgaatgattcagtttcttcagagtggt

SEQ ID NO: 9 - DNA, 34 bp, Artificial Sequence
  Primer
acgttggatgttcaatatgatgtgcctgtaaacc

SEQ ID NO: 10 - DNA, 35 bp, Artificial Sequence
  Primer
acgttggatgtgacctttctaaaatcaaacattca

SEQ ID NO: 11 - DNA, 30 bp, Artificial Sequence
  Primer
acgttggatgtggattcattccatgcgagc

SEQ ID NO: 12 - DNA, 30 bp, Artificial Sequence
  Primer
acgttggatggcaagtgcatggacaatgag

SEQ ID NO: 13 - DNA, 30 bp, Artificial Sequence
  Primer
acgttggatggagaatgcatagtctatctg

SEQ ID NO: 14 - DNA, 30 bp, Artificial Sequence
  Primer
acgttggatgaccctagacactccttactc

SEQ ID NO: 15 - DNA, 30 bp, Artificial Sequence
  Primer
acgttggatgcactggttaattgctgtttc

SEQ ID NO: 16 - DNA, 30 bp, Artificial Sequence
  Primer
acgttggatgagcatgtgtcaactaagagg

SEQ ID NO: 17 - DNA, 30 bp, Artificial Sequence
  Primer
acgttggatgatagatgagtcagctatgcc

SEQ ID NO: 18 - DNA, 30 bp, Artificial Sequence
  Primer
acgttggatgtacttacaggcatcacaggc

SEQ ID NO: 19 - DNA, 30 bp, Artificial Sequence
  Primer
acgttggatgccagagttagaacttctgcc

SEQ ID NO: 20 - DNA, 30 bp, Artificial Sequence
  Primer
acgttggatggcatcttcatcctcttcctc

SEQ ID NO: 21 - DNA, 30 bp, Artificial Sequence
  Primer
acgttggatgagtgaaatttccatgccctc

SEQ ID NO: 22 - DNA, 30 bp, Artificial Sequence
  Primer
acgttggatggtgttcagaaaggcttctgg

SEQ ID NO: 23 - DNA, 30 bp, Artificial Sequence
  Primer
acgttggatgaataggattaactaagaagc

SEQ ID NO: 24 - DNA, 30 bp, Artificial Sequence
  Primer
acgttggatgctcagctacagaggtaatag

SEQ ID NO: 25 - DNA, 30 bp, Artificial Sequence
  Primer
acgttggatgttgagaaaccttctcctgcc

SEQ ID NO: 26 - DNA, 31 bp, Artificial Sequence
  Primer
acgttggatgcttaaattgggtgtaaatgcc

SEQ ID NO: 27 - DNA, 30 bp, Artificial Sequence
  Primer
acgttggatgttgccatgtgacacacctgc

SEQ ID NO: 28 - DNA, 30 bp, Artificial Sequence
  Primer
acgttggatgaaagcaccagcatctgcttc

SEQ ID NO: 29 - DNA, 30 bp, Artificial Sequence
  Primer
acgttggatgccctgagaagtttaagcttg

SEQ ID NO: 30 - DNA, 30 bp, Artificial Sequence
  Primer
acgttggatggcaaggtaagaggatacaag

SEQ ID NO: 31 - DNA, 30 bp, Artificial Sequence
  Primer
acgttggatgtgtaagatgcacgaggacag

SEQ ID NO: 32 - DNA, 30 bp, Artificial Sequence
  Primer
acgttggatgacacctgtcgactaactttc

SEQ ID NO: 33 - DNA, 30 bp, Artificial Sequence
  Primer
acgttggatgaattccacagccagacacac

SEQ ID NO: 34 - DNA, 30 bp, Artificial Sequence
  Primer
acgttggatgtgagtatcaagctgtttgac

SEQ ID NO: 35 - DNA, 30 bp, Artificial Sequence
  Primer
acgttggatgtttttgcacttaacctggag

SEQ ID NO: 36 - DNA, 29 bp, Artificial Sequence
  Primer
acgttggatgcagtacaactttaaacaag

SEQ ID NO: 37 - DNA, 30 bp, Artificial Sequence
  Primer
acgttggatgaatggagtctgaaggccatg

SEQ ID NO: 38 - DNA, 30 bp, Artificial Sequence
  Primer
acgttggatggttttggtttaattcctgag

SEQ ID NO: 39 - DNA, 30 bp, Artificial Sequence
  Primer
acgttggatgggaaccacaataagaccaag

SEQ ID NO: 40 - DNA, 30 bp, Artificial Sequence
  Primer
acgttggatgtgtgatgcctccagctttat

SEQ ID NO: 41 - DNA, 30 bp, Artificial Sequence
  Primer
acgttggatgaaccatcacccatactgtcc

SEQ ID NO: 42 - DNA, 30 bp, Artificial Sequence
  Primer
acgttggatgtactgagccttgaaggatgc

SEQ ID NO: 43 - DNA, 30 bp, Artificial Sequence
  Primer
acgttggatgatacctgtggcgtacacatg

SEQ ID NO: 44 - DNA, 30 bp, Artificial Sequence
  Primer
acgttggatgaaaaggtaggcctcacttgc

SEQ ID NO: 45 - DNA, 30 bp, Artificial Sequence
  Primer
acgttggatggcagggaaatgcattggatc

SEQ ID NO: 46 - DNA, 30 bp, Artificial Sequence
  Primer
acgttggatgactatctaccctgccagttc

SEQ ID NO: 47 - DNA, 30 bp, Artificial Sequence
  Primer
acgttggatgggaaaggggatcttaaaagg

SEQ ID NO: 48 - DNA, 30 bp, Artificial Sequence
  Primer
acgttggatgaactggcagggtagatagtc

SEQ ID NO: 49 - DNA, 30 bp, Artificial Sequence
  Primer
acgttggatgcaaagtcctctatgtgcaag

SEQ ID NO: 50 - DNA, 30 bp, Artificial Sequence
  Primer
acgttggatgagtgtgtgtagatagcatcc

SEQ ID NO: 51 - DNA, 30 bp, Artificial Sequence
  Primer
acgttggatggcggcgactgattgtgctac

SEQ ID NO: 52 - DNA, 30 bp, Artificial Sequence
  Primer
acgttggatgtctcctgatccatgggttgc

SEQ ID NO: 53 - DNA, 30 bp, Artificial Sequence
  Primer
acgttggatgttgggattacaggtgtgagc

SEQ ID NO: 54 - DNA, 30 bp, Artificial Sequence
  Primer
acgttggatgctgggtagtgaaattgggtg

SEQ ID NO: 55 - DNA, 30 bp, Artificial Sequence
  Primer
acgttggatggcaagctcacatgcgtgtag

SEQ ID NO: 56 - DNA, 31 bp, Artificial Sequence
  Primer
acgttggatggactattctgtagtctgtgtg

SEQ ID NO: 57 - DNA, 30 bp, Artificial Sequence
  Primer
acgttggatggatgagtagagttgagttcc

SEQ ID NO: 58 - DNA, 30 bp, Artificial Sequence
  Primer
acgttggatggctcagggcaagaaagaatc

SEQ ID NO: 59 - DNA, 30 bp, Artificial Sequence
  Primer
acgttggatggtcaagctcaagagtggaag

SEQ ID NO: 60 - DNA, 30 bp, Artificial Sequence
  Primer
acgttggatgtttaaccccacatagcagcc

SEQ ID NO: 61 - DNA, 30 bp, Artificial Sequence
  Primer
acgttggatgtcccatctcacaaagcaacc

SEQ ID NO: 62 - DNA, 30 bp, Artificial Sequence
  Primer
acgttggatgaagtgagcaactgagtcctc

SEQ ID NO: 63 - DNA, 30 bp, Artificial Sequence
  Primer
acgttggatgtgcttgcatcagagtgtttc

SEQ ID NO: 64 - DNA, 30 bp, Artificial Sequence
  Primer
acgttggatgtttgccaaaatctcttgtgc

SEQ ID NO: 65 - DNA, 30 bp, Artificial Sequence
  Primer
acgttggatgcactagaggaaaacctaggc

SEQ ID NO: 66 - DNA, 30 bp, Artificial Sequence
  Primer
acgttggatgtagacacaaagtccttgccc

SEQ ID NO: 67 - DNA, 30 bp, Artificial Sequence
  Primer
acgttggatgaggccaagattggttttgac

SEQ ID NO: 68 - DNA, 30 bp, Artificial Sequence
  Primer
acgttggatgtcgctgaatcccatgaagac

SEQ ID NO: 69 - DNA, 30 bp, Artificial Sequence
  Primer
acgttggatgagagaggaaggaggagaaac

SEQ ID NO: 70 - DNA, 30 bp, Artificial Sequence
  Primer
acgttggatgctcagagtggtaggaaatcc

SEQ ID NO: 71 - DNA, 30 bp, Artificial Sequence
  Primer
acgttggatgcaaatgaagttggagagagc

SEQ ID NO: 72 - DNA, 30 bp, Artificial Sequence
  Primer
acgttggatgactttgcattgctaactttc

SEQ ID NO: 73 - DNA, 31 bp, Artificial Sequence
  Primer
acgttggatggcaagcaactgtatcctaaac

SEQ ID NO: 74 - DNA, 30 bp, Artificial Sequence
  Primer
acgttggatggatcacttggtggatcttac

SEQ ID NO: 75 - DNA, 31 bp, Artificial Sequence
  Primer
acgttggatggtgttactgtagctaaacaca

SEQ ID NO: 76 - DNA, 30 bp, Artificial Sequence
  Primer
acgttggatgtatctttgaagggttcctcg

SEQ ID NO: 77 - DNA, 30 bp, Artificial Sequence
  Primer
acgttggatgaactggagtctgccaaccac

SEQ ID NO: 78 - DNA, 30 bp, Artificial Sequence
  Primer
acgttggatgcagtagaaactgtttaaggc

SEQ ID NO: 79 - DNA, 30 bp, Artificial Sequence
  Primer
acgttggatgggagaaggaaatgatggtgg

SEQ ID NO: 80 - DNA, 30 bp, Artificial Sequence
  Primer
acgttggatgctgtttatgctggaataacc

SEQ ID NO: 81 - DNA, 30 bp, Artificial Sequence
  Primer
acgttggatgtttgctgccgtgagacattc

SEQ ID NO: 82 - DNA, 30 bp, Artificial Sequence
  Primer
acgttggatgctactaaagcttctgtaagg

SEQ ID NO: 83 - DNA, 30 bp, Artificial Sequence
  Primer
acgttggatgttctgtttttttggcctgtc

SEQ ID NO: 84 - DNA, 30 bp, Artificial Sequence
  Primer
acgttggatgctatgacagatgactgtgac

SEQ ID NO: 85 - DNA, 30 bp, Artificial Sequence
  Primer
acgttggatgattgttttttaagaggcggg

SEQ ID NO: 86 - DNA, 30 bp, Artificial Sequence
  Primer
acgttggatggtgctataatccagcctgtg

SEQ ID NO: 87 - DNA, 30 bp, Artificial Sequence
  Primer
acgttggatgcagtttgttctggtgagatc

SEQ ID NO: 88 - DNA, 30 bp, Artificial Sequence
  Primer
acgttggatgcttatcccagtaagcatacc

SEQ ID NO: 89 - DNA, 30 bp, Artificial Sequence
  Primer
acgttggatgagacagttgacaaagcctgg

SEQ ID NO: 90 - DNA, 30 bp, Artificial Sequence
  Primer
acgttggatgtctctgaatctaatgttccc

SEQ ID NO: 91 - DNA, 31 bp, Artificial Sequence
  Primer
acgttggatggttgtactgtacaattgtccc

SEQ ID NO: 92 - DNA, 30 bp, Artificial Sequence
  Primer
acgttggatgaagcgacttgagcattcgtg

SEQ ID NO: 93 - DNA, 30 bp, Artificial Sequence
  Primer
acgttggatgtggtgtacatttatgtcccg

SEQ ID NO: 94 - DNA, 30 bp, Artificial Sequence
  Primer
acgttggatgtgaggcctacctttttgtac

SEQ ID NO: 95 - DNA, 30 bp, Artificial Sequence
  Primer
acgttggatggttgagcatcttttcatgtg

SEQ ID NO: 96 - DNA, 30 bp, Artificial Sequence
  Primer
acgttggatgtgggcaaaggacttgcatag

SEQ ID NO: 97 - DNA, 30 bp, Artificial Sequence
  Primer
acgttggatggtaatcacactgctaccctg

SEQ ID NO: 98 - DNA, 30 bp, Artificial Sequence
  Primer
acgttggatggatttgtgattctttgaggg

SEQ ID NO: 99 - DNA, 31 bp, Artificial Sequence
  Primer
acgttggatggtgtaggaaatgggattacag

SEQ ID NO: 100 - DNA, 30 bp, Artificial Sequence
  Primer
acgttggatgtatcaagcctcgggtattcc

SEQ ID NO: 101 - DNA, 30 bp, Artificial Sequence
  Primer
acgttggatgcaaagtcatctgtcctaacc

SEQ ID NO: 102 - DNA, 30 bp, Artificial Sequence
  Primer
acgttggatgcaggtactcaatagatgtgg

SEQ ID NO: 103 - DNA, 31 bp, Artificial Sequence
  Primer
acgttggatggtattccacataagtactccc

SEQ ID NO: 104 - DNA, 30 bp, Artificial Sequence
  Primer
acgttggatgacagaaagcatttaacaggg

SEQ ID NO: 105 - DNA, 30 bp, Artificial Sequence
  Primer
acgttggatgacctaaaagacctgccacac

SEQ ID NO: 106 - DNA, 30 bp, Artificial Sequence
  Primer
acgttggatgcctcatgaattaccttcttc

SEQ ID NO: 107 - DNA, 30 bp, Artificial Sequence
  Primer
acgttggatgtgcctcttctcctccaaatg

SEQ ID NO: 108 - DNA, 30 bp, Artificial Sequence
  Primer
acgttggatgaggaacctgtgcaactgtag

SEQ ID NO: 109 - DNA, 30 bp, Artificial Sequence
  Primer
acgttggatgaaccaaaagattctctgctg

SEQ ID NO: 110 - DNA, 30 bp, Artificial Sequence
  Primer
acgttggatgatcccccaagcttgttacag

SEQ ID NO: 111 - DNA, 30 bp, Artificial Sequence
  Primer
acgttggatggtgattggttcaggtatggg

SEQ ID NO: 112 - DNA, 30 bp, Artificial Sequence
  Primer
acgttggatgaaacttgccccagaatccac

SEQ ID NO: 113 - DNA, 30 bp, Artificial Sequence
  Primer
acgttggatggacctatacagggcacttac

SEQ ID NO: 114 - DNA, 30 bp, Artificial Sequence
  Primer
acgttggatgctcactactcacacactgac

SEQ ID NO: 115 - DNA, 30 bp, Artificial Sequence
  Primer
acgttggatgtggaatgtcacccatgtgag

SEQ ID NO: 116 - DNA, 30 bp, Artificial Sequence
  Primer
acgttggatgacctgattttgagtcagtgc

SEQ ID NO: 117 - DNA, 30 bp, Artificial Sequence
  Primer
acgttggatggaggaacagtcaatgaaggc

SEQ ID NO: 118 - DNA, 30 bp, Artificial Sequence; identical to SEQ ID NO: 16
  Primer

SEQ ID NO: 119 - DNA, 30 bp, Artificial Sequence
  Primer
acgttggatgttggcccttgcgtcattttg

SEQ ID NO: 120 - DNA, 30 bp, Artificial Sequence
  Primer
acgttggatgccaaccaccattcagaagag

SEQ ID NO: 121 - DNA, 30 bp, Artificial Sequence
  Primer
acgttggatgcctacttctctccctatatg

SEQ ID NO: 122 - DNA, 30 bp, Artificial Sequence
  Primer
acgttggatgaatgttgggactcctcgcag

SEQ ID NO: 123 - DNA, 18 bp, Artificial Sequence
  Primer
aggcacatcatattgaat

SEQ ID NO: 124 - DNA, 18 bp, Artificial Sequence
  Primer
aaaccaaggagttttccc

SEQ ID NO: 125 - DNA, 17 bp, Artificial Sequence
  Primer
gagctaacttggcctcc

SEQ ID NO: 126 - DNA, 21 bp, Artificial Sequence
  Primer
tatcctaatttccttgagcac

SEQ ID NO: 127 - DNA, 22 bp, Artificial Sequence
  Primer
ccattcaatttgtaaaatttcg

SEQ ID NO: 128 - DNA, 17 bp, Artificial Sequence
  Primer
ggagttaagcgaaaagc

SEQ ID NO: 129 - DNA, 17 bp, Artificial Sequence
  Primer
ccagaaaaagagaagga

SEQ ID NO: 130 - DNA, 18 bp, Artificial Sequence
  Primer
ccctccagacacctccac

SEQ ID NO: 131 - DNA, 22 bp, Artificial Sequence
  Primer
aactaagaagcaataaggagaa

SEQ ID NO: 132 - DNA, 21 bp, Artificial Sequence
  Primer
caaaattctatagactcgcac

SEQ ID NO: 133 - DNA, 18 bp, Artificial Sequence
  Primer
ccccctttgccttccacc

SEQ ID NO: 134 - DNA, 20 bp, Artificial Sequence
  Primer
ttcccccaagaaatcaaccc

SEQ ID NO: 135 - DNA, 17 bp, Artificial Sequence
  Primer
cgaggacagagactgta

SEQ ID NO: 136 - DNA, 17 bp, Artificial Sequence
  Primer
agacacactgccccccc

SEQ ID NO: 137 - DNA, 20 bp, Artificial Sequence
  Primer
ctggagattttccatgttag

SEQ ID NO: 138 - DNA, 18 bp, Artificial Sequence
  Primer
gaaggccatgtgagtatt

SEQ ID NO: 139 - DNA, 18 bp, Artificial Sequence
  Primer
gaccaagaatagccaaag

SEQ ID NO: 140 - DNA, 17 bp, Artificial Sequence
  Primer
cttgccactctcctttc

SEQ ID NO: 141 - DNA, 22 bp, Artificial Sequence
  Primer
ctgtggcgtacacatgaaactg

SEQ ID NO: 142 - DNA, 19 bp, Artificial Sequence
  Primer
gcctcctgtctttccagag

SEQ ID NO: 143 - DNA, 17 bp, Artificial Sequence
  Primer
acaagtcctaccctcag

SEQ ID NO: 144 - DNA, 24 bp, Artificial Sequence
  Primer
tttggctgaaagtatgcttctata

SEQ ID NO: 145 - DNA, 19 bp, Artificial Sequence
  Primer
cgcctgggaaaccatgctt

SEQ ID NO: 146 - DNA, 17 bp, Artificial Sequence
  Primer
gtgtgagccactgtgcc

SEQ ID NO: 147 - DNA, 17 bp, Artificial Sequence
  Primer
accccccaaaatgttta

SEQ ID NO: 148 - DNA, 22 bp, Artificial Sequence
  Primer
agttgagttccttataaagaaa

SEQ ID NO: 149 - DNA, 20 bp, Artificial Sequence
  Primer
acttattggcctcttaaaac

SEQ ID NO: 150 - DNA, 23 bp, Artificial Sequence
  Primer
cctcagtgaattaaaactcatca

SEQ ID NO: 151 - DNA, 21 bp, Artificial Sequence
  Primer
tcagagtgtttctgatttaaa

SEQ ID NO: 152 - DNA, 19 bp, Artificial Sequence
  Primer
gaaaacctaggcaatacca

SEQ ID NO: 153 - DNA, 19 bp, Artificial Sequence
  Primer
cagttcactcgttgattta

SEQ ID NO: 154 - DNA, 21 bp, Artificial Sequence
  Primer
aggagaaacaggaaagtacag

SEQ ID NO: 155 - DNA, 17 bp, Artificial Sequence
  Primer
agaggatgaataggccc

SEQ ID NO: 156 - DNA, 22 bp, Artificial Sequence
  Primer
aagcttctagaatactatctgt

SEQ ID NO: 157 - DNA, 24 bp, Artificial Sequence
  Primer
ttttctaaatctacatgctttgtt

SEQ ID NO: 158 - DNA, 17 bp, Artificial Sequence
  Primer
ccacaccaccatctaag

SEQ ID NO: 159 - DNA, 20 bp, Artificial Sequence
  Primer
ggtggaatattaggtatgtg

SEQ ID NO: 160 - DNA, 18 bp, Artificial Sequence
  Primer
cattcaagactctcagag

SEQ ID NO: 161 - DNA, 17 bp, Artificial Sequence
  Primer
ttggcctgtctactgat

SEQ ID NO: 162 - DNA, 18 bp, Artificial Sequence
  Primer
tctctgctgtgttatcca

SEQ ID NO: 163 - DNA, 18 bp, Artificial Sequence
  Primer
cataccagtttgcactgc

SEQ ID NO: 164 - DNA, 22 bp, Artificial Sequence
  Primer
aagcctggttttttttcttttg

SEQ ID NO: 165 - DNA, 18 bp, Artificial Sequence
  Primer
aaggggaattggttccag

SEQ ID NO: 166 - DNA, 22 bp, Artificial Sequence
  Primer
tttatgtcccgagttaaaatat

SEQ ID NO: 167 - DNA, 19 bp, Artificial Sequence
  Primer
tttcatgtgcttattggcc

SEQ ID NO: 168 - DNA, 20 bp, Artificial Sequence
  Primer
tcctcataaaccatcttttt

SEQ ID NO: 169 - DNA, 21 bp, Artificial Sequence
  Primer
atgggattacagaaaattgac

SEQ ID NO: 170 - DNA, 17 bp, Artificial Sequence
  Primer
tgtcctaaccactacac

SEQ ID NO: 171 - DNA, 22 bp, Artificial Sequence
  Primer
tagaattcaaaacaagtggtaa

SEQ ID NO: 172 - DNA, 23 bp, Artificial Sequence
  Primer
caaaatgataacacatcaatgta

SEQ ID NO: 173 - DNA, 20 bp, Artificial Sequence
  Primer
tccaaatgatctcaacacct

SEQ ID NO: 174 - DNA, 17 bp, Artificial Sequence
  Primer
tctctgctgaagttgct

SEQ ID NO: 175 - DNA, 23 bp, Artificial Sequence
  Primer
gatccaattctggccaattaaat

SEQ ID NO: 176 - DNA, 17 bp, Artificial Sequence
  Primer
gcggcaggactggaacg

SEQ ID NO: 177 - DNA, 24 bp, Artificial Sequence
  Primer
agggaaaagaagacaaattaagac

SEQ ID NO: 178 - DNA, 22 bp, Artificial Sequence
  Primer
aaaaaaaaaacacaaaacactg

SEQ ID NO: 179 - DNA, 19 bp, Artificial Sequence
  Primer
caaatttttgttgaatgcc

SEQ ID NO: 180 - DNA, 19 bp, Artificial Sequence
  Primer
ctctccctatatgcaatca

SEQ ID NO: 181 - DNA, 18 bp, Artificial Sequence
  Primer
cttggggtgctgttttct

SEQ ID NO: 182 - DNA, 18 bp, Artificial Sequence
  Primer
attgccacagggagtgat

SEQ ID NO: 183 - DNA, 20 bp, Artificial Sequence
  Primer
ctctccctccagaaaaaata

SEQ ID NO: 184 - DNA, 21 bp, Artificial Sequence
  Primer
ctcagcagcattaagtacagt

SEQ ID NO: 185 - DNA, 19 bp, Artificial Sequence
  Primer
gagttacagcgaagcataa

SEQ ID NO: 186 - DNA, 19 bp, Artificial Sequence
  Primer
tccttgtggggaagtatag

SEQ ID NO: 187 - DNA, 21 bp, Artificial Sequence
  Primer
tggagcactctaaagcaatac

SEQ ID NO: 188 - DNA, 18 bp, Artificial Sequence
  Primer
atcccctttcccctttac

SEQ ID NO: 189 - DNA, 20 bp, Artificial Sequence
  Primer
aagacaggaggcttcatact

SEQ ID NO: 190 - DNA, 22 bp, Artificial Sequence
  Primer
cctttggaagatagaaatcagt

SEQ ID NO: 191 - DNA, 20 bp, Artificial Sequence
  Primer
aaagaaaatgtgccatacag

SEQ ID NO: 192 - DNA, 19 bp, Artificial Sequence
  Primer
tgcgtcattttgcttattt

SEQ ID NO: 193 - DNA, 20 bp, Artificial Sequence
  Primer
aaaaaagcaagaagcctagt

SEQ ID NO: 194 - DNA, 18 bp, Artificial Sequence
  Primer
tttctcctccccatttgt

SEQ ID NO: 195 - DNA, 19 bp, Artificial Sequence
  Primer
tacaatcatccccagaatc

SEQ ID NO: 196 - DNA, 20 bp, Artificial Sequence
  Primer
ctggaggagaaacagataaa

SEQ ID NO: 197 - DNA, 19 bp, Artificial Sequence
  Primer
ccgaaatgtcctattgaac

SEQ ID NO: 198 - DNA, 18 bp, Artificial Sequence
  Primer
tgccccagtgttgtaact

SEQ ID NO: 199 - DNA, 18 bp, Artificial Sequence
  Primer
actcctcgcagaaatcaa

SEQ ID NO: 200 - DNA, 21 bp, Artificial Sequence
  Primer
cttggattgtactggaatgtg

SEQ ID NO: 201 - DNA, 20 bp, Artificial Sequence
  Primer
acaagcgtatctgtttcagt

SEQ ID NO: 202 - DNA, 22 bp, Artificial Sequence
  Primer
tacctacttatctccctctgat

SEQ ID NO: 203 - DNA, 18 bp, Artificial Sequence
  Primer
tgaagggttcctcgtatt

SEQ ID NO: 204 - DNA, 20 bp, Artificial Sequence
  Primer
atttccagtcactctgtctt

SEQ ID NO: 205 - DNA, 20 bp, Artificial Sequence
  Primer
ctgatgcttatttgccatta

SEQ ID NO: 206 - DNA, 20 bp, Artificial Sequence
  Primer
ttcccctcttaggttttctt

SEQ ID NO: 207 - DNA, 21 bp, Artificial Sequence
  Primer
ctttctatcgctttgaataca

SEQ ID NO: 208 - DNA, 19 bp, Artificial Sequence
  Primer
acacagaaccctttgagaa

SEQ ID NO: 209 - DNA, 30 bp, Artificial Sequence
  Primer
acgttggatgacctcttcctcttcatcatc

SEQ ID NO: 210 - DNA, 30 bp, Artificial Sequence
  Primer
acgttggatgaccagagttagaacttctgc

SEQ ID NO: 211 - DNA, 30 bp, Artificial Sequence; identical to SEQ ID NO: 18
  Primer

SEQ ID NO: 212 - DNA, 30 bp, Artificial Sequence
  Primer
acgttggatgagatgagtcagctatgcctc

SEQ ID NO: 213 - DNA, 30 bp, Artificial Sequence; identical to SEQ ID NO: 212
  Primer

SEQ ID NO: 214 - DNA, 30 bp, Artificial Sequence; identical to SEQ ID NO: 18
  Primer

SEQ ID NO: 215 - DNA, 30 bp, Artificial Sequence
  Primer
acgttggatgatcaccacttcaatgttggg

SEQ ID NO: 216 - DNA, 30 bp, Artificial Sequence; identical to SEQ ID NO: 121
  Primer

SEQ ID NO: 217 - DNA, 30 bp, Artificial Sequence
  Primer
acgttggatgtgttctcagtaaaagagggc

SEQ ID NO: 218 - DNA, 30 bp, Artificial Sequence
  Primer
acgttggatgacacatgggctttggttagc

SEQ ID NO: 219 - DNA, 30 bp, Artificial Sequence
  Primer
acgttggatggaagtcttacttcaaatgtt

SEQ ID NO: 220 - DNA, 31 bp, Artificial Sequence
  Primer
acgttggatggagtcattttaaaaaattcag

SEQ ID NO: 221 - DNA, 30 bp, Artificial Sequence
  Primer
acgttggatgattggagtgcaaggaaaatc

SEQ ID NO: 222 - DNA, 31 bp, Artificial Sequence
  Primer
acgttggatgcatatcaagtctatctagagg

SEQ ID NO: 223 - DNA, 23 bp, Artificial Sequence
  Primer
cttctgccagaaaaagagaagga

SEQ ID NO: 224 - DNA, 21 bp, Artificial Sequence
  Primer
ctcagggagttaagcgaaaag

SEQ ID NO: 225 - DNA, 23 bp, Artificial Sequence
  Primer
acaggctctgggtcaatttgtcc

SEQ ID NO: 226 - DNA, 20 bp, Artificial Sequence
  Primer
tctctccctatatgcaatca

SEQ ID NO: 227 - DNA, 21 bp, Artificial Sequence
  Primer
gctttggttagcttcttatcc

SEQ ID NO: 228 - DNA, 19 bp, Artificial Sequence
  Primer
attcagatgctaaagaatt

SEQ ID NO: 229 - DNA, 20 bp, Artificial Sequence
  Primer
tagaggtgataaggacttca

SEQ ID NO: 230 - DNA, 20 bp, Artificial Sequence
  Primer
caatgccaaccatgactgat

SEQ ID NO: 231 - DNA, 21 bp, Artificial Sequence
  Primer
cggtgtaagacagcgtgtaaa

SEQ ID NO: 232 - RNA, 21 nt, Artificial Sequence
  Synthetic oligonucleotide
aagcccauguguucgagugua

SEQ ID NO: 233 - RNA, 21 nt, Artificial Sequence
  Synthetic oligonucleotide
aagaguuggauagcaagacaa

SEQ ID NO: 234 - RNA, 21 nt, Artificial Sequence
  Synthetic oligonucleotide
aagacagauacgaugaugaga

SEQ ID NO: 235 - DNA, 23 bp, Artificial Sequence
  Illustrative polynucleotide sequence
aannnnnnnnnnnnnnnnnnntt

(19) United States
(12) Patent Application Publication
(10) Pub. No.: US 2005/0064442 A1
(43) Pub. Date: Mar. 24, 2005

(54) Methods for identifying risk of breast cancer and treatments thereof

(76) Inventors: Richard B. Roth, La Jolla, CA (US); Josephine M. Atienza, La Jolla, CA (US); Mikhail F. Denissenko, Poway, CA (US); Andreas Braun, San Diego, CA (US); Stefan M. Kammerer, San Diego, CA (US); Matthew R. Nelson, San Marcos, CA (US); Rikard H. Reneland, San Diego, CA (US)

Correspondence Address: Andreas Bruan, 3935 Lago Di Grata Circle, San Diego, CA, 92130, US

(21) Appl. No.: 10/723,518

(22) Filed: Nov. 25, 2003

Related U.S. Application Data
(60) Provisional application No. 60/429,136, filed on Nov. 25, 2002.
(60) Provisional application No. 60/490,234, filed on Jul. 24, 2003.
(60) Provisional application No. 60/504,258, filed on Sep. 18, 2003.

(51) Int. Cl. C12Q 1/68; G01N 33/574

(52) U.S. Cl. 435/6; 435/7.23

(57) ABSTRACT

Provided herein are methods for identifying a subject at risk of breast cancer, reagents and kits for carrying out the methods, methods for identifying candidate therapeutics for treating breast cancer, and therapeutic methods for treating breast cancer in a subject. These embodiments are based upon an analysis of polymorphic variations in nucleotide sequences within the human genome.

### RELATED PATENT APPLICATIONS

This patent application claims the benefit of provisional patent application No. 60/429,136 filed Nov. 25, 2002 and provisional patent application No. 60/490,234 filed Jul. 24, 2003, having attorney docket number 524593004100 and 524593004101, respectively. This patent application also claims the benefit of provisional patent application No. 60/504,258 filed Sep. 18, 2003 and having attorney docket no. 524593006000. Each of these provisional patent applications names Richard B. Roth et al. as inventors and is hereby incorporated herein by reference in its entirety, including all drawings and cited publications and documents.

### FIELD OF THE INVENTION

The invention relates to genetic methods for identifying subjects at risk of breast cancer and treatments that specifically target the disease.

### BACKGROUND

Breast cancer is the third most common cancer, and the most common cancer in women, as well as a cause of disability, psychological trauma, and economic loss. Breast cancer is the second most common cause of cancer death in women in the United States, in particular for women between the ages of 15 and 54, and the leading cause of cancer-related death (Forbes, Seminars in Oncology, vol.24(1), Suppl 1, 1997: pp.S1-20-S1-35). Indirect effects of the disease also contribute to the mortality from breast cancer including consequences of advanced disease, such as metastases to the bone or brain. Complications arising from bone marrow suppression, radiation fibrosis and neutropenic sepsis, collateral effects from therapeutic interventions, such as surgery, radiation, chemotherapy, or bone marrow transplantation-also contribute to the morbidity and mortality from this disease.

While the pathogenesis of breast cancer is unclear, transformation of normal breast epithelium to a malignant phenotype may be the result of genetic factors, especially in women under thirty (Miki, et al., Science, 266: 66-71 (1994)). However, it is likely that other, non-genetic factors also have a significant effect on the etiology of the disease. Regardless of its origin, breast cancer morbidity increases significantly if it is not detected early in its progression. Thus, considerable efforts have focused on the elucidation of early cellular events surrounding transformation in breast tissue. Such efforts have led to the identification of several potential breast cancer markers. For example, alleles of the BRCA1 and BRCA2 genes have been linked to hereditary and early-onset breast cancer (Wooster, et al., Science, 265: 2088-2090 (1994)). However, BRCA1 is limited as a cancer marker because BRCA1 mutations fail to account for the majority of breast cancers (Ford, et al., British J. Cancer, 72: 805-812 (1995)). Similarly, the BRCA2 gene, which has been linked to forms of hereditary breast cancer, accounts for only a small portion of total breast cancer cases.

### SUMMARY

It has been discovered that polymorphic variations of RAD21 loci in human genomic DNA are associated with occurrence of breast cancer. Thus, featured herein are methods for identifying a subject at risk of breast cancer and/or determining risk of breast cancer in a subject, which comprise detecting the presence or absence of one or more polymorphic variations associated with breast cancer in a nucleic acid sample from the subject. The one or more polymorphic variations often are detected in or near the RAD21 nucleotide sequence, such as the nucleotide sequence set forth as SEQ ID NO: 1 or 2 or a substantially identical nucleotide sequence thereof.

Also featured are nucleic acids that encode a RAD21 polypeptide, and include one or more polymorphic variations associated with breast cancer, and oligonucleotides which hybridize to those nucleic acids. Also provided are polypeptides encoded by nucleic acids having a RAD21 nucleotide sequence, which include the full-length polypeptide, isoforms and fragments thereof. In addition, featured are methods for identifying candidate therapeutic molecules for treating breast cancer and related disorders, as well as methods of treating breast cancer in a subject by administering a therapeutic molecule.

Also provided are compositions comprising a breast cancer cell and/or a RAD21 nucleic acid, or a fragment or substantially identical nucleic acid thereof, with a RNAi, siRNA, antisense DNA or RNA, or ribozyme nucleic acid designed from a RAD21 nucleotide sequence. In an embodiment, the nucleic acid is designed from a RAD21 nucleotide sequence that includes one or more breast cancer associated polymorphic variations, and in some instances, specifically interacts with such a nucleotide sequence. Further, provided are arrays of nucleic acids bound to a solid surface, in which one or more nucleic acid molecules of the array are RAD21 nucleic acids, or a fragment or substantially identical nucleic acid thereof, or a complementary nucleic acid of the foregoing. Featured also are compositions comprising a breast cancer cell and/or a protein, polypeptide or peptide encoded by a RAD21 nucleic acid with an antibody that specifically binds to the protein, polypeptide or peptide. In an embodiment, the antibody specifically binds to an epitope in a RAD21 protein, polypeptide or peptide that includes a non-synonymous amino acid modification associated with breast cancer, such as a serine at position 656 of a NID2 protein, polypeptide or peptide.

A genomic RAD21 nucleotide sequence is set forth in FIGS. 1A-1S. In certain embodiments, a polymorphic variation at one or more positions in SEQ ID NO: 1 are detected from the group consisting of positions 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

In a related embodiment, provided are methods for treating breast cancer in a patient where RAD21 is downregulated by administering one or more anti-cancer agents or processes that cause strand breaks, such as single stranded breaks and/or double stranded breaks. In certain embodiments, the presence of a polymorphic variation associated with breast cancer is detected in a DNA sample from a subject before the agent is administered to the subject. In an embodiment, RAD21 is downregulated in the subject because the subject's DNA includes a RAD21 downregulating polymorphic variation. In another embodiment, the RAD21 is downregulated by administering an inhibitor of RAD21. Thus in some embodiments, a RAD21 inhibitor is administered in conjunction with an anticancer agent or process that causes strand breaks. Anticancer agents or processes that cause strand breaks include topoisomerase inhibitors, radiotherapy and radioimmunotherapy, for example. Examples of topoisomerase inhibitors (e.g., topoisomerase I or topoisomerase II inhibitors) include but are not limited to etoposide (VP16), doxorubicin (Adriamycin), camptothecin, irinotecan, topotecan, lurtotecan, exatecan, and teniposide). An agent (e.g., a RAD21 inhibitor and/or an agent that causes strand breaks) often are administered locally to a tumor, and often are administered before the tumor spreads so the agent will be able to treat the cancer during the time of rapid proliferation (i.e., the therapeutic window). In a related embodiment, provided are methods for identifying an increased or decreased response to anti-cancer agents that cause strand breaks, which comprise detecting the presence or absence of one or more polymorphic variations in a RAD21 nucleotide sequence that up or downregulates the expression of RAD21.

### BRIEF DESCRIPTION OF THE FIGURES

FIGS. 1A-1S show a genomic sequence of RAD21 with the polymorphic variants enclosed by brackets. The genomic sequence set forth in FIGS. 1A-1S corresponds to SEQ ID NO: 1. The following nucleotide representations are used throughout the specification and figures: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, G, or T; and “N” represents A, G, C, or T.

FIGS. 2A-2B show a human cDNA structure for RAD21. The cDNA sequence set forth in FIGS. 2A-2B corresponds to SEQ ID NO: 2.

FIG. 3 shows a human polypeptide sequences for RAD21. The polypeptide sequence set forth in FIG. 3 corresponds to SEQ ID NO: 3.

FIG. 4 shows proximal SNPs in the RAD21 protein. The position of each SNP on the chromosome is shown on the x-axis and the y-axis provides the negative logarithm of the p-value comparing the estimated allele to that of the control group. Also shown are exons and introns of the genes in the approximate chromosomal positions. The figure indicates that polymorphic variants associated with breast cancer in a region spanning positions 13191 to 33670 in SEQ ID NO: 1 are in linkage disequilibrium.

FIG. 5 shows a cumulative mRNA expression profile for RAD21 using a panel of 56 cells from different human tissues.

FIG. 6 shows relative levels of RAD21 mRNA in several breast cancer cell lines and in normal breast tissue.

FIG. 7 shows inhibition of RAD21 mRNA using several siRNAs that resulted in decreased proliferation and increased apoptosis in SQC-0049 (MCF-7) breast cancer cells.

FIG. 8 shows inhibition of RAD21 mRNA using several siRNAs that resulted in decreased proliferation and increased apoptosis in SQC-0080 (T47-D) breast cancer cells.

FIG. 9 shows the results of a clonogenic survival assay that demonstrates an increased sensitivity to bleomycin in cell lines where RAD21 expression is inhibited using siRNA.

FIG. 10 shows the results of a clonogenic survival assay that demonstrates an increased sensitivity to etoposide in cell lines where RAD21 expression is inhibited using siRNA.

### DETAILED DESCRIPTION

It has been discovered that polymorphic variants in or near a gene at chromosome position 8q24 encoding RAD21 (a homolog is present in S. pombe) are associated with increased risk of developing breast cancer in subjects. Genetic and biological data provided herein demonstrate RAD21 is intimately involved in breast cell proliferation by maintaining genomic stability during the cell cycle. RAD21 plays a role in DNA repair and in the proper segregation of chromatids during mitosis. The anti-proliferative effect of specific siRNAs on breast cancer cells likely is due to the loss of one or both of these two functions. Thus, detecting genetic determinants associated with an increased risk of breast cancer occurrence can lead to early identification of a risk of breast cancer and early prescription of preventative measures. Also, associating the polymorphic variants with breast cancer has provided a new target for diagnosing breast cancer and screening molecules useful in treatments of breast cancer. The additive effect of cellular exposure to DNA damaging chemotherapeutic agents and inhibition of RAD21 represents an attractive therapeutic approach for the treatment of breast cancer. Therefore, detecting genetic determinants associated with decreased RAD21 expression or exogenously inhibiting the expression of RAD21 in conjunction with the administration of DNA damaging chemotherapeutic agents offers a novel, improved method for treating breast cancer.

Breast Cancer and Sample Selection

Breast cancer is typically described as the uncontrolled growth of malignant breast tissue. Breast cancers arise most commonly in the lining of the milk ducts of the breast (ductal carcinoma), or in the lobules where breast milk is produced (lobular carcinoma). Other forms of breast cancer include Inflammatory Breast Cancer and Recurrent Breast Cancer. Inflammatory breast cancer is a rare, but very serious, aggressive type of breast cancer. The breast may look red and feel warm with ridges, welts, or hives on the breast; or the skin may look wrinkled. It is sometimes misdiagnosed as a simple infection. Recurrent disease means that the cancer has come back after it has been treated. It may come back in the breast, in the soft tissues of the chest (the chest wall), or in another part of the body.

As used herein, the term “breast cancer” refers to a condition characterized by anomalous rapid proliferation of abnormal cells in one or both breasts of a subject. The abnormal cells often are referred to as “neoplastic cells,” which are transformed cells that can form a solid tumor. The term “tumor” refers to an abnormal mass or population of cells (i.e. two or more cells) that result from excessive or abnormal cell division, whether malignant or benign, and pre-cancerous and cancerous cells. Malignant tumors are distinguished from benign growths or tumors in that, in addition to uncontrolled. cellular proliferation, they can invade surrounding tissues and can metastasize. In breast cancer, neoplastic cells may be identified in one or both breasts only and not in another tissue or organ, in one or both breasts and one or more adjacent tissues or organs (e.g. lymph node), or in a breast and one or more non-adjacent tissues or organs to which the breast cancer cells have metastasized.

The term “invasion” as used herein refers to the spread of cancerous cells to adjacent surrounding tissues. The term “metastasis” as used herein refers to a process in which cancer cells travel from one organ or tissue to another non-adjacent organ or tissue. Cancer cells in the breast(s) can spread to tissues and organs of a subject, and conversely, cancer cells from other organs or tissue can invade or metastasize to a breast. Cancerous cells from the breast(s) may invade or metastasize to any other organ or tissue of the body. Breast cancer cells often invade lymph node cells and/or metastasize to the liver, brain and/or bone and spread cancer in these tissues and organs. Breast cancers can spread to other organs and tissues and cause lung cancer, prostate cancer, colon cancer, ovarian cancer, cervical cancer, gastrointestinal cancer, pancreatic cancer, glioblastoma, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, breast cancer, ovarian cancer, neuroblastoma, myeloma, various types of head and neck cancer, acute lymphoblastic leukemia, acute myeloid leukemia, Ewing sarcoma and peripheral neuroepithelioma, and other carcinomas, lymphomas, blastomas, sarcomas, and leukemias.

In an effort to detect breast cancer as early as possible, regular physical exams and screening mammograms often are prescribed and conducted. A diagnostic mammogram often is performed to evaluate a breast complaint or abnormality detected by physical exam or routine screening mammography. If an abnormality seen with diagnostic mammography is suspicious, additional breast imaging (with exams such as ultrasound) or a biopsy may be ordered. A biopsy followed by pathological (microscopic) analysis is a definitive way to determine whether a subject has breast cancer. Excised breast cancer samples often are subjected to the following analyses: diagnosis of the breast tumor and confirmation of its malignancy; maximum tumor thickness; assessment of completeness of excision of invasive and in situ components and microscopic measurements of the shortest extent of clearance; level of invasion; presence and extent of regression; presence and extent of ulceration; histological type and special variants; pre-existing lesion; mitotic rate; vascular invasion; neurotropism; cell type; tumor lymphocyte infiltration; and growth phase.

The stage of a breast cancer can be classified as a range of stages from Stage 0 to Stage IV based on its size and the extent to which it has spread. The following table summarizes the stages:

TABLE 1

| | | | Metastasis |
|---|---|---|---|
| Stage | Tumor Size | Lymph Node Involvement | (Spread) |
| I | Less than 2 cm | No | No |
| II | Between 2-5 cm | No or in same side of breast | No |
| III | More than 5 cm | Yes, on same side of breast | No |
| IV | Not applicable | Not applicable | Yes |

Stage 0 cancer is a contained cancer that has not spread beyond the breast ductal system. Fifteen to twenty percent of breast cancers detected by clinical examinations or testing are in Stage 0 (the earliest form of breast cancer). Two types of Stage 0 cancer are lobular carcinoma in situ (LCIS) and ductal carcinoma in situ (DCIS). LCIS indicates high risk for breast cancer. Many physicians do not classify LCIS as a malignancy and often encounter LCIS by chance on breast biopsy while investigating another area of concern. While the microscopic features of LCIS are abnormal and are similar to malignancy, LCIS does not behave as a cancer (and therefore is not treated as a cancer). LCIS is merely a marker for a significantly increased risk of cancer anywhere in the breast. However, bilateral simple mastectomy may be occasionally performed if LCIS patients have a strong family history of breast cancer. In DCIS the cancer cells are confined to milk ducts in the breast and have not spread into the fatty breast tissue or to any other part of the body (such as the lymph nodes). DCIS may be detected on mammogram as tiny specks of calcium (known as microcalcifications) 80% of the time. Less commonly DCIS can present itself as a mass with calcifications (15% of the time); and even less likely as a mass without calcifications (<5% of the time). Breast biopsy is used to confirm DCIS. Standard DCIS treatment is breast-conserving therapy (BCT): lumpectomy followed by radiation treatment or mastectomy. To date, DCIS patients have chosen equally among lumpectomy and mastectomy as their treatment option, though specific cases may sometimes favor lumpectomy over mastectomy or vice versa.

In Stage I, the primary (original) cancer is 2 cm or less in diameter and has not spread to the lymph nodes. In Stage IIA, the primary tumor is between 2 and 5 cm in diameter and has not spread to the lymph nodes. In Stage IIB, the primary tumor is between 2 and 5 cm in diameter and has spread to the axillary (underarm) lymph nodes; or the primary tumor is over 5 cm and has not spread to the lymph nodes. In Stage IIIA, the primary breast cancer of any kind that has spread to the axillary (underarm) lymph nodes and to axillary tissues. In Stage IIIB, the primary breast cancer is any size, has attached itself to the chest wall, and has spread to the pectoral (chest) lymph nodes. In Stage IV, the primary cancer has spread out of the breast to other parts of the body (such as bone, lung, liver, brain). The treatment of Stage IV breast cancer focuses on extending survival time and relieving symptoms.

Based in part upon selection criteria set forth above, individuals having breast cancer can be selected for genetic studies. Also, individuals having no history of cancer or breast cancer often are selected for genetic studies. Other selection criteria can include: a tissue or fluid sample is derived from an individual characterized as Caucasian; the sample was derived from an individual of German paternal and maternal descent; the database included relevant phenotype information for the individual; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Phenotype information included pre- or post-menopausal, familial predisposition, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), method of treatment (surgery, chemotherapy, hormone therapy, radiation therapy).

Provided herein is a set of blood samples and a set of corresponding nucleic acid samples isolated from the blood samples, where the blood samples are donated from individuals diagnosed with breast cancer. The sample set often includes blood samples or nucleic acid samples from 100 or more, 150 or more, or 200 or more individuals having breast cancer, and sometimes from 250 or more, 300 or more, 400 or more, or 500 or more individuals. The individuals can have parents from any place of origin, and in an embodiment, the set of samples are extracted from individuals of German paternal and German maternal ancestry. The samples in each set may be selected based upon five or more criteria and/or phenotypes set forth above.

Polymorphic Variants Associated with Breast Cancer

A genetic analysis provided herein linked breast cancer with polymorphic variant nucleic acid sequences in the human genome. As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

Furthermore, a genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, the term “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of breast cancer.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein, specifically in Example 2. A polymorphic variant is statistically significant and often biologically relevant if it is represented in 5% or more of a population, sometimes 10% or more, 15% or more, or 20% or more of a population, and often 25% or more, 30% or more, 35% or more, 40% or more, 45% or more, or 50% or more of a population.

A polymorphic variant may be detected on either or both strands of a double-stranded nucleic acid. Also, a polymorphic variant may be located within an intron or exon of a gene or within a portion of a regulatory region such as a promoter, a 5′ untranslated region (UTR), a 3′ UTR, and in DNA (e.g., genomic DNA (GDNA) and complementary DNA (cDNA)), RNA (e.g., mRNA, tRNA, and rRNA), or a polypeptide. Polymorphic variations may or may not result in detectable differences in gene expression, polypeptide structure, or polypeptide function.

In the genetic analysis that associated breast cancer with the polymorphic variants set forth in Table 9, samples from individuals having breast cancer and individuals not having cancer were allelotyped and genotyped. The term “allelotype” as used herein refers to a process for determining the allele frequency for a polymorphic variant in pooled DNA samples from cases and controls. By pooling DNA from each group, an allele frequency for each SNP in each group is calculated. These allele frequencies are then compared to one another. Particular SNPs are considered as being associated with a particular disease when allele frequency differences calculated between case and control pools are statistically significant. The term “genotyped” as used herein refers to a process for determining a genotype of one or more individuals, where a “genotype” is a representation of one or more polymorphic variants in a population.

Furthermore, a genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

It has been discovered that a polymorphic variation in the RAD21 gene is associated with the occurrence of breast cancer. Thus, featured herein are methods for identifying a risk of breast cancer in a subject, which comprises detecting the presence or absence of one or more of the polymorphic variations described herein in a human nucleic acid sample. The original polymorphic variation (or incident SNP) is described in Table 2 below:

TABLE 2

| | | | | | | | Diabetes |
|---|---|---|---|---|---|---|---|
| SNP | Chromosome | Contig | Contig | Sequence | Sequence | Allelic | Associated |
| Reference | Position | Identification | Position | Identification | Position | Variability | Allele |
| rs1374297 | 117553494 | NT_008046 | 31102874 | NM_006265 | intragenic | G/C | G |

Table 2 includes information pertaining to an incident polymorphic variant associated with breast cancer identified herein. Public information pertaining to the polymorphism and the genomic sequence that includes the polymorphism are indicated. The genomic sequence identified in Table 2 may be accessed at the http address www.ncbi.nih.gov/entrez/query.fcgi, for example, by using the publicly available SNP reference number (e.g., rs1374297). The chromosome position refers to the position of the SNP within NCBI's Genome build 33, which may be accessed at the following http address: www.ncbi.nlm.nih.gov/mapview/map_search.cgi?chr=hum_chr.inf&query=. The “Contig Position” provided in Table 2 corresponds to a nucleotide position set forth in the contig sequence, and designates the polymorphic site corresponding to the SNP reference number. The sequence containing the polymorphisms also may be referenced by the “Sequence Identification” set forth in Table 2. The “Sequence Identification” corresponds to cDNA sequence that encodes associated polypeptides (e.g., RAD21). The position of the SNP within the cDNA sequence is provided in the “Sequence Position” column of Table 2. Also, the allelic variation at the polymorphic site and the allelic variant identified as associated with breast cancer is specified in Table 2. The alleles provided in the allelic variability column in Table 2 and all subsequent tables and figures may not be in the same orientation (e.g., forward or reverse) as the reference sequence. All nucleotide sequences referenced and accessed by the parameters set forth in Table 2 are incorporated herein by reference.

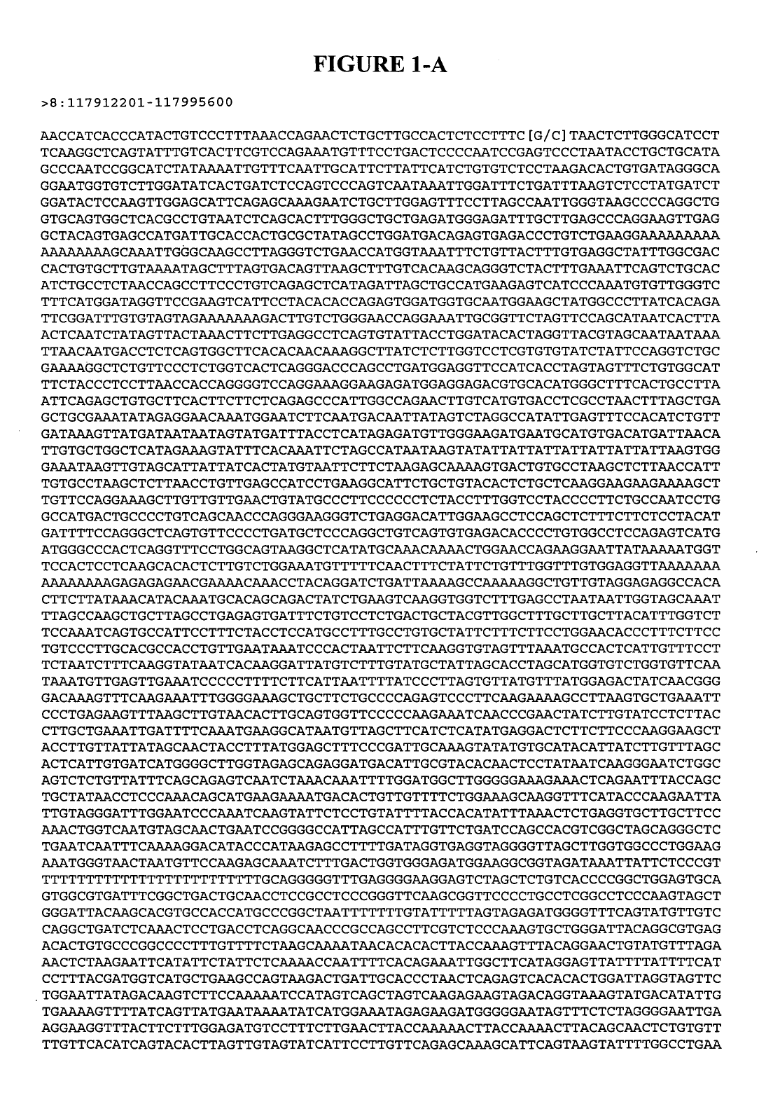

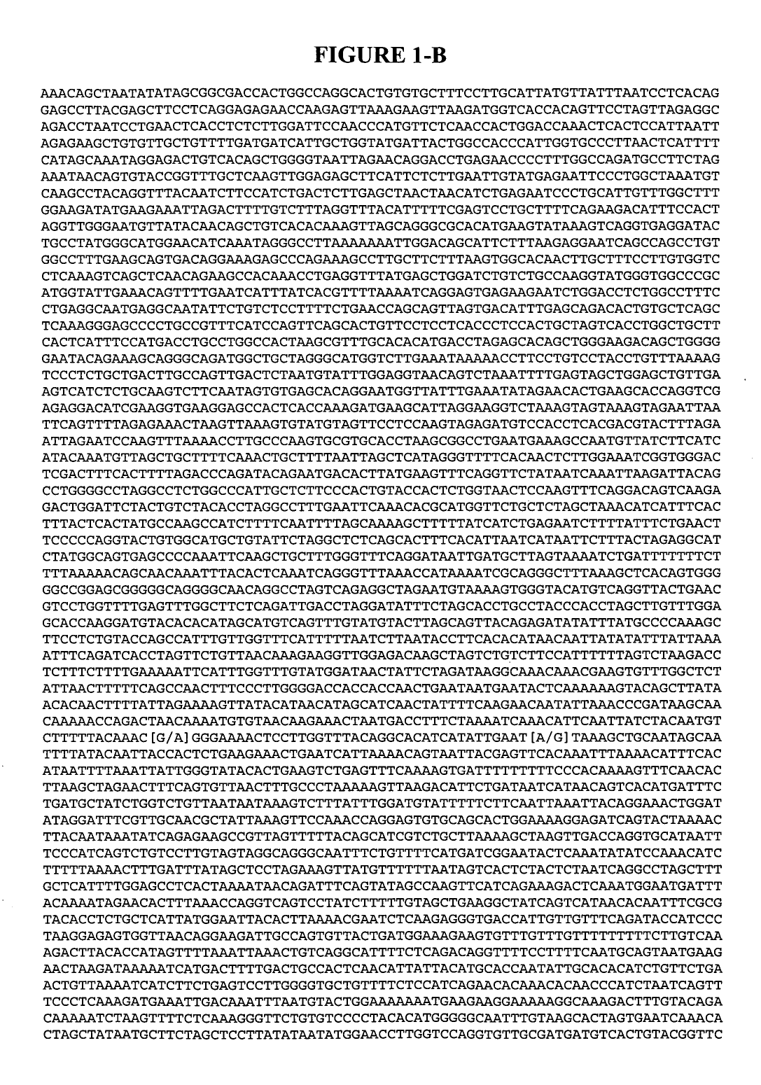

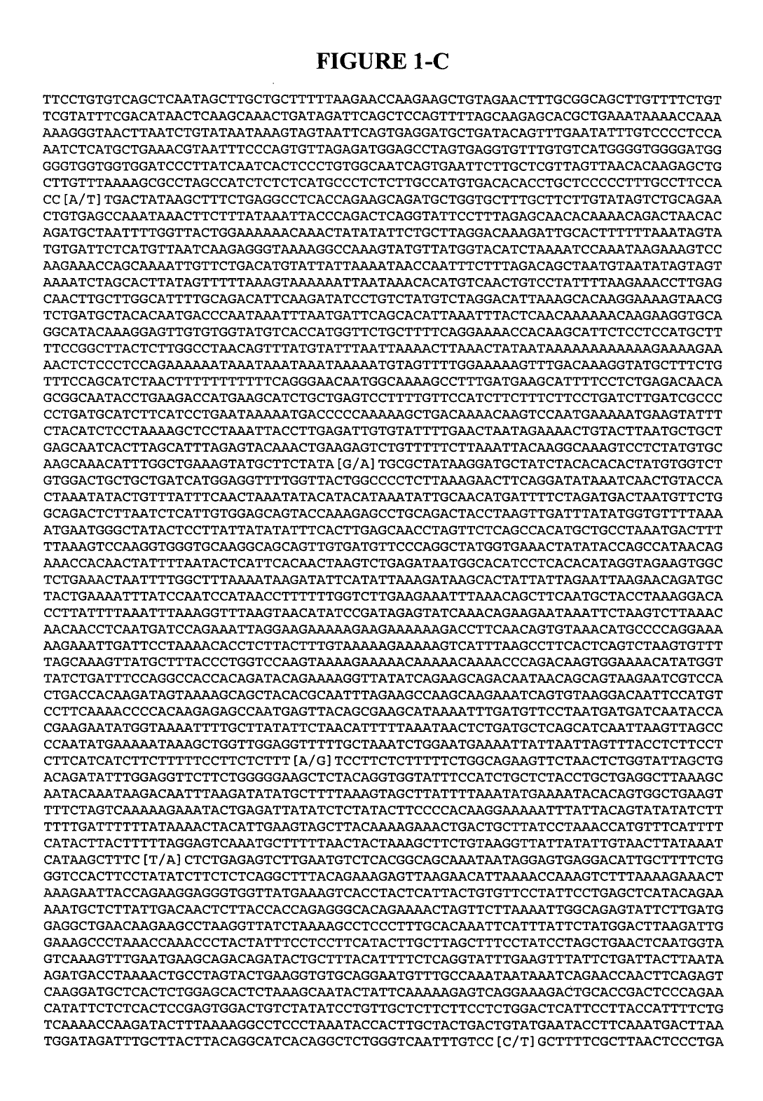

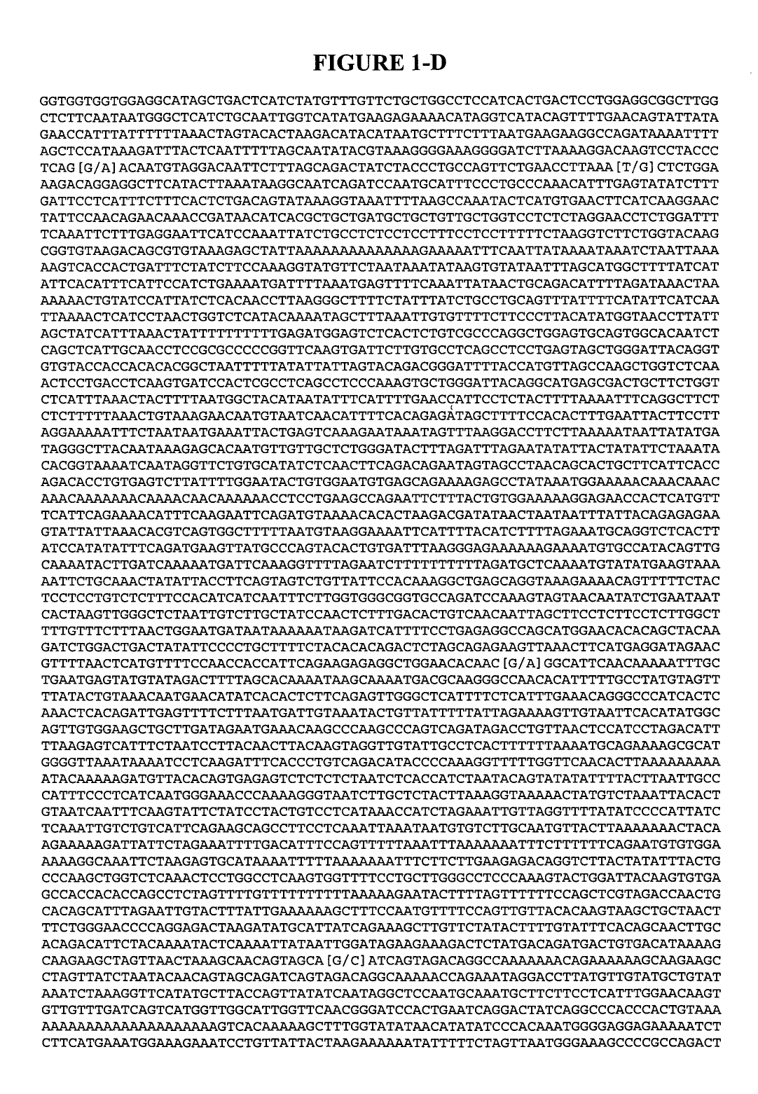

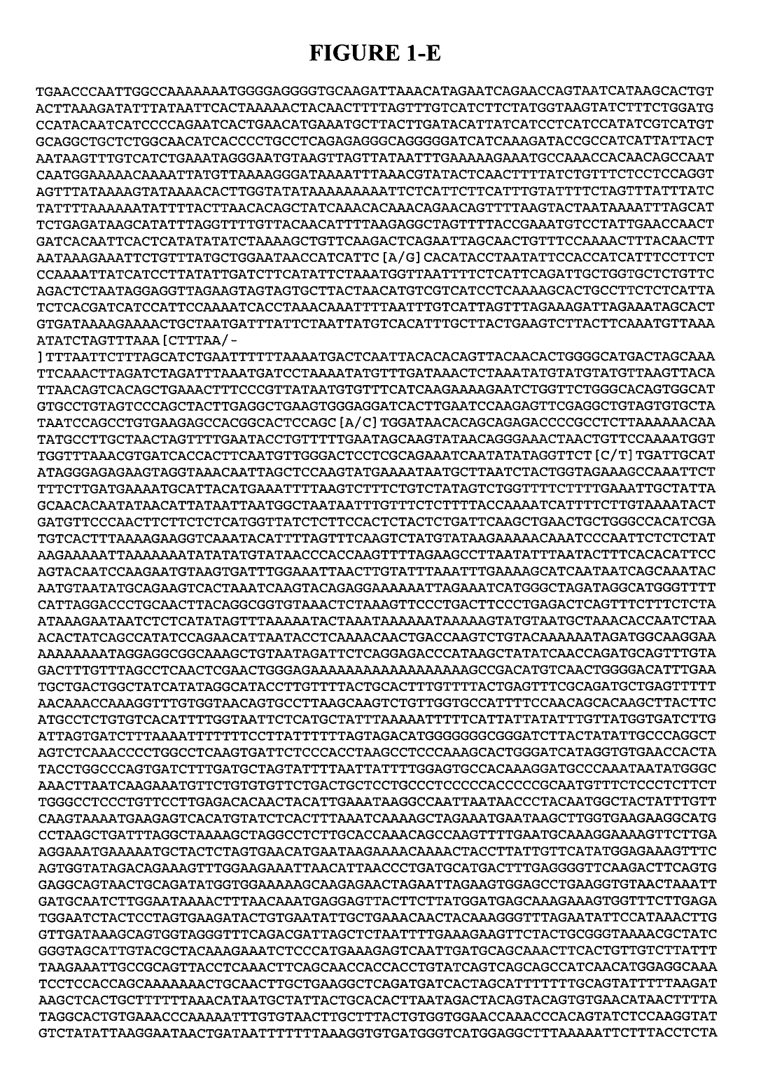

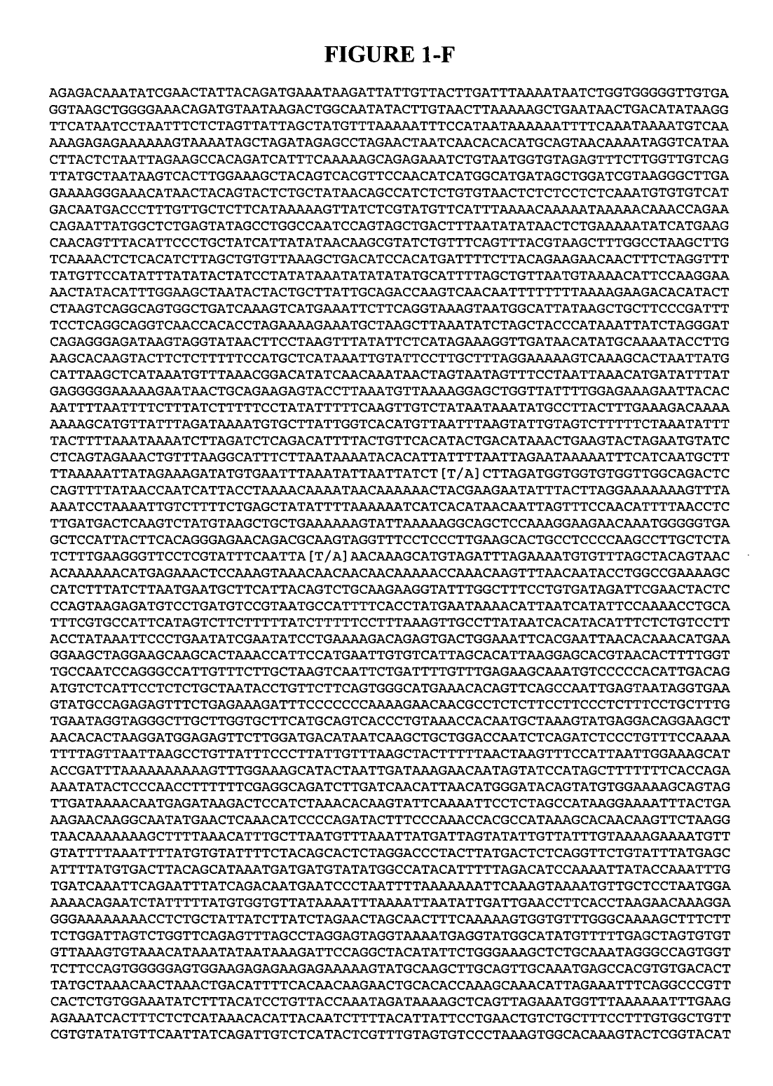

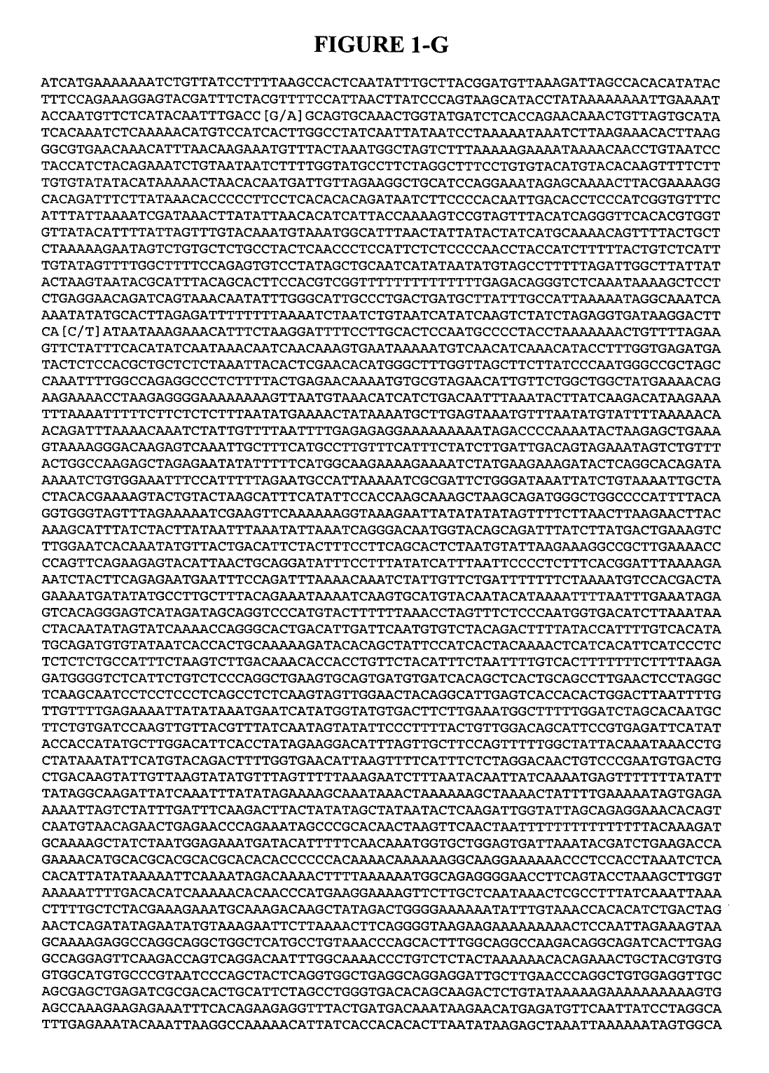

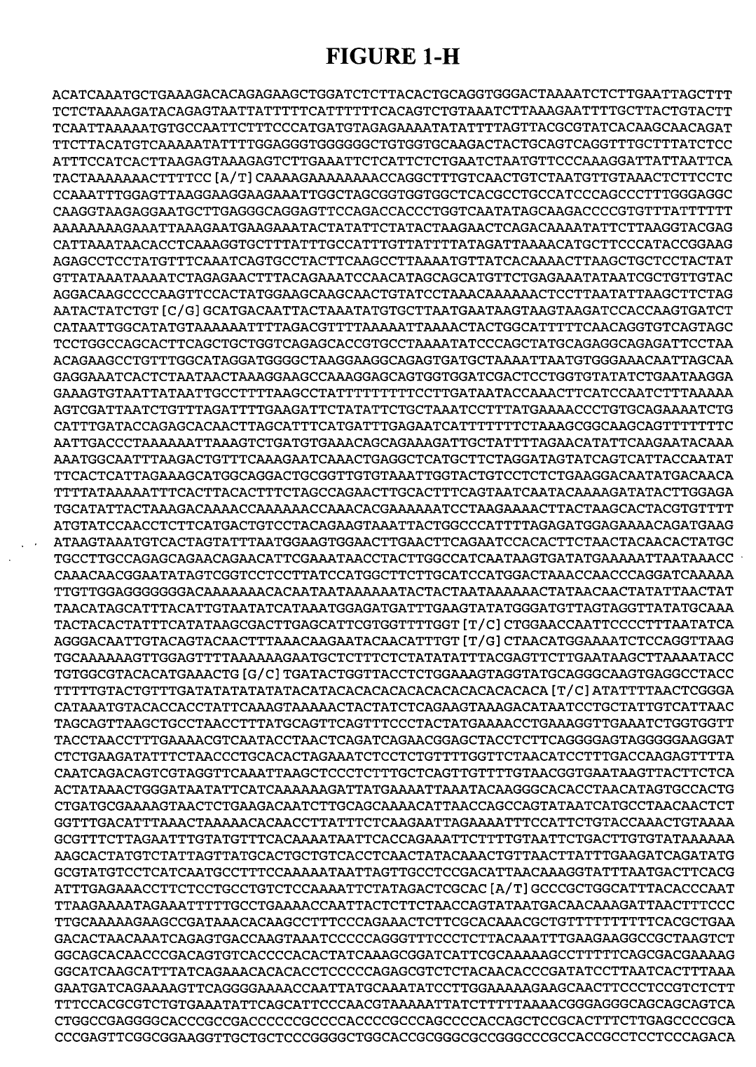

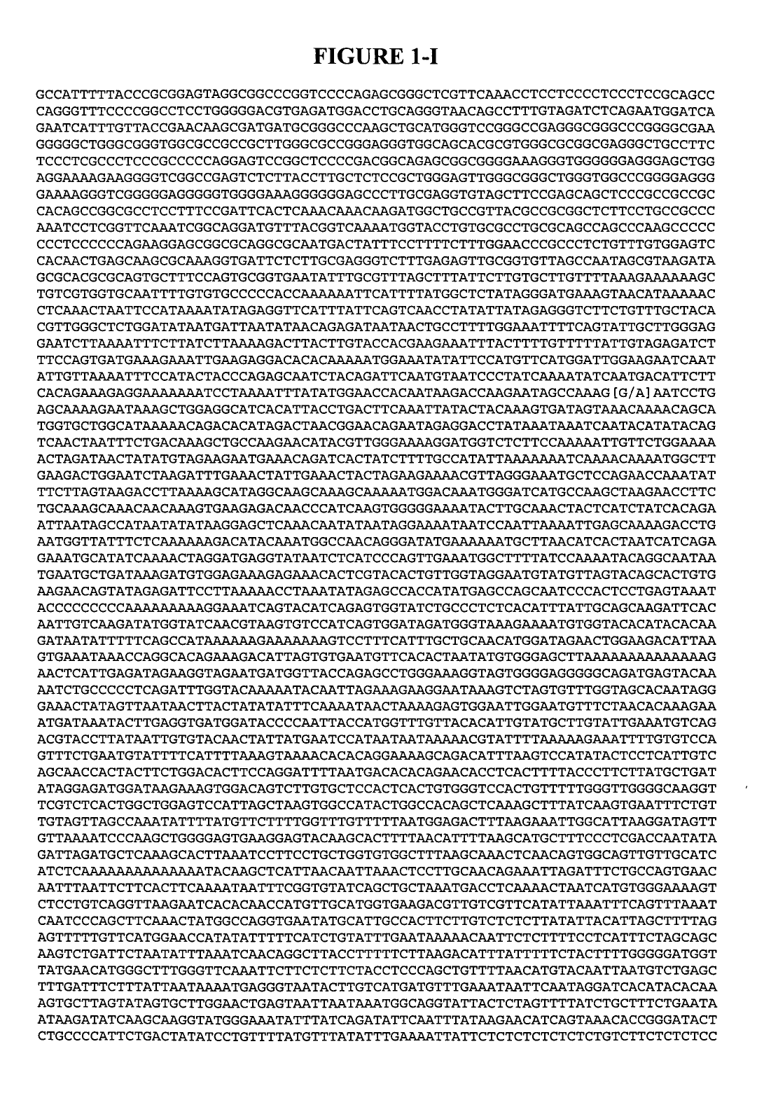

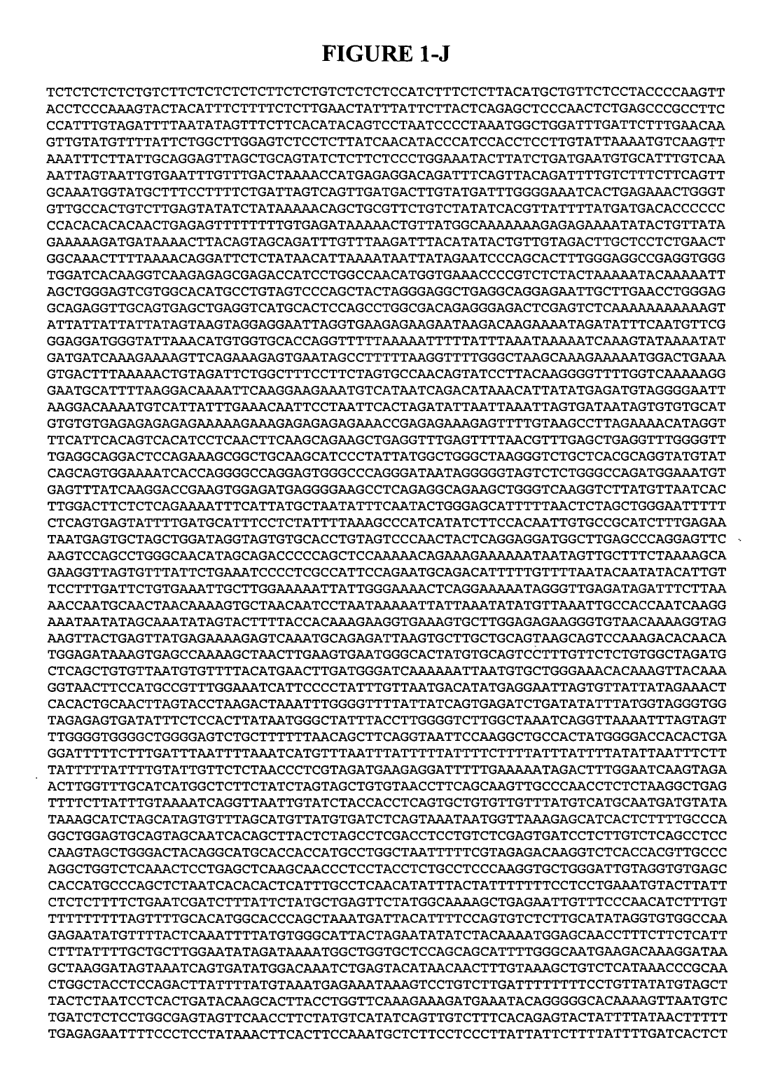

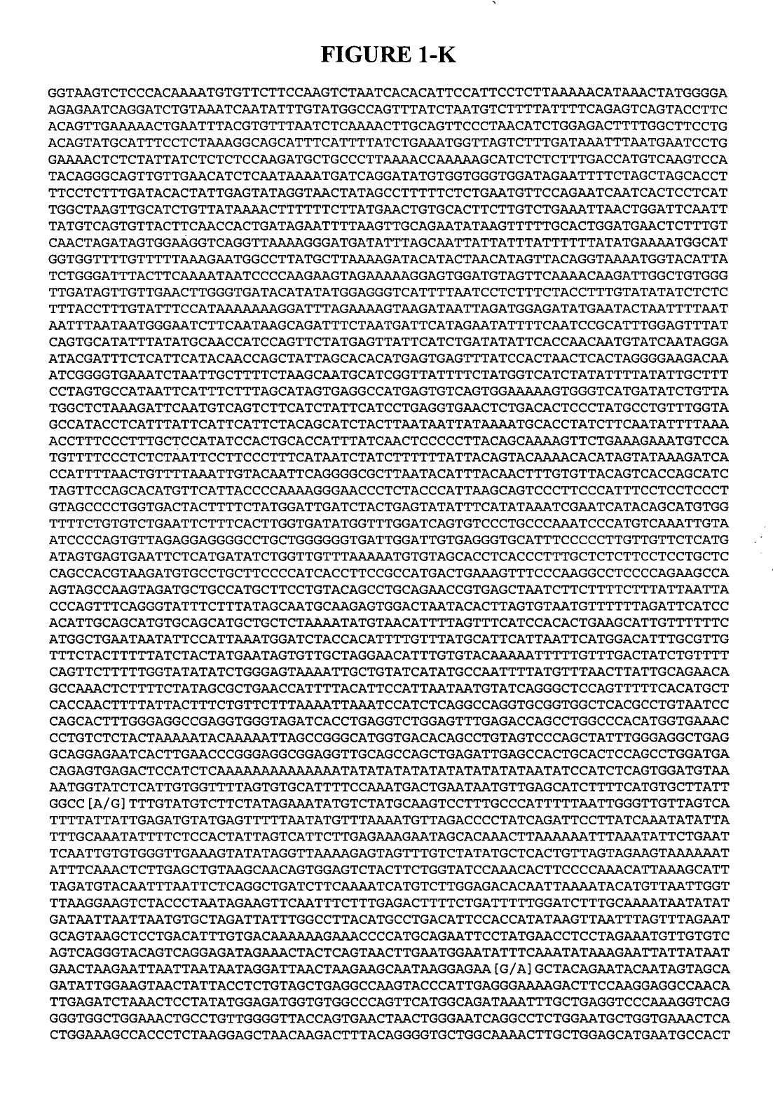

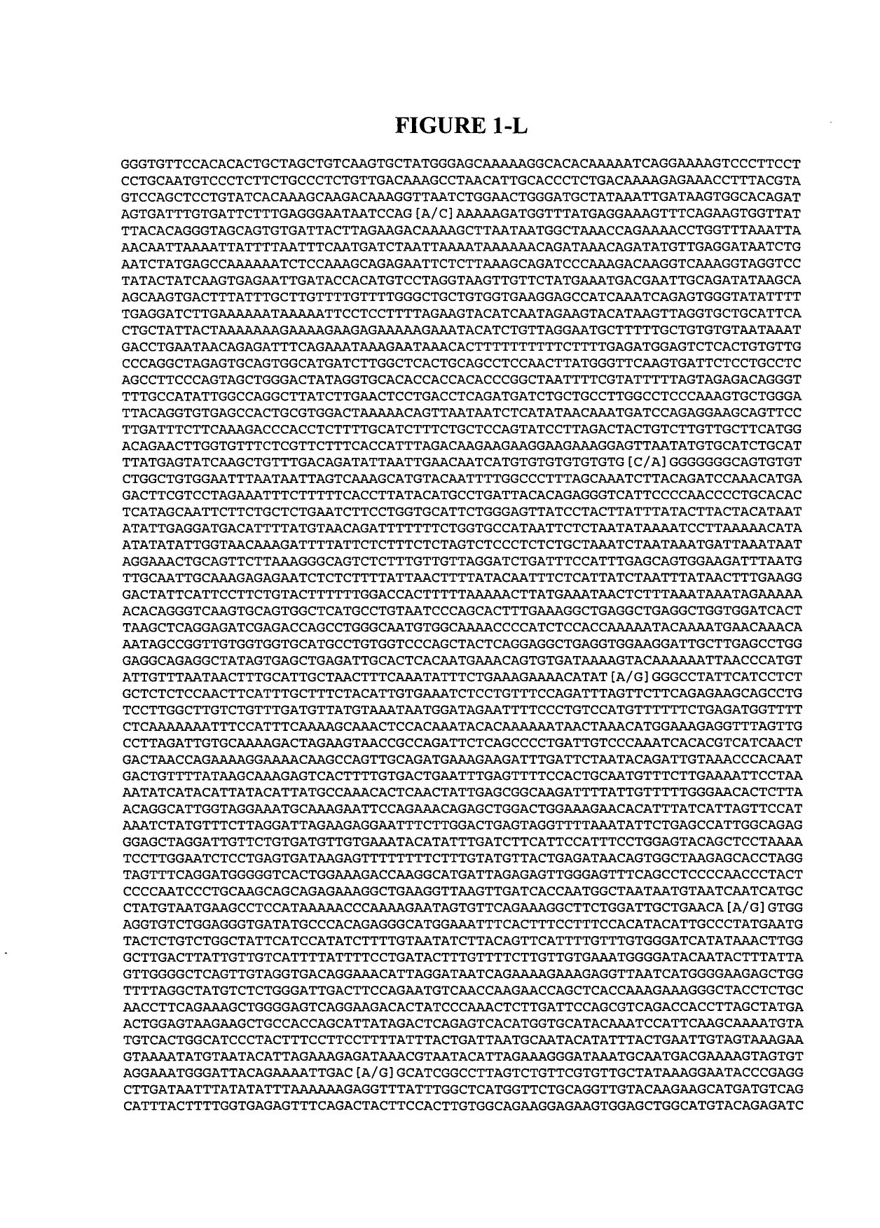

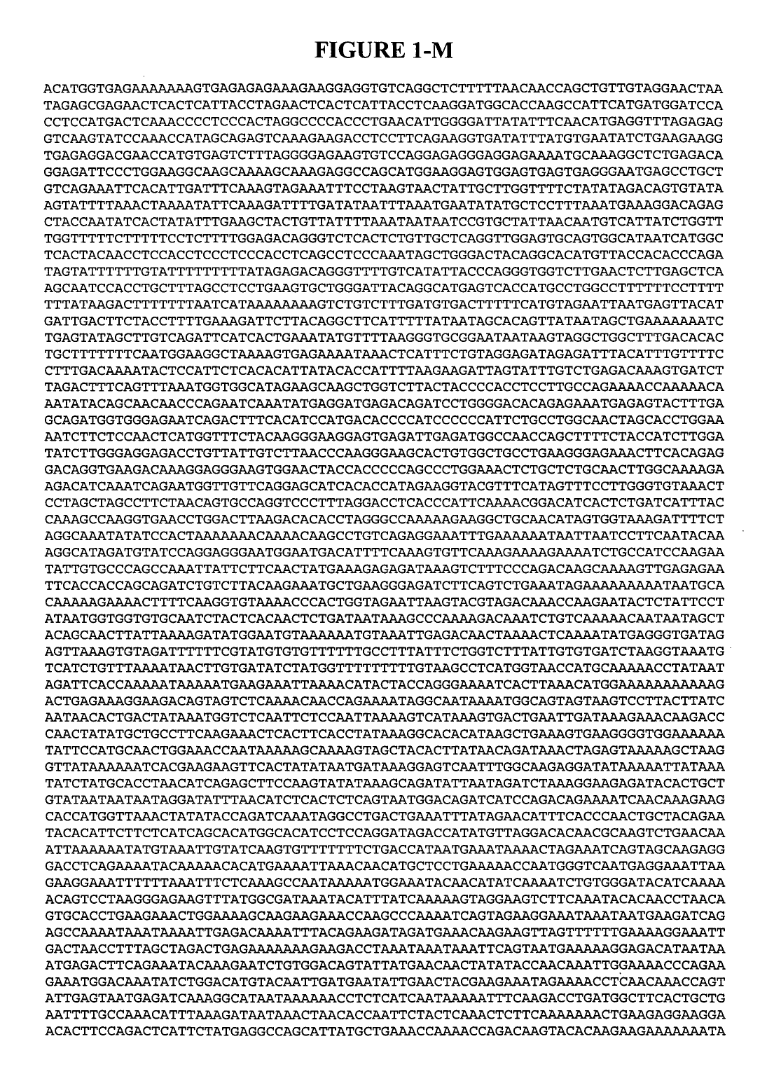

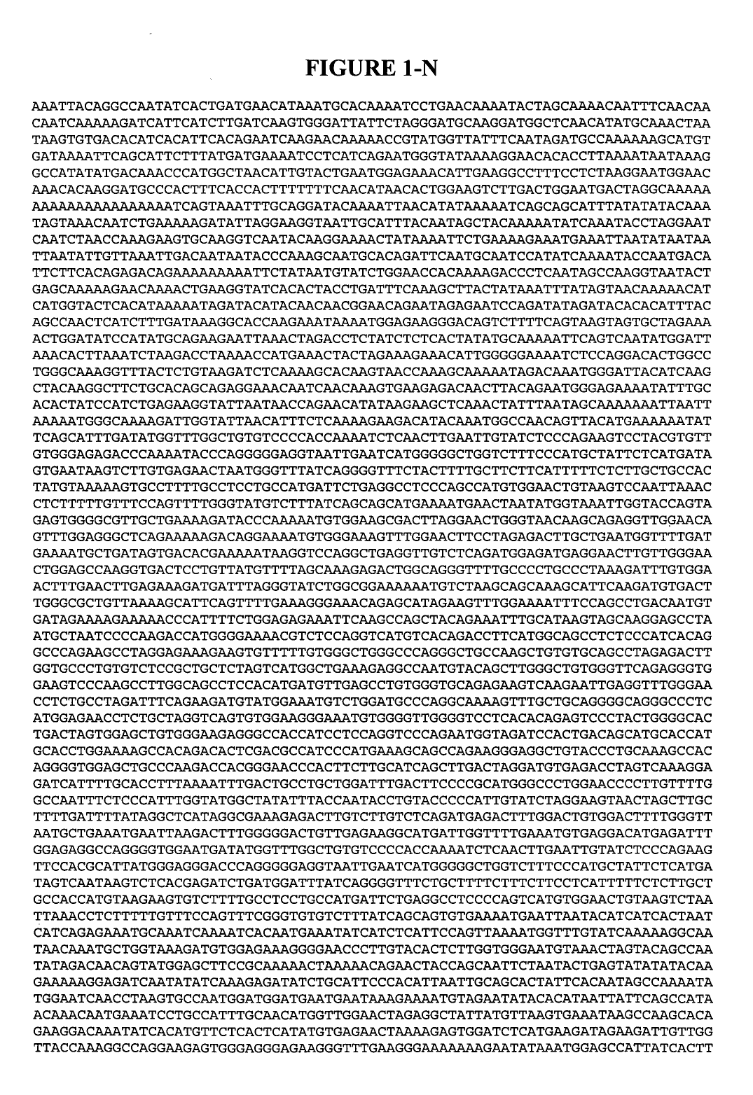

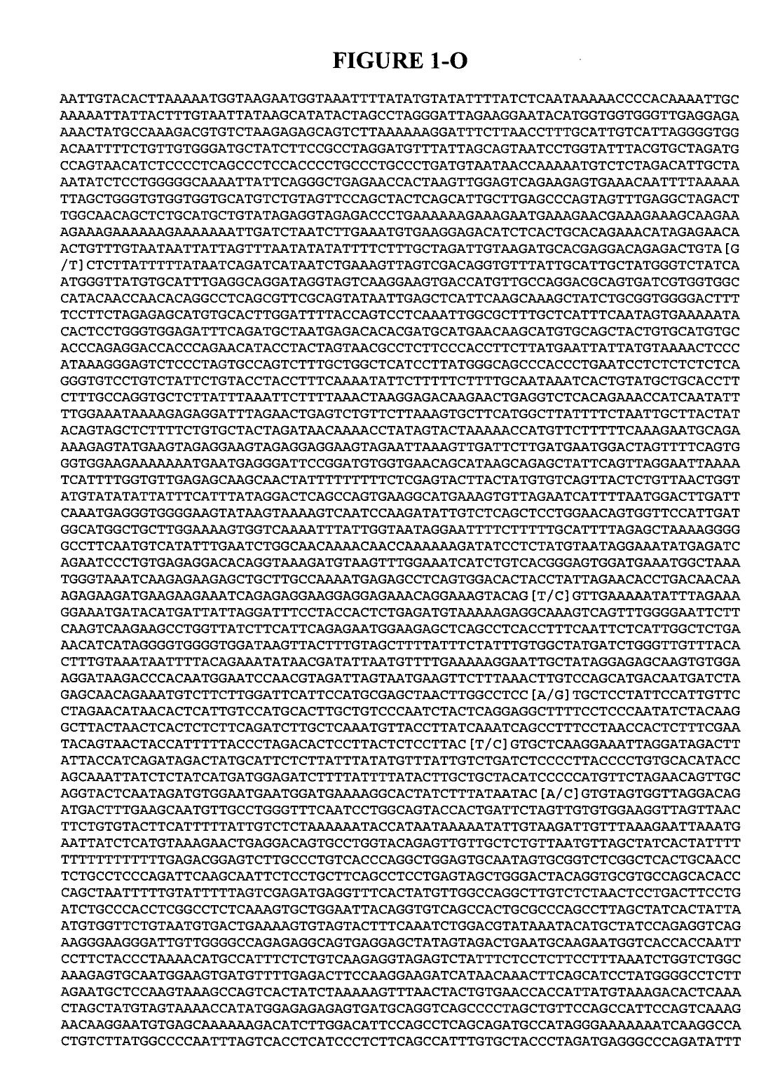

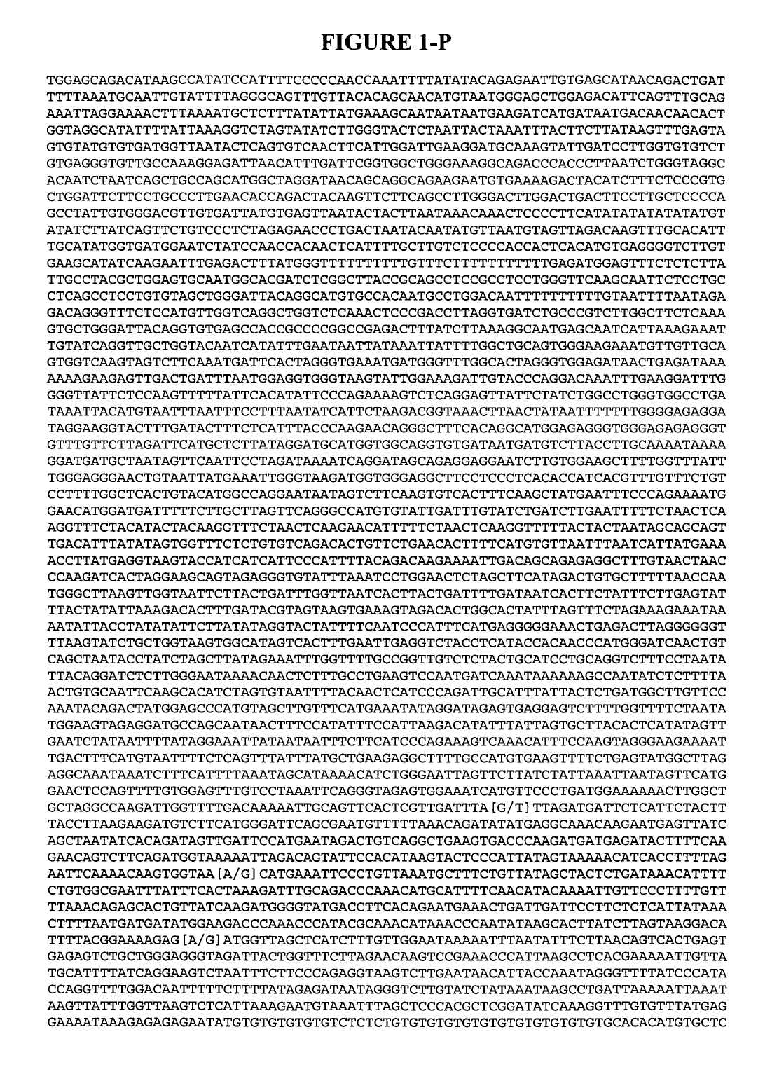

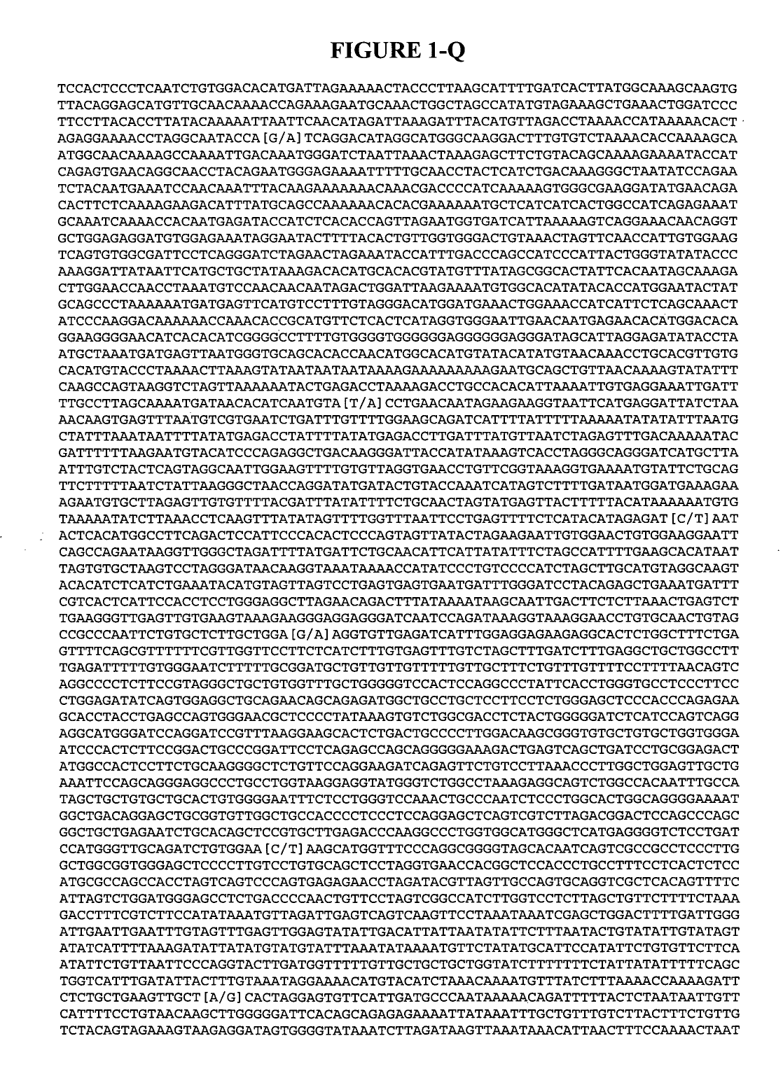

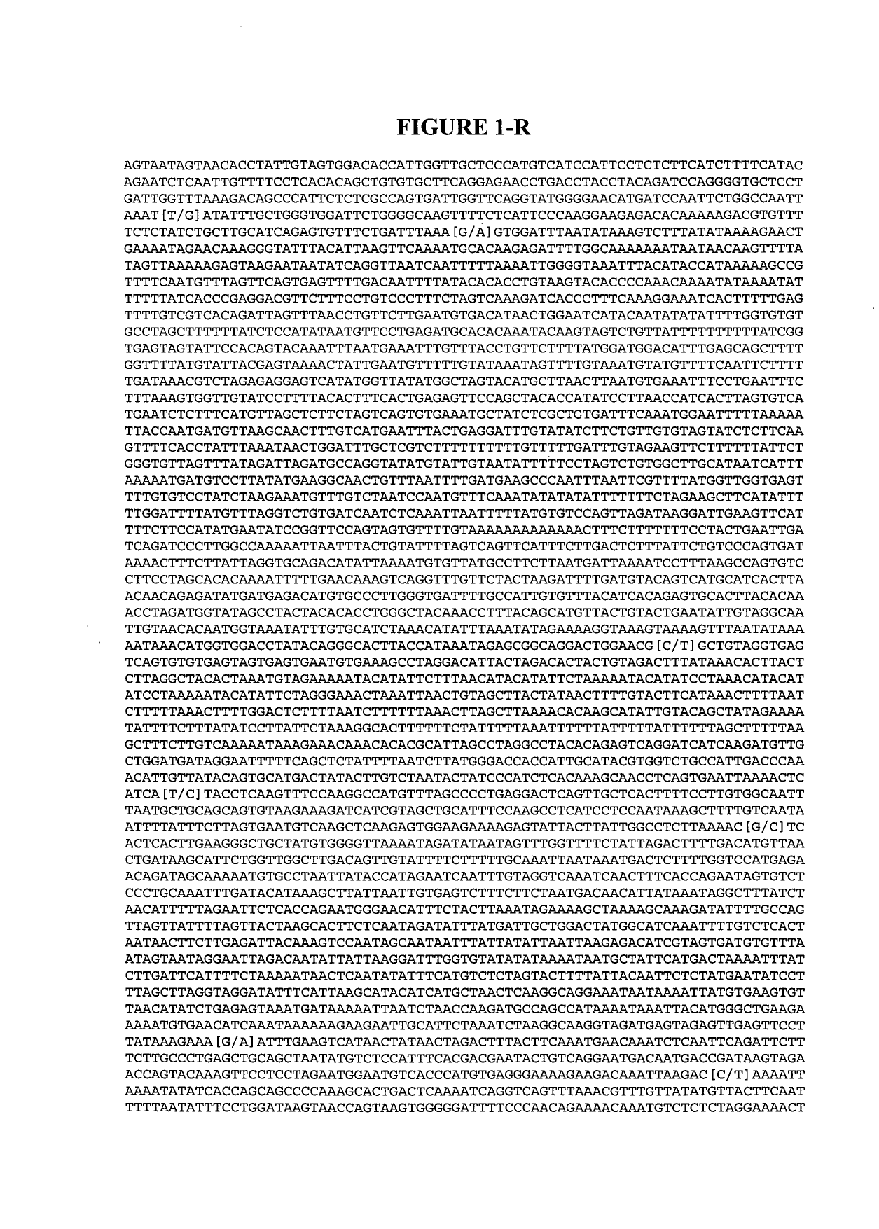

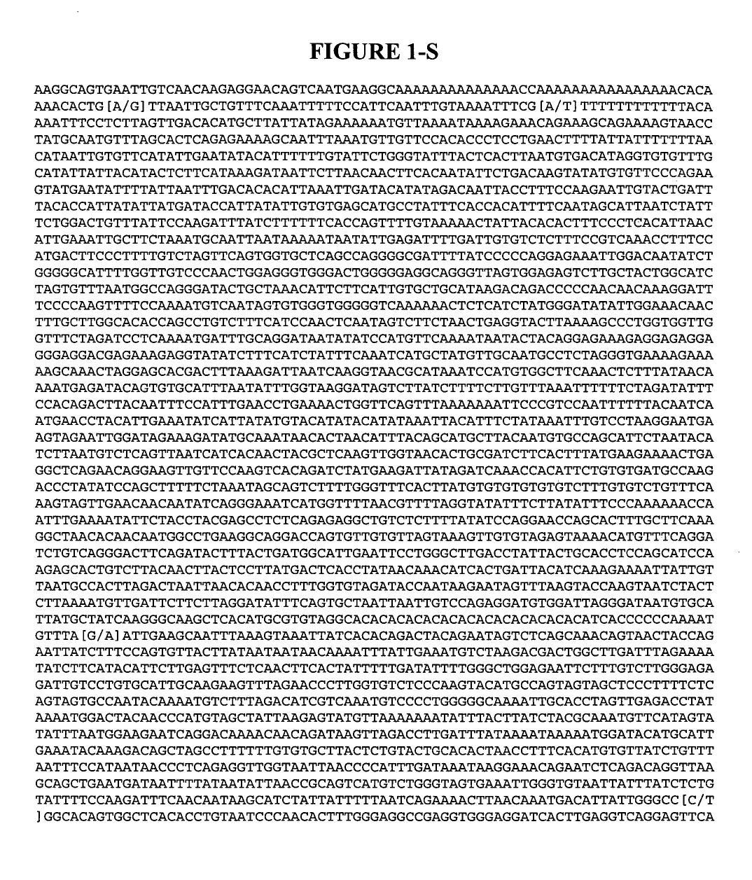

SNPs at the following chromosome positions were identified proximal to the incident SNP and subsequently allelotyped in the case and control populations as described herein: 117912256, 117919524, 117919563, 117921431, 117922690, 117924067, 117924508, 117925391, 117925725, 117925782, 117928075, 117929437, 117930672, 117931370, 117931549, 117936069, 117936435, 117938891, 117943573, 117944179, 117945672, 117945745, 117945870, 117945978, 117946891, 117949059, 117959851, 117960663, 117961258, 117962433, 117963428, 117964515, 117965239, 117975140, 117976731, 117977189, 117977409, 117977644, 117982256, 117982529, 117982829, 117983526, 117984763, 117985335, 117985827, 117986821, 117987503, 117987949, 117988055, 117989999, 117990632, 117990848, 117991785, 117991991, 117992237, 117992282, 117994690 or 117995524, as set forth in Table 9. Alternatively, the proximal SNPs disclosed herein are also set forth in FIG. 1 at the following positions: 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 or 83324.

Additional Polymorphic Variants Associated with Breast Cancer

Also provided is a method for identifying polymorphic variants proximal to an incident, founder polymorphic variant associated with breast cancer. Thus, featured herein are methods for identifying a polymorphic variation associated with breast cancer that is proximal to an incident polymorphic variation associated with breast cancer, which comprises identifying a polymorphic variant proximal to the incident polymorphic variant associated with breast cancer, where the incident polymorphic variant is in a nucleotide sequence set forth in SEQ ID NO: 1 or 2. The nucleotide sequence often comprises a polynucleotide sequence selected from the group consisting of (a) a polynucleotide sequence set forth in SEQ ID NO: 1 or 2; (b) a polynucleotide sequence that encodes a polypeptide having an amino acid sequence encoded by a nucleotide sequence set forth in SEQ ID NO: 1 or 2; and (c) a polynucleotide sequence that encodes a polypeptide having an amino acid sequence that is 90% or more identical to an amino acid sequence encoded by a nucleotide sequence set forth in SEQ ID NO: 1 or 2 or a polynucleotide sequence 90% or more identical to the polynucleotide sequence set forth in SEQ ID NO: 1 or 2. The presence or absence of an association of the proximal polymorphic variant with breast cancer then is determined using a known association method, such as a method described in the Examples hereafter. In an embodiment, the incident polymorphic variant is described in SEQ ID NO: 1 or 2 or Table 9. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic samples. Thus, multiple polymorphic variants proximal to an incident polymorphic variant are associated with breast cancer using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3′ of the incident polymorphic variant.

In certain embodiments, polymorphic variants associated with breast cancer are identified iteratively. For example, a first proximal polymorphic variant is associated with breast cancer using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with breast cancer is determined.

The methods described herein are useful for identifying or discovering additional polymorphic variants that may be used to further characterize a gene, region or loci associated with a condition, a disease (e.g., breast cancer), or a disorder. For example, allelotyping or genotyping data from the additional polymorphic variants may be used to identify a functional mutation or a region of linkage disequilibrium (see Example 3).

In certain embodiments, polymorphic variants identified or discovered within a region comprising the first polymorphic variant associated with breast cancer are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with breast cancer, and such information can be used in prognosis methods described herein.

Isolated Nucleic Acids

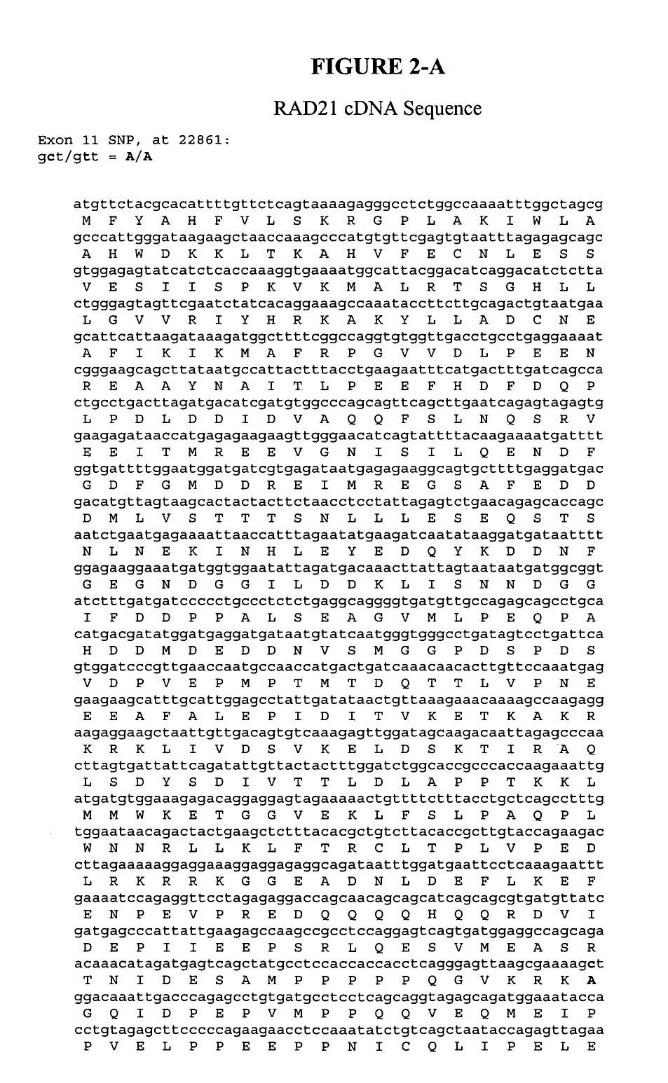

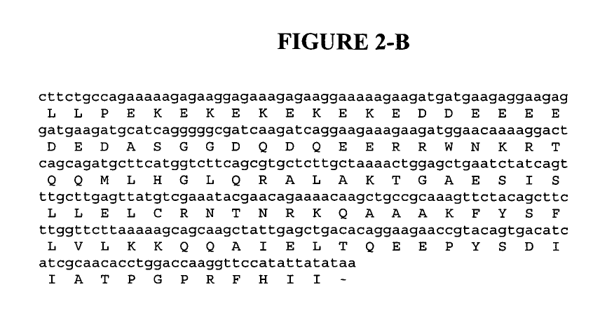

Featured herein are isolated nucleic acid variants depicted in FIGS. 1 and 2, and substantially identical nucleic acids thereof. A nucleic acid variant may be represented on one or both strands in a double-stranded nucleic acid or on one chromosomal complement (heterozygous) or both chromosomal complements (homozygous).

As used herein, the term “nucleic acid” includes DNA molecules (e.g., a complementary DNA (cDNA) and genomic DNA (gDNA)) and RNA molecules (e.g., mRNA, rRNA, siRNA and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “gene” refers to a nucleotide sequence that encodes a polypeptide.

Also included herein are nucleic acid fragments. These fragments often are a nucleotide sequence identical to a nucleotide sequence in FIGS. 1A-1S or FIG. 2, a nucleotide sequence substantially identical to a nucleotide sequence in FIGS. 1A-1S or FIG. 2, or a nucleotide sequence that is complementary to the foregoing. The nucleic acid fragment may be identical, substantially identical or homologous to a nucleotide sequence in an exon or an intron in a nucleotide sequence of FIGS. 1A-1S or FIG. 2. Further, the nucleic acid fragment may encode a full-length or mature polypeptide, or may encode a domain or part of a domain of a polypeptide. RAD21 domains include, but are not limited to, transmembrane domains (520-591, 679-732, 952-1023, 1126-1179 base pairs of FIG. 2), the protease domain (928-1461 base pairs of FIG. 2), and the Zn-binding motif (HEXXH) present in the protease domain (1075-1089 base pairs of FIG. 2). Sometimes, the fragment will comprises one or more of the polymorphic variations described herein as being associated with breast cancer. The nucleic acid fragment is often 50, 100, or 200 or fewer base pairs in length, and is sometimes about 300, 400, 500, 600, 700, 800, 900, 1000, 1100, 1200, 1300, 1400, 1500, 2000, 3000, 4000, 5000, 10000, 15000, or 20000 base pairs in length. A nucleic acid fragment that is complementary to a nucleotide sequence identical or substantially identical to a nucleotide sequence in FIGS. 1A-1S or FIG. 2 and hybridizes to such a nucleotide sequence under stringent conditions is often referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of prognostic or diagnostic assays, for example, as described herein.

Oligonucleotides are typically synthesized using standard methods and equipment, such as the ABI™3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides may also be linked to a second moiety. The second moiety may be an additional nucleotide sequence such as a tail sequence (e.g., a polyadenosine tail), an adapter sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

Uses for Nucleic Acid Sequences

Nucleic acid coding sequences depicted in FIGS. 1A-1S or FIG. 2 may be used for diagnostic purposes for detection and control of polypeptide expression. Also, included herein are oligonucleotide sequences such as antisense RNA, small-interfering RNA (siRNA) and DNA molecules and ribozymes that function to inhibit translation of a polypeptide. Antisense techniques and RNA interference techniques are known in the art and are described herein.

Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. For example, hammerhead motif ribozyme molecules may be engineered that specifically and efficiently catalyze endonucleolytic cleavage of RNA sequences corresponding to or complementary to the nucleotide sequences set forth in FIGS. 1A-1S or FIG. 2. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including breast cancer, resulting from aberrant expression of a target gene described herein. For example, the nucleic acid sequence may be used in hybridization assays of biopsies or autopsies to diagnose abnormalities of expression or function (e.g., Southern or Northern blot analysis, in situ hybridization assays).

In addition, the expression of a polypeptide during embryonic development may also be determined using nucleic acid encoding the polypeptide. As addressed infra, production of functionally impaired polypeptide is the cause of various disease states, breast cancer. In situ hybridizations using polypeptide as a probe may be employed to predict problems related to breast cancer. Further, as indicated infra, administration of human active polypeptide, recombinantly produced as described herein, may be used to treat disease states related to functionally impaired polypeptide. Alternatively, gene therapy approaches may be employed to remedy deficiencies of functional polypeptide or to replace or compete with dysfunctional polypeptide.

Expression Vectors Host Cells and Genetically Engineered Cells

Provided herein are nucleic acid vectors, often expression vectors, which contain a nucleotide sequence set forth in FIGS. 1A-1S or FIG. 2 or a substantially identical sequence thereof. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include a nucleotide sequence from FIGS. 1A-1S or FIG. 2 in a form suitable for expression of an encoded RAD21 polypeptide or RAD21 nucleic acid in a host cell. A “RAD21 polypeptide” is a polypeptide encoded by a nucleotide sequence from FIGS. 1A-1S or FIG. 2 or a substantially identical nucleotide sequence thereof. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce RAD21 polypeptides, including fusion polypeptides.

Recombinant expression vectors can be designed for expression of RAD21 polypeptides in prokaryotic or eukaryotic cells. For example, RAD21 polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 31-40 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for RAD21 polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol. 43: 235-275 (1988)), promoters of T cell receptors (Winoto & Baltimore, EMBO J. 8: 729-733 (1989)) promoters of immunoglobulins (Banetji et al., Cell 33: 729-740 (1983); Queen & Baltimore, Cell 33: 741-748 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters are sometimes utilized, for example, the murine hox promoters (Kessel & Gruss, Science 249: 374-379 (1990)) and the α-fetopolypeptide promoter (Campes & Tilghman, Genes Dev. 3: 537-546 (1989)).

A nucleic acid from FIGS. 1A-1S or FIG. 2 may also be cloned into an expression vector in an antisense orientation. Regulatory sequences (e.g., viral promoters and/or enhancers) operatively linked to a nucleic acid of FIGS. 1A-1S or FIG. 2 cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see, e.g., Weintraub et al., Antisense RNA as a molecular tool for genetic analysis, Reviews—Trends in Genetics, Vol. 1(1) (1986).

Also provided herein are host cells that include a nucleotide sequence from FIGS. 1A-1S or FIG. 2 within a recombinant expression vector or a fragment of a nucleotide sequence from FIGS. 1A-1S or FIG. 2 which facilitate homologous recombination into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a RAD21 polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a RAD21 polypeptide. Accordingly, further provided are methods for producing a RAD21 polypeptide using the host cells. In one embodiment, the method includes culturing host cells into which a recombinant expression vector encoding a RAD21 polypeptide has been introduced in a suitable medium such that a RAD21 polypeptide is produced. In another embodiment, the method further includes isolating a RAD21 polypeptide from the medium or the host cell.

Also provided are cells or purified preparations of cells which include a transgene from FIGS. 1A-1S or FIG. 2, or which otherwise misexpress RAD21 polypeptide. Cell preparations can consist of human or non-human cells, e.g., rodent cells, e.g., mouse or rat cells, rabbit cells, or pig cells. In certain embodiments, the cell or cells include a transgene from FIGS. 1A-1S or FIG. 2 (e.g., a heterologous form of a gene in FIGS. 1A-1S or FIG. 2, such as a human gene expressed in non-human cells). The transgene can be misexpressed, e.g., overexpressed or underexpressed. In other embodiments, the cell or cells include a gene which misexpress an endogenous RAD21 polypeptide (e.g., expression of a gene is disrupted, also known as a knockout). Such cells can serve as a model for studying disorders which are related to mutated or mis-expressed alleles or for use in drug screening. Also provided are human cells (e.g., a hematopoietic stem cells) transformed with a nucleic acid from FIGS. 1A-1S or FIG. 2.

Also provided are cells or a purified preparation thereof (e.g., human cells) in which an endogenous nucleic acid from FIGS. 1A-1S or FIG. 2 is under the control of a regulatory sequence that does not normally control the expression of the endogenous gene corresponding to the sequence from FIGS. 1A-1S or FIG. 2. The expression characteristics of an endogenous gene within a cell (e.g., a cell line or microorganism) can be modified by inserting a heterologous DNA regulatory element into the genome of the cell such that the inserted regulatory element is operably linked to the corresponding endogenous gene. For example, an endogenous corresponding gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

Transgenic Animals

Non-human transgenic animals that express a heterologous RAD21 polypeptide (e.g., expressed from a nucleic acid from FIGS. 1A-1S or FIG. 2 or substantially identical sequence thereof) can be generated. Such animals are useful for studying the function and/or activity of a RAD21 polypeptide and for identifying and/or evaluating modulators of the activity of nucleic acids from FIGS. 1A-1S or FIG. 2 and encoded polypeptides. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat), a bird (e.g., a chicken or a turkey), an amphibian (e.g., a frog, salamander, or newt), or an insect (e.g., Drosophila melanogaster), in which one or more of the cells of the animal includes a transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous nucleic acid homologous to a nucleic acid from FIGS. 1A-1S or FIG. 2 has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to a nucleotide sequence of FIGS. 1A-1S or FIG. 2 to direct expression of an encoded polypeptide to particular cells. A transgenic founder animal can be identified based upon the presence of a nucleotide sequence from FIGS. 1A-1S or FIG. 2 in its genome and/or expression of encoded mRNA in tissues or cells of the animals. A transgenic founder animal can then be used to breed additional animals carrying the transgene. Moreover, transgenic animals carrying a nucleotide sequence from FIGS. 1A-1S or FIG. 2 can further be bred to other transgenic animals carrying other transgenes.

RAD21 polypeptides can be expressed in transgenic animals or plants by introducing, for example, a nucleic acid from FIGS. 1A-1S or FIG. 2 into the genome of an animal that encodes the RAD21 polypeptide. In certain embodiments the nucleic acid is placed under the control of a tissue specific promoter, e.g., a milk or egg specific promoter, and recovered from the milk or eggs produced by the animal. Also included is a population of cells from a transgenic animal.

RAD21 Polypeptides

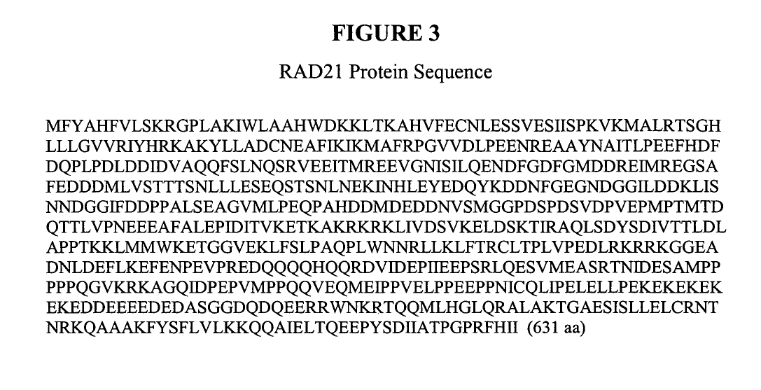

Also featured herein are isolated RAD21 polypeptides, which are encoded by a nucleotide sequence from FIGS. 1A-1S or FIG. 2 or a substantially identical nucleotide sequence thereof, or alternatively are set forth in FIG. 3. Isolated RAD21 polypeptides featured herein include both the full-length polypeptide and the mature polypeptide (i.e., the polypeptide minus the signal sequence or propeptide domain). An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a RAD21 polypeptide having less than about 30%, 20%, 10% and more preferably 5% (by dry weight), of non-RAD21 polypeptide (also referred to herein as a “contaminating protein”), or of chemical precursors or non-target chemicals. When the RAD21 polypeptide or a biologically active portion thereof is recombinantly produced, it is also preferably substantially free of culture medium, specifically, where culture medium represents less than about 20%, sometimes less than about 10%, and often less than about 5% of the volume of the polypeptide preparation. Isolated or purified RAD21 polypeptide preparations are sometimes 0.01 milligrams or more or 0.1 milligrams or more, and often 1.0 milligrams or more and 10 milligrams or more in dry weight.

Further included herein are RAD21 polypeptide fragments. The polypeptide fragment may be a domain or part of a domain of a RAD21 polypeptide. RAD21 domains include, but are not limited to, transmembrane domains (142-165, 195-212, 286-309, 344-361 amino acids of FIG. 3), the protease domain (278-455 amino acids of FIG. 3), and the Zn-binding motif (HEXXH) present in the protease domain (327-331 amino acids of FIG. 3) The polypeptide fragment may have increased, decreased or unexpected biological activity. The polypeptide fragment is often 50 or fewer, 100 or fewer, or 200 or fewer amino acids in length, and is sometimes 300, 400, 500, 524 or fewer amino acids in length.

Substantially identical RAD21 polypeptides may depart from the amino acid sequences of RAD21 polypeptides in different manners. For example, conservative amino acid modifications may be introduced at one or more positions in the amino acid sequences of RAD21 polypeptides. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a RAD21 polypeptide, whereas altering an “essential” amino acid abolishes or substantially alters the biological function of a RAD21 polypeptide. Amino acids that are conserved among RAD21 polypeptides are typically essential amino acids.

Also, RAD21 polypeptides may exist as chimeric or fusion polypeptides. As used herein, a target “chimeric polypeptide” or target “fusion polypeptide” includes a RAD21 polypeptide linked to a non-RAD21 polypeptide. A “non-RAD21 polypeptide” refers to a polypeptide having an amino acid sequence corresponding to a polypeptide which is not substantially identical to the RAD21 polypeptide, which includes, for example, a polypeptide that is different from the RAD21 polypeptide and derived from the same or a different organism. The RAD21 polypeptide in the fusion polypeptide can correspond to an entire or nearly entire RAD21 polypeptide or a fragment thereof. The non-RAD21 polypeptide can be fused to the N-terminus or C-terminus of the RAD21 polypeptide.

Fusion polypeptides can include a moiety having high affinity for a ligand. For example, the fusion polypeptide can be a GST-target fusion polypeptide in which the target sequences are fused to the C-terminus of the GST sequences, or a polyhistidine-target fusion polypeptide in which the RAD21 polypeptide is fused at the N- or C-terminus to a string of histidine residues. Such fusion polypeptides can facilitate purification of recombinant RAD21 polypeptide. Expression vectors are commercially available that already encode a fusion moiety (e.g., a GST polypeptide), and a nucleotide sequence from FIGS. 1A-1S or FIG. 2, or a substantially identical nucleotide sequence thereof, can be cloned into an expression vector such that the fusion moiety is linked in-frame to the RAD21 polypeptide. Further, the fusion polypeptide can be a RAD21 polypeptide containing a heterologous signal sequence at its N-terminus. In certain host cells (e.g., mammalian host cells), expression, secretion, cellular internalization, and cellular localization of a RAD21 polypeptide can be increased through use of a heterologous signal sequence. Fusion polypeptides can also include all or a part of a serum polypeptide (e.g., an IgG constant region or human serum albumin).

RAD21 polypeptides can be incorporated into pharmaceutical compositions and administered to a subject in vivo. Administration of these RAD21 polypeptides can be used to affect the bioavailability of a substrate of the RAD21 polypeptide and may effectively increase RAD21 polypeptide biological activity in a cell. Target fusion polypeptides may be useful therapeutically for the treatment of disorders caused by, for example, (i) aberrant modification or mutation of a gene encoding a RAD21 polypeptide; (ii) mis-regulation of the gene encoding the RAD21 polypeptide; and (iii) aberrant post-translational modification of a RAD21 polypeptide. Also, RAD21 polypeptides can be used as immunogens to produce anti-target antibodies in a subject, to purify RAD21 polypeptide ligands or binding partners, and in screening assays to identify molecules which inhibit or enhance the interaction of a RAD21 polypeptide with a substrate.

In addition, polypeptides can be chemically synthesized using techniques known in the art (See, e.g., Creighton, 1983 Proteins. New York, N.Y.: W. H. Freeman and Company; and Hunkapiller et al., (1984) Nature July 12-18;310(5973):105-11). For example, a relative short fragment can be synthesized by use of a peptide synthesizer. Furthermore, if desired, non-classical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, omithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Polypeptides and polypeptide fragments sometimes are differentially modified during or after translation, e.g., by glycosylation, acetylation, phosphorylation, amidation, derivatization by known protecting/blocking groups, proteolytic cleavage, linkage to an antibody molecule or other cellular ligand, and the like. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; and the like. Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptide fragments may also be modified with a detectable label, such as an enzymatic, fluorescent, isotopic or affinity label to allow for detection and isolation of the polypeptide.

Also provided are chemically modified derivatives of polypeptides that can provide additional advantages such as increased solubility, stability and circulating time of the polypeptide, or decreased immunogenicity (see e.g., U.S. Pat. No. 4,179,337. The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the molecular weight often is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polymers should be attached to the polypeptide with consideration of effects on functional or antigenic domains of the polypeptide. There are a number of attachment methods available to those skilled in the art (e.g., EP 0 401 384 (coupling PEG to G-CSF) and Malik et al. (1992) Exp Hematol. September;20(8):1028-35 (pegylation of GM-CSF using tresyl chloride)). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. For therapeutic purposes, the attachment sometimes is at an amino group, such as attachment at the N-terminus or lysine group.

Proteins can be chemically modified at the N-terminus. Using polyethylene glycol as an illustration of such a composition, one may select from a variety of polyethylene glycol molecules (by molecular weight, branching, and the like), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical Nucleic Acids and Polypeptides

Nucleotide sequences and polypeptide sequences that are substantially identical to the nucleotide sequences in FIGS. 1A-1S or FIG. 2 and the RAD21 polypeptide sequences encoded by those nucleotide sequences, respectively, are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55% or more, 60% or more, 65% or more, 70% or more, 75% or more, 80% or more, 85% or more, 90% or more, 95% or more (each often within a 1%, 2%, 3% or 4% variability) identical to the nucleotide sequences in FIGS. 1A-1S or FIG. 2 or the encoded RAD21 polypeptide amino acid sequences. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, 95% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

An example of a substantially identical nucleotide sequence to a nucleotide sequence in FIGS. 1A-1S or FIG. 2 is one that has a different nucleotide sequence but still encodes the same polypeptide sequence encoded by the nucleotide sequence in FIGS. 1A-1S or FIG. 2. Another example is a nucleotide sequence that encodes a polypeptide having a polypeptide sequence that is more than 70% or more identical to, sometimes more than 75% or more, 80% or more, or 85% or more identical to, and often more than 90% or more and 95% or more identical to a polypeptide sequence encoded by a nucleotide sequence in FIGS. 1A-1S or FIG. 2.

Nucleotide sequences from FIGS. 1A-1S or FIG. 2 and amino acid sequences of encoded polypeptides can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences from FIGS. 1A-1S or FIG. 2. BLAST polypeptide searches can be performed with the XBLAST program, score=50, wordlength=3 to obtain amino acid sequences homologous to polypeptides encoded by the nucleotide sequences of FIGS. 1A-1S or FIG. 2. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

A nucleic acid that is substantially identical to a nucleotide sequence in FIGS. 1A-1S or FIG. 2 may include polymorphic sites at positions equivalent to those described herein when the sequences are aligned. For example, using the alignment procedures described herein, SNPs in a sequence substantially identical to a sequence in FIGS. 1A-1S or FIG. 2 can be identified at nucleotide positions that match (i.e., align) with nucleotides at SNP positions in each nucleotide sequence in FIGS. 1A-1S or FIG. 2. Also, where a polymorphic variation results in an insertion or deletion, insertion or deletion of a nucleotide sequence from a reference sequence can change the relative positions of other polymorphic sites in the nucleotide sequence.

Substantially identical nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50%, about 55% or more, often about 70-75% or more, more often about 80-85% or more, and typically about 90-95% or more identical to the amino acid sequences of RAD21 polypeptides or a fragment thereof. Such nucleic acid molecules can readily be identified as being able to hybridize under stringent conditions to a nucleotide sequence in FIGS. 1A-1S or FIG. 2 or a fragment of this sequence. Nucleic acid molecules corresponding to orthologs, homologs, and allelic variants of a nucleotide sequence in FIGS. 1A-1S or FIG. 2 can further be identified by mapping the sequence to the same chromosome or locus as the nucleotide sequence in FIGS. 1A-1S or FIG. 2.

Also, substantially identical nucleotide sequences may include codons that are altered with respect to the naturally occurring sequence for enhancing expression of a RAD21 polypeptide in a particular expression system. For example, the nucleic acid can be one in which one or more codons are altered, and often 10% or more or 20% or more of the codons are altered for optimized expression in bacteria (e.g., E. coli.), yeast (e.g., S. cervesiae), human (e.g., 293 cells), insect, or rodent (e.g., hamster) cells.

Methods for Identifying Subjects at Risk of Breast Cancer and Risk of Breast Cancer in a Subject

Methods for determining whether a subject is at risk of breast cancer are provided herein. These methods include detecting the presence or absence of one or more polymorphic variations associated with breast cancer in a RAD21 nucleotide sequence, or substantially identical sequence thereof, in a sample from a subject, where the presence of such a polymorphic variation is indicative of the subject being at risk of breast cancer. These genetic tests are useful for prognosing and/or diagnosing breast cancer and often are useful for determining whether an individual is at an increased, intermediate or decreased risk of developing or having breast cancer.

Thus, featured herein is a method for identifying a subject at risk of breast cancer, which comprises detecting in a nucleic acid sample from the subject the presence or absence of a polymorphic variation associated with breast cancer at a polymorphic site in a RAD21 nucleotide sequence. The nucleotide sequence often is selected from the group consisting of: (a) a nucleotide sequence set forth in FIGS. 1A-1S or FIG. 2; (b) a nucleotide sequence which encodes a polypeptide consisting of an amino acid sequence encoded by a nucleotide sequence set forth in FIGS. 1A-1S or FIG. 2; (c) a nucleotide sequence which encodes a polypeptide that is 90% or more identical to an amino acid sequence encoded by a nucleotide sequence set forth in FIGS. 1A-1S or FIG. 2 or a nucleotide sequence about 90% or more identical to the nucleotide sequence set forth in FIGS. 1A-1S or FIG. 2; and (d) a fragment of a nucleotide sequence of (a), (b), or (c), where the fragment comprises a polymorphic site; whereby the presence of the polymorphic variation is indicative of the subject being at risk of breast cancer. A polymorphic variation assayed in the genetic test often is located in an intron, sometimes in a region surrounding the RAD21 open reading frame (e.g., within 50 kilobases (kb), 40 kb, 30 kb, 20 kb, 15, kb, 10 kb, 5 kb, 4 kb, 3 kb, 2 kb, or 1 kb of the open reading frame initiation site or termination site), and sometimes in an exon. Sometimes the polymorphic variation is not located in an exon. In an embodiment, a polymorphic variant at NCBI SNP ID rs1374297 is detected for determining a risk of breast cancer.

A risk of developing aggressive forms of breast cancer likely to metastasize or invade surrounding tissues (e.g., Stage IIIA, IIIB, and IV breast cancers), and subjects at risk of developing aggressive forms of breast cancer also may be identified by the methods described herein. These methods include collecting phenotype information from subjects having breast cancer, which includes the stage of progression of the breast cancer, and performing a secondary phenotype analysis to detect the presence or absence of one or more polymorphic variations associated with a particular stage form of breast cancer. Thus, detecting the presence or absence of one or more polymorphic variations in a RAD21 nucleotide sequence associated with a late stage form of breast cancer often is diagnostic of an aggressive form of the cancer.

Results from prognostic tests may be combined with other test results to diagnose breast cancer. For example, prognostic results may be gathered, a patient sample may be ordered based on a determined predisposition to breast cancer, the patient sample is analyzed, and the results of the analysis may be utilized to diagnose breast cancer. Also breast cancer diagnostic methods can be developed from studies used to generate prognostic/diagnostic methods in which populations are stratified into subpopulations having different progressions of breast cancer. In another embodiment, prognostic results may be gathered; a patient's risk factors for developing breast cancer analyzed (e.g., age, race, family history, age of first menstrual cycle, age at birth of first child); and a patient sample may be ordered based on a determined predisposition to breast cancer. In an alternative embodiment, the results from predisposition analyses described herein may be combined with other test results indicative of breast cancer, which were previously, concurrently, or subsequently gathered with respect to the predisposition testing. In these embodiments, the combination of the prognostic test results with other test results can be probative of breast cancer, and the combination can be utilized as a breast cancer diagnostic. The results of any test indicative of breast cancer known in the art may be combined with the methods described herein. Examples of such tests are mammography (e.g., a more frequent and/or earlier mammography regimen may be prescribed); breast biopsy and optionally a biopsy from another tissue; breast ultrasound and optionally an ultrasound analysis of another tissue; breast magnetic resonance imaging (MRI) and optionally an MRI analysis of another tissue; electrical impedance (T-scan) analysis of breast and optionally of another tissue; ductal lavage; nuclear medicine analysis (e.g., scintimammography); BRCA1 and/or BRCA2 sequence analysis results; and thermal imaging of the breast and optionally of another tissue. Testing may be performed on tissue other than breast to diagnose the occurrence of metastasis (e.g., testing of the lymph node).

Risk of breast cancer sometimes is expressed as a probability, such as an odds ratio, percentage, or risk factor. The risk is based upon the presence or absence of one or more polymorphic variants described herein, and also may be based in part upon phenotypic traits of the individual being tested. Methods for calculating risk based upon patient data are well known (see, e.g., Agresti, Categorical Data Analysis, 2nd Ed. 2002. Wiley). Allelotyping and genotyping analyses may be carried out in populations other than those exemplified herein to enhance the predictive power of the prognostic method. These further analyses are executed in view of the exemplified procedures described herein, and may be based upon the same polymorphic variations or additional polymorphic variations. Risk determinations for breast cancer are useful in a variety of applications. In one embodiment, breast cancer risk determinations are used by clinicians to direct appropriate detection, preventative and treatment procedures to subjects who most require these. In another embodiment, breast cancer risk determinations are used by health insurers for preparing actuarial tables and for calculating insurance premiums.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g., U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon a nucleotide sequence set forth in SEQ ID NO: 1 or 2 without undue experimentation using knowledge readily available in the art.

Also provided is an extension oligonucleotide that hybridizes to the amplified fragment adjacent to the polymorphic variation. As used herein, the term “adjacent” refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,127; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2. Multiple extension oligonucleotides may be utilized in one reaction, which is referred to herein as “multiplexing.”

A microarray can be utilized for determining whether a polymorphic variant is present or absent in a nucleic acid sample. A microarray may include any oligonucleotides described herein, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic site set forth in FIG. 1 or below.

A kit also may be utilized for determining whether a polymorphic variant is present or absent in a nucleic acid sample. A kit often comprises one or more pairs of oligonucleotide primers useful for amplifying a fragment of a RAD21 nucleotide sequence or a substantially identical sequence thereof, where the fragment includes a polymorphic site. The kit sometimes comprises a polymerizing agent, for example, a thermostable nucleic acid polymerase such as one disclosed in U.S. Pat. Nos. 4,889,818 or 6,077,664. Also, the kit often comprises an elongation oligonucleotide that hybridizes to a RAD21 nucleotide sequence in a nucleic acid sample adjacent to the polymorphic site. Where the kit includes an elongation oligonucleotide, it also often comprises chain elongating nucleotides, such as DATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. In an embodiment, the kit comprises one or more oligonucleotide primer pairs, a polymerizing agent, chain elongating nucleotides, at least one elongation oligonucleotide, and one or more chain terminating nucleotides. Kits optionally include buffers, vials, microtiter plates, and instructions for use.

An individual identified as being at risk of breast cancer may be heterozygous or homozygous with respect to the allele associated with a higher risk of breast cancer. A subject homozygous for an allele associated with an increased risk of breast cancer is at a comparatively high risk of breast cancer, a subject heterozygous for an allele associated with an increased risk of breast cancer is at a comparatively intermediate risk of breast cancer, and a subject homozygous for an allele associated with a decreased risk of breast cancer is at a comparatively low risk of breast cancer. A genotype may be assessed for a complementary strand, such that the complementary nucleotide at a particular position is detected.

Also featured are methods for determining risk of breast cancer and/or identifying a subject at risk of breast cancer by contacting a polypeptide or protein encoded by a RAD21 nucleotide sequence from a subject with an antibody that specifically binds to an epitope associated with increased risk of breast cancer in the polypeptide.

Applications of Prognostic and Diagnostic Results to Pharmacogenomic Methods

Pharmacogenomics is a discipline that involves tailoring a treatment for a subject according to the subject's genotype. For example, based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects). As therapeutic approaches for breast cancer continue to evolve and improve, the goal of treatments for breast cancer related disorders is to intervene even before clinical signs (e.g., identification of lump in the breast) first manifest. Thus, genetic markers associated with susceptibility to breast cancer prove useful for early diagnosis, prevention and treatment of breast cancer.

The following is an example of a pharmacogenomic embodiment. A particular treatment regimen can exert a differential effect depending upon the subject's genotype. Where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The methods described herein are applicable to pharmacogenomic methods for detecting, preventing, alleviating and/or treating breast cancer. For example, a nucleic acid sample from an individual may be subjected to a genetic test described herein. Where one or more polymorphic variations associated with increased risk of breast cancer are identified in a subject, information for detecting, preventing or treating breast cancer and/or one or more breast cancer detection, prevention and/or treatment regimens then may be directed to and/or prescribed to that subject.

In certain embodiments, a detection, prevenative and/or treatment regimen is specifically prescribed and/or administered to individuals who will most benefit from it based upon their risk of developing breast cancer assessed by the methods described herein. Thus, provided are methods for identifying a subject at risk of breast cancer and then prescribing a detection, therapeutic or preventative regimen to individuals identified as being at risk of breast cancer. Thus, certain embodiments are directed to methods for treating breast cancer in a subject, reducing risk of breast cancer in a subject, or early detection of breast cancer in a subject, which comprise: detecting the presence or absence of a polymorphic variant associated with breast cancer in a nucleotide sequence set forth in SEQ ID NO: 1 or 2 in a nucleic acid sample from a subject, where the nucleotide sequence comprises a polynucleotide sequence selected from the group consisting of: (a) a nucleotide sequence set forth in SEQ ID NO: 1 or 2; (b) a nucleotide sequence which encodes a polypeptide having an amino acid sequence encoded by a nucleotide sequence in SEQ ID NO: 1 or 2; (c) a nucleotide sequence which encodes a polypeptide that is 90% or more identical to an amino acid sequence encoded by a nucleotide sequence in SEQ ID NO: 1 or 2 or a nucleotide sequence about 90% or more identical to the nucleotide sequence set forth in SEQ ID NO: 1 or 2; and (d) a fragment of a nucleotide sequence of (a), (b), or (c), sometimes comprising a polymorphic site associated with breast cancer; and prescribing or administering a breast cancer treatment regimen, preventative regimen and/or detection regimen to a subject from whom the sample originated where the presence of one or more polymorphic variations associated with breast cancer are detected in the nucleotide sequence. In these methods, genetic results may be utilized in combination with other test results to diagnose breast cancer as described above. Other test results include but are not limited to mammography results, imaging results, biopsy results and results from BRCA1 or BRAC2 test results, as described above.

Detection regimens include one or more mammography procedures, a regular mammography regimen (e.g., once a year, or once every six, four, three or two months); an early mammography regimen (e.g., mammography tests are performed beginning at age 25, 30, or 35); one or more biopsy procedures (e.g., a regular biopsy regimen beginning at age 40); breast biopsy and biopsy from other tissue; breast ultrasound and optionally ultrasound analysis of another tissue; breast magnetic resonance imaging (MRI) and optionally MRI analysis of another tissue; electrical impedance (T-scan) analysis of breast and optionally another tissue; ductal lavage; nuclear medicine analysis (e.g., scintimammography); BRCA1 and/or BRCA2 sequence analysis results; and/or thermal imaging of the breast and optionally another tissue.

Treatments sometimes are preventative (e.g., is prescribed or administered to reduce the probability that a breast cancer associated condition arises or progresses), sometimes are therapeutic, and sometimes delay, alleviate or halt the progression of breast cancer. Any known preventative or therapeutic treatment for alleviating or preventing the occurrence of breast cancer is prescribed and/or administered. For example, certain preventative treatments often are prescribed to subjects having a predisposition to breast cancer and where the subject is not diagnosed with breast cancer or is diagnosed as having symptoms indicative of early stage breast cancer (e.g., stage I). For subjects not diagnosed as having breast cancer, any preventative treatments known in the art can be prescribed and administered, which include selective hormone receptor modulators (e.g., selective estrogen receptor modulators (SERMs) such as tamoxifen, reloxifene, and toremifene); compositions that prevent production of hormones (e.g., aramotase inhibitors that prevent the production of estrogen in the adrenal gland, such as exemestane, letrozole, anastrozol, groserelin, and megestrol); other hormonal treatments (e.g., goserelin acetate and fulvestrant); biologic response modifiers such as antibodies (e.g., trastuzumab (herceptin/HER2)); surgery (e.g., lumpectomy and mastectomy); drugs that delay or halt metastasis (e.g., pamidronate disodium); and alternative/complementary medicine (e.g., acupuncture, acupressure, moxibustion, qi gong, reiki, ayurveda, vitamins, minerals, and herbs (e.g., astragalus root, burdock root, garlic, green tea, and licorice root)).

The use of breast cancer treatments are well known in the art, and include surgery, chemotherapy and/or radiation therapy. Any of the treatments may be used in combination to treat or prevent breast cancer (e.g., surgery followed by radiation therapy or chemotherapy). Examples of chemotherapy combinations used to treat breast cancer include: cyclophosphamide (Cytoxan), methotrexate (Amethopterin, Mexate, Folex), and fluorouracil (Fluorouracil, 5-Fu, Adrucil), which is referred to as CMF; cyclophosphamide, doxorubicin (Adriamycin), and fluorouracil, which is referred to as CAF; and doxorubicin (Adriamycin) and cyclophosphamide, which is referred to as AC.

As breast cancer preventative and treatment information can be specifically targeted to subjects in need thereof (e.g., those at risk of developing breast cancer or those that have early signs of breast cancer), provided herein is a method for preventing or reducing the risk of developing breast cancer in a subject, which comprises: (a) detecting the presence or absence of a polymorphic variation associated with breast cancer at a polymorphic site in a nucleotide sequence in a nucleic acid sample from a subject; (b) identifying a subject at risk of breast cancer, whereby the presence of the polymorphic variation is indicative of a risk of breast cancer in the subject; and (c) if such a risk is identified, providing the subject with information about methods or products to prevent or reduce breast cancer or to delay the onset of breast cancer. Also provided is a method of targeting information or advertising to a subpopulation of a human population based on the subpopulation being genetically predisposed to a disease or condition, which comprises: (a) detecting the presence or absence of a polymorphic variation associated with breast cancer at a polymorphic site in a nucleotide sequence in a nucleic acid sample from a subject; (b) identifying the subpopulation of subjects in which the polymorphic variation is associated with breast cancer; and (c) providing information only to the subpopulation of subjects about a particular product which may be obtained and consumed or applied by the subject to help prevent or delay onset of the disease or condition.

Pharmacogenomics methods also may be used to analyze and predict a response to a breast cancer treatment or a drug. For example, if pharmacogenomics analysis indicates a likelihood that an individual will respond positively to a breast cancer treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The methods described herein also are applicable to clinical drug trials. One or more polymorphic variants indicative of response to an agent for treating breast cancer or to side effects to an agent for treating breast cancer may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems. In certain embodiments, the agent for treating breast cancer described herein targets RAD21 or a target in the RAD21 pathway (e.g., Rho GTPase).

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (c) including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a polynucleotide sequence set forth in SEQ ID NO: 1 or 2; (ii) a polynucleotide sequence that is 90% or more identical to a nucleotide sequence set forth in SEQ I[) NO: 1 or 2; (iii) a polynucleotide sequence that encodes a polypeptide having an amino acid sequence identical to or 90% or more identical to an amino acid sequence encoded by a nucleotide sequence set forth in SEQ ID NO: 1 or 2; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with breast cancer at a polymorphic site in a nucleotide sequence in a nucleic acid sample from a subject; (b) the diagnostic/prognostic testing provider identifies the subpopulation of subjects in which the polymorphic variation is associated with breast cancer; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising Breast Cancer-Directed Molecules

Featured herein is a composition comprising a breast cancer cell and one or more molecules specifically directed and targeted to a nucleic acid comprising a RAD21 nucleotide sequence or a RAD21 polypeptide. Such directed molecules include, but are not limited to, a compound that binds to a RAD21 nucleic acid or a RAD21 polypeptide; a RNAi or siRNA molecule having a strand complementary to a RAD21 nucleotide sequence; an antisense nucleic acid complementary to an RNA encoded by a RAD21 DNA sequence; a ribozyme that hybridizes to a RAD21 nucleotide sequence; a nucleic acid aptamer that specifically binds a RAD21 polypeptide; and an antibody that specifically binds to a RAD21 polypeptide or binds to a RAD21 nucleic acid. In specific embodiments, the breast cancer directed molecule interacts with a RAD21 nucleic acid or polypeptide variant associated with breast cancer. In other embodiments, the breast cancer directed molecule interacts with a polypeptide involved in the RAD21 signal pathway, or a nucleic acid encoding such a polypeptide.

Compositions sometimes include an adjuvant known to stimulate an immune response, and in certain-embodiments, an adjuvant that stimulates a T-cell lymphocyte response. Adjuvants are known, including but not limited to an aluminum adjuvant (e.g., aluminum hydroxide); a cytokine adjuvant or adjuvant that stimulates a cytokine response (e.g., interleukin (IL)-12 and/or γ-interferon cytokines); a Freund-type mineral oil adjuvant emulsion (e.g., Freund's complete or incomplete adjuvant); a synthetic lipoid compound; a copolymer adjuvant (e.g., TitreMax); a saponin; Quil A; a liposome; an oil-in-water emulsion (e.g., an emulsion stabilized by Tween 80 and pluronic polyoxyethlene/polyoxypropylene block copolymer (Syntex Adjuvant Formulation); TitreMax; detoxified endotoxin (MPL) and mycobacterial cell wall components (TDW, CWS) in 2% squalene (Ribi Adjuvant System)); a muramyl dipeptide; an immune-stimulating complex (ISCOM, e.g., an Ag-modified saponin/cholesterol micelle that forms stable cage-like structure); an aqueous phase adjuvant that does not have a depot effect (e.g., Gerbu adjuvant); a carbohydrate polymer (e.g., AdjuPrime); L-tyrosine; a manide-oleate compound (e.g., Montanide); an ethylene-vinyl acetate copolymer (e.g., Elvax 40W1,2); or lipid A, for example. Such compositions are useful for generating an immune response against a breast cancer directed molecule (e.g., an HLA-binding subsequence within a polypeptide encoded by a nucleotide sequence in SEQ ID NO: 1). In such methods, a peptide having an amino acid subsequence of a polypeptide encoded by a nucleotide sequence in SEQ ID NO: 1 or 2 is delivered to a subject, where the subsequence binds to an HLA molecule and induces a CTL lymphocyte response. The peptide sometimes is delivered to the subject as an isolated peptide or as a minigene in a plasmid that encodes the peptide. Methods for identifying HLA-binding subsequences in such polypeptides are known (see e.g., publication WO02/20616 and PCT application US98/01373 for methods of identifying such sequences).

The breast cancer cell may be in a group of breast cancer cells and/or other types of cells cultured in vitro or in a tissue having breast cancer cells (e.g., a melanocytic lesion) maintained in vitro or present in an animal in vivo (e.g., a rat, mouse, ape or human). In certain embodiments, a composition comprises a component from a breast cancer cell or from a subject having a breast cancer cell instead of the breast cancer cell or in addition to the breast cancer cell, where the component sometimes is a nucleic acid molecule (e.g., genomic DNA), a protein mixture or isolated protein, for example. The aforementioned compositions have utility in diagnostic, prognostic and pharmacogenomic methods described previously and in breast cancer therapeutics described hereafter. Certain breast cancer molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem.37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or on phage (Scott and Smith, Science 249: 386-390 (1990); Devlin, Science 249: 404406 (1990); Cwirla et al., Proc. Natl. Acad. Sci. 87: 6378-6382 (1990); Felici, J. Mol. Biol. 222: 301-310 (1991); Ladner supra.).

A compound sometimes alters expression and sometimes alters activity of a RAD21 polypeptide and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Antisense Nucleic Acid Molecules, Ribozymes, RNAi, siRNA and Modified Nucleic Acid Molecules

An “antisense” nucleic acid refers to a nucleotide sequence complementary to a “sense” nucleic acid encoding a polypeptide, e.g., complementary to the, coding strand of a double-stranded cDNA molecule or complementary to an mRNA sequence. The antisense nucleic acid can be complementary to an entire coding strand in SEQ ID NO: 1 or 2, or to a portion thereof or a substantially identical sequence thereof. In another embodiment, the antisense nucleic acid molecule is antisense to a “noncoding region” of the coding strand of a nucleotide sequence in SEQ ID NO: I (e.g., 5′ and 3′ untranslated regions).

An antisense nucleic acid can be designed such that it is complementary to the entire coding region of an mRNA encoded by a nucleotide sequence in SEQ ID NO: 1 (e.g., SEQ ID NO: 2), and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target a nucleotide sequence in SEQ ID NO: 1 or 2, often a variant associated with breast cancer, or a substantially identical sequence thereof. Among the variants, minor alleles and major alleles can be targeted, and those associated with a higher risk of breast cancer are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are *-anomeric nucleic acid molecules. An *-anomeric nucleic acid molecule forms specific double-stranded hybrids with complementary RNA in which, contrary to the usual *-units, the strands run parallel to each other (Gaultier et al., Nucleic Acids. Res. 15: 6625-6641 (1987)). Antisense nucleic acid molecules can also comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for a RAD21 nucleotide sequence can include one or more sequences complementary to such a nucleotide sequence, and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (see e.g., Cech et al. U.S. Pat. No. 4,987,071; and Cech et al. U.S. Pat. No. 5,116,742). Also, target mRNA sequences can be used to select a catalytic RNA having a specific ribonuclease activity from a pool of RNA molecules (see e.g., Bartel & Szostak, Science 261: 1411-1418 (1993)).

Breast cancer directed molecules include in certain embodiments nucleic acids that can form triple helix structures with a RAD21 nucleotide sequence or a substantially identical sequence thereof, especially one that includes a regulatory region that controls expression of a polypeptide. Gene expression can be inhibited by targeting nucleotide sequences complementary to the regulatory region of a RAD21 nucleotide sequence or a substantially identical sequence (e.g., promoter and/or enhancers) to form triple helical structures that prevent transcription of a gene in target cells (see e.g., Helene, Anticancer Drug Des. 6(6): 569-84 (1991); Helene et al., Ann. N.Y. Acad. Sci. 660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Breast cancer directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al., U.S. Pat. No. 6,506,559; Tuschl et al. PCT International Publication No. WO 01/75164; Kay et al. PCT International Publication No. WO 03/010180A1; or Bosher J M, Labouesse, Nat Cell Biol Febrary 2000;2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci USA. Aug. 14, 2001;98(17):9742-7 and Elbashir et al. Methods February 2000;26(2):199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. patent application No. US2001000993183; Caplen et al. Proc Natl Acad Sci USA; and Abderrahmani et al. Mol Cell Biol Nov. 21, 2001(21):7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA.

When designing the siRNA molecules, the targeted region often is selected from a given DNA sequence beginning 50 to 100 nucleotides downstream of the start codon. See, e.g., Elbashir et al,. Methods 26:199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (N, an nucleotide), and regions with approximately 30% to 70% G/C-content (often about 50% G/C-content) often are selected. If no suitable sequences are found, the search often is extended using the motif NA(N2 1). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N17)YNN, where R is purine (A,G) and Y is pyrimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, sometimes about 20-30 nucleotides in length or about 20-25 nucleotides in length, e.g., 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc.

Antisense, ribozyme, RNAi and siRNA nucleic acids can be altered to form modified nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 (1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNA nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNA nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization (Hyrup et al., (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. W088/09810) or the blood-brain barrier (see, e.g., PCT Publication No. W089/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988) ). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to a nucleotide sequence of SEQ ID NO: 1 or 2 or a substantially identical sequence thereof, two complementary regions one having a fluorophore and one a quencher such that the molecular beacon is useful for quantifying the presence of the nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)2 fragments which can be generated by treating the antibody with an enzyme such as pepsin. An antibody sometimes is a polyclonal, monoclonal, recombinant (e.g., a chimeric or humanized), fully human, non-human (e.g., murine), or a single chain antibody. An antibody may have effector function and can fix complement, and is sometimes coupled to a toxin or imaging agent.

A full-length polypeptide or antigenic peptide fragment encoded by a RAD21 nucleotide sequence can be used as an immunogen or can be used to identify antibodies made with other immunogens, e.g., cells, membrane preparations, and the like. An antigenic peptide often includes at least 8 amino acid residues of the amino acid sequences encoded by a nucleotide sequence of SEQ ID NO: 1, 2 or 3, or substantially identical sequence thereof, and encompasses an epitope. Antigenic peptides sometimes include 10 or more amino acids, 15 or more amino acids, 20 or more amino acids, or 30 or more amino acids. Hydrophilic and hydrophobic fragments of polypeptides sometimes are used as immunogens.

Epitopes encompassed by the antigenic peptide are regions located on the surface of the polypeptide (e.g., hydrophilic regions) as well as regions with high antigenicity. For example, an Emini surface probability analysis of the human polypeptide sequence can be used to indicate the regions that have a particularly high probability of being localized to the surface of the polypeptide and are thus likely to constitute surface residues useful for targeting antibody production. The antibody may bind an epitope on any domain or region on polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al European Patent Application 184,187; Taniguchi, M., European Patent Application 171,496; Morrison et al European Patent Application 173,494; Neuberger et al PCT International Publication No. WO 86/01533; Cabilly et al U.S. Pat. No. 4,816,567; Cabilly et al European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al., Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

An antibody can be a single chain antibody. A single chain antibody (scFV) can be engineered (see, e.g., Colcher et al., Ann. N Y Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1 dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An antibody (e.g., monoclonal antibody) can be used to isolate target polypeptides by standard techniques, such as affinity chromatography or immunoprecipitation. Moreover, an antibody can be used to detect a target polypeptide (e.g., in a cellular lysate or cell supernatant) in order to evaluate the abundance and pattern of expression of the polypeptide. Antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an antibody can be utilized as a test molecule for determining whether it can treat breast cancer, and as a therapeutic for administration to a subject for treating breast cancer.

An antibody can be made by immunizing with a purified antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native polypeptide, only denatured or otherwise non-native polypeptide, or which bind both, as well as those having linear or conformational epitopes. Conformational epitopes sometimes can be identified by selecting antibodies that bind to native but not denatured polypeptide. Also featured are antibodies that specifically bind to a polypeptide variant associated with breast cancer.

Screening Assays

Featured herein are methods for identifying a candidate therapeutic for treating breast cancer. The methods comprise contacting a test molecule with a target molecule in a system. A “target molecule” as used herein refers to a nucleic acid of SEQ ID NO: 1 or 2, a substantially identical nucleic acid thereof, or a fragment thereof, and an encoded polypeptide of the foregoing. The method also comprises determining the presence or absence of an interaction between the test molecule and the target molecule, where the presence of an interaction between the test molecule and the nucleic acid or polypeptide identifies the test molecule as a candidate breast cancer therapeutic. The interaction between the test molecule and the target molecule may be quantified.

Test molecules and candidate therapeutics include, but are not limited to, compounds, antisense nucleic acids, siRNA molecules, ribozymes, polypeptides or proteins encoded by a RAD21 nucleic acids, or a substantially identical sequence or fragment thereof, and immunotherapeutics (e.g., antibodies and HLA-presented polypeptide fragments). A test molecule or candidate therapeutic may act as a modulator of target molecule concentration or target molecule function in a system. A “modulator” may agonize (i.e., up-regulates) or antagonize (i.e., down-regulates) a target molecule concentration partially or completely in a system by affecting such cellular functions as DNA replication and/or DNA processing (e.g., DNA methylation or DNA repair), RNA transcription and/or RNA processing (e.g., removal of intronic sequences and/or translocation of spliced mRNA from the nucleus), polypeptide production (e.g., translation of the polypeptide from mRNA), and/or polypeptide post-translational modification (e.g., glycosylation, phosphorylation, and proteolysis of pro-polypeptides). A modulator may also agonize or antagonize a biological function of a target molecule partially or completely, where the function may include adopting a certain structural conformation, interacting with one or more binding partners, ligand binding, catalysis (e.g., phosphorylation, dephosphorylation, hydrolysis, methylation, and isomerization), and an effect upon a cellular event (e.g., effecting progression of breast cancer).

As used herein, the term “system” refers to a cell free in vitro environment and a cell-based environment such as a collection of cells, a tissue, an organ, or an organism. A system is “contacted” with a test molecule in a variety of manners, including adding molecules in solution and allowing them to interact with one another by diffusion, cell injection, and any administration routes in an animal. As used herein, the term “interaction” refers to an effect of a test molecule on test molecule, where the effect sometimes is binding between the test molecule and the target molecule, and sometimes is an observable change in cells, tissue, or organism.

There are many standard methods for detecting the presence or absence of interaction between a test molecule and a target molecule. For example, titrametric, acidimetric, radiometric, NMR, monolayer, polarographic, spectrophotometric, fluorescent, and ESR assays probative of a target molecule interaction may be utilized.

Test molecule/target molecule interactions can be detected and/or quantified using assays known in the art. For example, an interaction can be determined by labeling the test molecule and/or the target molecule, where the label is covalently or non-covalently attached to the test molecule or target molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. In addition, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and target molecule (McConnell, H. M. et al., Science 257: 1906-1912 (1992)).

In cell-based systems, cells typically include a nucleic acid from FIGS. 1A-1S or FIG. 2, an encoded polypeptide, or substantially identical nucleic acid or polypeptide thereof, and are often of mammalian origin, although the cell can be of any origin. Whole cells, cell homogenates, and cell fractions (e.g., cell membrane fractions) can be subjected to analysis. Where interactions between a test molecule with a RAD21 polypeptide are monitored, soluble and/or membrane bound forms of the polypeptide may be utilized. Where membrane-bound forms of the polypeptide are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl-N,N-dimethyl-3-ammonio-1-propane sulfonate.

An interaction between a test molecule and target molecule also can be detected by monitoring fluorescence energy transfer (FET) (see, e.g., Lakowicz et al., U.S. Pat. No. 5,631,169; Stavrianopoulos et al. U.S. Pat. No. 4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the presence or absence of an interaction between a test molecule and a target molecule can be effected by monitoring surface plasmon resonance (see, e.g., Sjolander & Urbaniczk, Anal. Chem. 63: 2338-2345 (1991) and Szabo et al., Curr. Opin. Struct. Biol. 5: 699-705 (1995)). “Surface plasmon resonance” or “biomolecular interaction analysis (BIA)” can be utilized to detect biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment, the target molecule or test molecules are anchored to a solid phase, facilitating the detection of target molecule/test molecule complexes and separation of the complexes from free, uncomplexed molecules. The target molecule or test molecule is immobilized to the solid support. In an embodiment, the target molecule is anchored to a solid surface, and the test molecule, which is not anchored, can be labeled, either directly or indirectly, with detectable labels discussed herein.

It may be desirable to immobilize a target molecule, an anti-target molecule antibody, and/or test molecules to facilitate separation of target molecule/test molecule complexes from uncomplexed forms, as well as to accommodate automation of the assay. The attachment between a test molecule and/or target molecule and the solid support may be covalent or non-covalent (see, e.g., U.S. Pat. No. 6,022,688 for non-covalent attachments). The solid support may be one or more surfaces of the system, such as one or more surfaces in each well of a microtiter plate, a surface of a silicon wafer, a surface of a bead (see, e.g., Lam, Nature 354: 82-84 (1991)) that is optionally linked to another solid support, or a channel in a microfluidic device, for example. Types of solid supports, linker molecules for covalent and non-covalent attachments to solid supports, and methods for immobilizing nucleic acids and other molecules to solid supports are well known (see, e.g., U.S. Pat. Nos. 6,261,776; 5,900,481; 6,133,436; and 6,022,688; and WIPO publication WO 01/18234).

In an embodiment, target molecule may be immobilized to surfaces via biotin and streptavidin. For example, biotinylated RAD21 polypeptide can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In another embodiment, a RAD21 polypeptide can be prepared as a fusion polypeptide. For example, glutathione-S-transferase/RAD21 polypeptide fusion can be adsorbed onto glutathione sepharose beads (Sigma Chemical, St. Louis, Mo.) or glutathione derivitized microtiter plates, which are then combined with a test molecule under conditions conducive to complex formation (e.g., at physiological conditions for salt and pH). Following incubation, the beads or microtiter plate wells are washed to remove any unbound components, or the matrix is immobilized in the case of beads, and complex formation is determined directly or indirectly as described above. Alternatively, the complexes can be dissociated from the matrix, and the level of target molecule binding or activity is determined using standard techniques.

In an embodiment, the non-immobilized component is added to the coated surface containing the anchored component. After the reaction is complete, unreacted components are removed (e.g., by washing) under conditions such that a significant percentage of complexes formed will remain immobilized to the solid surface. The detection of complexes anchored on the solid surface can be accomplished in a number of manners. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., by adding a labeled antibody specific for the immobilized component, where the antibody, in turn, can be directly labeled or indirectly labeled with, e.g., a labeled anti-Ig antibody.

In another embodiment, an assay is performed utilizing antibodies that specifically bind target molecule or test molecule but do not interfere with binding of the target molecule to the test molecule. Such antibodies can be derivitized to a solid support, and unbound target molecule may be immobilized by antibody conjugation. Methods for detecting such complexes, in addition to those described above for the GST-immobilized complexes, include immunodetection of complexes using antibodies reactive with the target molecule, as well as enzyme-linked assays which rely on detecting an enzymatic activity associated with the target molecule.

Cell free assays also can be conducted in a liquid phase. In such an assay, reaction products are separated from unreacted components, by any of a number of standard techniques, including but not limited to: differential centrifugation (see, e.g., Rivas, G., and Minton, Trends Biochem Sci August;18(8). 284-7 (1993)); chromatography (gel filtration chromatography, ion-exchange chromatography); electrophoresis (see, e.g., Ausubel et al., eds. Current Protocols in Molecular Biology, J. Wiley: New York (1999)); and immunoprecipitation (see, e.g., Ausubel et al., eds., supra). Media and chromatographic techniques are known to one skilled in the art (see, e.g., Heegaard, J Mol. Recognit. Winter; 11(1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of target molecule expression are identified. For example, a cell or cell free mixture is contacted with a candidate compound and the expression of target mRNA or RAD21 polypeptide is evaluated relative to the level of expression of target mRNA or RAD21 polypeptide in the absence of the candidate compound. When expression of target mRNA or RAD21 polypeptide is greater in the presence of the candidate compound than in its absence, the candidate compound is identified as an agonist of target mRNA or RAD21 polypeptide expression. Alternatively, when expression of target mRNA or RAD21 polypeptide is less (e.g., less with statistical significance) in the presence of the candidate compound than in its absence, the candidate compound is identified as an antagonist or inhibitor of target mRNA or RAD21 polypeptide expression. The level of target mRNA or RAD21 polypeptide expression can be determined by methods described herein.

In another embodiment, binding partners that interact with a target molecule are detected. The target molecules can interact with one or more cellular or extracellular macromolecules, such as polypeptides in vivo, and these interacting molecules are referred to herein as “binding partners.” Binding partners can agonize or antagonize target molecule biological activity. Also, test molecules that agonize or antagonize interactions between target molecules and binding partners can be useful as therapeutic molecules as they can up-regulate or down-regulated target molecule activity in vivo and thereby treat breast cancer.

Binding partners of target molecules can be identified by methods known in the art. For example, binding partners may be identified by lysing cells and analyzing cell lysates by electrophoretic techniques. Alternatively, a two-hybrid assay or three-hybrid assay can be utilized (see, e.g., U.S. Pat. No. 5,283,317; Zervos et al., Cell 72:223-232 (1993); Madura et al., J. Biol. Chem. 268: 12046-12054 (1993); Bartel et al., Biotechniques 14: 920-924 (1993); Iwabuchi et al., Oncogene 8: 1693-1696 (1993); and Brent WO94/10300). A two-hybrid system is based on the modular nature of most transcription factors, which consist of separable DNA-binding and activation domains. The assay often utilizes two different DNA constructs. In one construct, a nucleic acid from FIGS. 1A-1S or FIG. 2 (sometimes referred to as the “bait”) is fused to a gene encoding the DNA binding domain of a known transcription factor (e.g., GAL-4). In another construct, a DNA sequence from a library of DNA sequences that encodes a potential binding partner (sometimes referred to as the “prey”) is fused to a gene that encodes an activation domain of the known transcription factor. Sometimes, a nucleic acid from FIGS. 1A-1S or FIG. 2 can be fused to the activation domain. If the “bait” and the “prey” molecules interact in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to identify the potential binding partner.

In an embodiment for identifying test molecules that antagonize or agonize complex formation between target molecules and binding partners, a reaction mixture containing the target molecule and the binding partner is prepared, under conditions and for a time sufficient to allow complex formation. The reaction mixture often is provided in the presence or absence of the test molecule. The test molecule can be included initially in the reaction mixture, or can be added at a time subsequent to the addition of the target molecule and its binding partner. Control reaction mixtures are incubated without the test molecule or with a placebo. Formation of any complexes between the target molecule and the binding partner then is detected. Decreased formation of a complex in the reaction mixture containing test molecule as compared to in a control reaction mixture indicates that the molecule antagonizes target molecule/binding partner complex formation. Alternatively, increased formation of a complex in the reaction mixture containing test molecule as compared to in a control reaction mixture, indicates that the molecule agonizes target molecule/binding partner complex formation. In another embodiment, complex formation of target molecule/binding partner can be compared to complex formation of mutant target molecule/binding partner (e.g., amino acid modifications in a RAD21 polypeptide). Such a comparison can be important in those cases where it is desirable to identify test molecules that modulate interactions of mutant but not non-mutated target gene products.

The assays can be conducted in a heterogeneous or homogeneous format. In heterogeneous assays, target molecule and/or the binding partner are immobilized to a solid phase, and complexes are detected on the solid phase at the end of the reaction. In homogeneous assays, the entire reaction is carried out in a liquid phase. In either approach, the order of addition of reactants can be varied to obtain different information about the molecules being tested. For example, test compounds that agonize target molecule/binding partner interactions can be identified by conducting the reaction in the presence of the test molecule in a competition format. Alternatively, test molecules that agonize preformed complexes, e.g., molecules with higher binding constants that displace one of the components from the complex, can be tested by adding the test compound to the reaction mixture after complexes have been formed.

In a heterogeneous assay embodiment, the target molecule or the binding partner is anchored onto a solid surface (e.g., a microtiter plate), while the non-anchored species is labeled, either directly or indirectly. The anchored molecule can be immobilized by non-covalent or covalent attachments. Alternatively, an immobilized antibody specific for the molecule to be anchored can be used to anchor the molecule to the solid surface. The partner of the immobilized species is exposed to the coated surface with or without the test molecule. After the reaction is complete, unreacted components are removed (e.g., by washing) such that a significant portion of any complexes formed will remain immobilized on the solid surface. Where the non-immobilized species is pre-labeled, the detection of label immobilized on the surface is indicative of complex. Where the non-immobilized species is not pre-labeled, an indirect label can be used to detect complexes anchored to the surface; e.g., by using a labeled antibody specific for the initially non-immobilized species. Depending upon the order of addition of reaction components, test compounds that inhibit complex formation or that disrupt preformed complexes can be detected.

In another embodiment, the reaction can be conducted in a liquid phase in the presence or absence of test molecule, where the reaction products are separated from unreacted components, and the complexes are detected (e.g., using an immobilized antibody specific for one of the binding components to anchor any complexes formed in solution, and a labeled antibody specific for the other partner to detect anchored complexes). Again, depending upon the order of addition of reactants to the liquid phase, test compounds that inhibit complex or that disrupt preformed complexes can be identified.

In an alternate embodiment, a homogeneous assay can be utilized. For example, a preformed complex of the target gene product and the interactive cellular or extracellular binding partner-product is prepared. One or both of the target molecule or binding partner is labeled, and the signal generated by the label(s) is quenched upon complex formation (, e.g., U.S. Pat. No. 4,109,496 that-utilizes this approach for immunoassays). Addition of a test molecule that competes with and displaces one of the species from the preformed complex will result in the generation of a signal above background. In this way, test substances that disrupt target molecule/binding partner complexes can be identified.

Identification of Candidate Therapeutics

Candidate therapeutics for treating breast cancer are identified from a group of test molecules that interact with a target molecule. Test molecules are normally ranked according to the degree with which they modulate (e.g., agonize or antagonize) a function associated with the target molecule (e.g., DNA replication and/or processing, RNA transcription and/or processing, polypeptide production and/or processing, and/or biological function/activity), and then top ranking modulators are selected. Also, pharmacogenomic information described herein can determine the rank of a modulator. The top 10% of ranked test molecules often are selected for further testing as candidate therapeutics, and sometimes the top 15%, 20%, or 25% of ranked test molecules are selected for further testing as candidate therapeutics. Candidate therapeutics typically are formulated for administration to a subject.

Therapeutic Formulations

Formulations and pharmaceutical compositions typically include in combination with a pharmaceutically acceptable carrier one or more target molecule modulators. The modulator often is a test molecule identified as having an interaction with a target molecule by a screening method described above. The modulator may be a compound, an antisense nucleic acid, a ribozyme, an antibody, or a binding partner. Also, formulations may comprise a RAD21 polypeptide or fragment thereof in combination with a pharmaceutically acceptable carrier.

As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. Pharmaceutical compositions can be included in a container, pack, or dispenser together with instructions for administration.

A pharmaceutical composition typically is formulated to be compatible with its intended route of administration. Examples of routes of administration include parenteral, e.g., intravenous, intradermal, subcutaneous, oral (e.g., inhalation), transdermal (topical), transmucosal, and rectal administrations Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation often utilized are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices often are utilized. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules lies preferably within a range of circulating concentrations that include the ED₅₀ with little or no toxicity. The dosage may vary within this range depending upon the dosage form employed and the route of administration utilized. For any molecules used in methods described herein, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

For antibodies, a dosage of 0.1 mg/kg of body weight (generally 10 mg/kg to 20 mg/kg) is often utilized. If the antibody is to act in the brain, a dosage of 50 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14:193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

With regard to nucleic acid formulations, gene therapy vectors can be delivered to a subject by, for example, intravenous injection, local administration (see, e.g., U.S. Pat. No. 5,328,470) or by stereotactic injection (see e.g., Chen et al., (1994) Proc. Natl. Acad. Sci. USA 91:3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Therapeutic Methods

A therapeutic formulation described above can be administered to a subject in need of a therapeutic for treating breast cancer. Therapeutic formulations can be administered by any of the paths described herein. With regard to both prophylactic and therapeutic methods of treatment, such treatments may be specifically tailored or modified, based on knowledge obtained from pharmacogenomic analyses described herein.

As used herein, the term “treatment” is defined as the application or administration of a therapeutic formulation to a subject, or application or administration of a therapeutic agent to an isolated tissue or cell line from a subject with the purpose to cure, heal, alleviate, relieve, alter, remedy, ameliorate, improve or affect breast cancer, symptoms of breast cancer or a predisposition towards breast cancer. A therapeutic formulation includes, but is not limited to, small molecules, peptides, antibodies, ribozymes and antisense oligonucleotides. Administration of a therapeutic formulation can occur prior to the manifestation of symptoms characteristic of breast cancer, such that breast cancer is prevented or delayed in its progression. The appropriate therapeutic composition can be determined based on screening assays described herein.

As discussed, successful treatment of breast cancer can be brought about by techniques that serve to agonize target molecule expression or function, or alternatively, antagonize target molecule expression or function. These techniques include administration of modulators that include, but are not limited to, small organic or inorganic molecules; antibodies (including, for example, polyclonal, monoclonal, humanized, anti-idiotypic, chimeric or single chain antibodies, and FAb, F(ab′)₂ and FAb expression library fragments, scFV molecules, and epitope-binding fragments thereof); and peptides, phosphopeptides, or polypeptides.

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above. It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular polypeptide, it can be preferable to co-administer normal target gene polypeptide into the cell or tissue in order to maintain the requisite level of cellular or tissue target gene activity.

Another method by which nucleic acid molecules may be utilized in treating or preventing breast cancer is use of aptamer molecules specific for target molecules. Aptamers are nucleic acid molecules having a tertiary structure which permits them to specifically bind to ligands (see, e.g., Osborne, et al., Curr. Opin. Chem. Biol. 1(1): 5-9 (1997); and Patel, D. J., Curr. Opin. Chem. Biol. June;1(1): 32-46 (1997)).

Yet another method of utilizing nucleic acid molecules for breast cancer treatment is gene therapy, which can also be referred to as allele therapy. Provided herein is a gene therapy method for treating breast cancer in a subject, which comprises contacting one or more cells in the subject or from the subject with a nucleic acid having a first nucleotide sequence. Genomic DNA in the subject comprises a second nucleotide sequence having one or more polymorphic variations associated with breast cancer (e.g., the second nucleic acid is selected from FIGS. 1A-1S or FIG. 2). The first and second nucleotide sequences typically are substantially identical to one another, and the first nucleotide sequence comprises fewer polymorphic variations associated with breast cancer than the second nucleotide sequence. The first nucleotide sequence may comprise a gene sequence that encodes a full-length polypeptide or a fragment thereof. The subject is often a human. Allele therapy methods often are utilized in conjunction with a method of first determining whether a subject has genomic DNA that includes polymorphic variants associated with breast cancer.

In another allele therapy embodiment, provided herein is a method which comprises contacting one or more cells in the subject or from the subject with a polypeptide encoded by a nucleic acid having a first nucleotide sequence. Genomic DNA in the subject comprises a second nucleotide sequence having one or more polymorphic variations associated with breast cancer (e.g., the second nucleic acid is selected from FIGS. 1A-1S or FIG. 2). The first and second nucleotide sequences typically are substantially identical to one another, and the first nucleotide sequence comprises fewer polymorphic variations associated with breast cancer than the second nucleotide sequence. The first nucleotide sequence may comprise a gene sequence that encodes a full-length polypeptide or a fragment thereof. The subject is often a human.

For antibody-based therapies, antibodies can be generated that are both specific for target molecules and that reduce target molecule activity. Such antibodies may be administered in instances where antagonizing a target molecule function is appropriate for the treatment of breast cancer.

In circumstances where stimulating antibody production in an animal or a human subject by injection with a target molecule is harmful to the subject, it is possible to generate an immune response against the target molecule by use of anti-idiotypic antibodies (see, e.g., Herlyn, Ann. Med.;31(1): 66-78 (1999); and Bhattacharya-Chatterjee & Foon, Cancer Treat. Res.; 94: 51-68 (1998)). Introducing an anti-idiotypic antibody to a mammal or human subject often stimulates production of anti-anti-idiotypic antibodies, which typically are specific to the target molecule. Vaccines directed to breast cancer also may be generated in this fashion.

In instances where the target molecule is intracellular and whole antibodies are used, internalizing antibodies often are utilized. Lipofectin or liposomes can be used to deliver the antibody or a fragment of the Fab region that binds to the target antigen into cells. Where fragments of the antibody are used, the smallest inhibitory fragment that binds to the target antigen often is utilized. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see, e.g., Marasco et al., Proc. Natl. Acad. Sci. USA 90: 7889-7893 (1993)).

Modulators can be administered to a patient at therapeutically effective doses to treat breast cancer. A therapeutically effective dose refers to an amount of the modulator sufficient to result in amelioration of symptoms of breast cancer. Toxicity and therapeutic efficacy of modulators can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Modulators that exhibit large therapeutic indices often are utilized. While modulators that exhibit toxic side effects can be used, care should be taken to design a delivery system that targets such molecules to the site of affected tissue in order to minimize potential damage to uninfected cells, thereby reducing side effects.

Data obtained from cell culture assays and animal studies can be used in formulating a range of dosages for use in humans. The dosage of such compounds lies preferably within a range of circulating concentrations that include the ED₅₀ with little or no toxicity. The dosage can vary within this range depending upon the dosage form employed and the route of administration utilized. For any compound used in a method described herein, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. Molecules that modulate target molecule activity are used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding assays, whereby the immobilized monoclonal antibody component is replaced by an appropriately imprinted matrix. An example of the use of such matrixes in this way can be seen in Vlatakis, et al., Nature 361: 645-647 (1993). Through the use of isotope-labeling, the “free” concentration of compound which modulates target molecule expression or activity readily can be monitored and used in calculations of IC₅₀. Such “imprinted” affinity matrixes can also be designed to include fluorescent groups whose photon-emitting properties measurably change upon local and selective binding of target compound. These changes readily can be assayed in real time using appropriate fiberoptic devices, in turn allowing the dose in a test subject to be quickly optimized based on its individual IC₅₀. An example of such a “biosensor” is discussed in Kriz et al., Analytical Chemistry 67: 2142-2144 (1995).

The examples set forth below are intended to illustrate but not limit the invention.

### EXAMPLES

In the following studies a group of subjects were selected according to specific parameters pertaining to breast cancer. Nucleic acid samples obtained from individuals in the study group were subjected to genetic analyses that identified associations between breast cancer and certain polymorphic variants in human genomic DNA. Experiments were performed to investigate the effects of inhibition of RAD21 mRNA expression in selected breast cancer cell lines. Specific siRNA against RAD21 were designed and transfected into breast cells. Gene expression, cell proliferation, apoptosis and sensitivity to chemotherapeutic agents were measured. Methods are described for producing RAD21 polypeptides encoded by the nucleic acids of FIGS. 1A-1S or FIG. 2 in vitro or in vivo, which can be utilized in methods that screen test molecules for those that interact with RAD21 polypeptides. Test molecules identified as being interactors with RAD21 polypeptides can be screened further as breast cancer therapeutics.

### Example 1

### Samples and Pooling Strategies

Sample Selection

Blood samples were collected from individuals diagnosed with breast cancer, which were referred to case samples. Also, blood samples were collected from individuals not diagnosed with breast cancer or any form of cancer or a history of breast cancer; these samples served as gender and age-matched controls. All of the samples were of German/German descent. A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 300×g and the supernatant was carefully poured off. 100-200 μl of residual liquid was left in the tube and was pipetted repeatedly to resuspend the pellet in the residual supernatant. White cell lysis solution (WCL) was added to the tube and pipetted repeatedly until completely mixed. While no incubation was normally required, the solution was incubated at 37° C. or room temperature if cell clumps were visible after mixing until the solution was homogeneous. 2 ml of protein precipitation was added to the cell lysate. The mixtures were vortexed vigorously at high speed for 20 sec to mix the protein precipitation solution uniformly with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1×TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (typically 1:80, 1:160, 1:320, and 1:640 dilutions) so that it would be within the measurable range of standards. 125 μl of diluted DNA was transferred to a clear U-bottom microtiter plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtiter plate. Standards were inverted six times to mix them, and then 50 μl of 1×TE buffer was pipetted into well A1, 1000 ng/ml of standard was pipetted into well A2, 500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 50 μl was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/μl were re-measured for conformation. Samples having measured DNA concentrations of 20 ng/μl or less were re-measured for confirmation.

Pooling Strategies

Samples were placed into one of two groups based on disease status. The two groups were female case samples and female control samples. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria: the sample was derived from an individual characterized as Caucasian; the sample was derived from an individual of German paternal and maternal descent; the database included relevant phenotype information for the individual; case samples were derived from individuals diagnosed with breast cancer; control samples were derived from individuals free of cancer and no family history of breast cancer; and sufficient genomic DNA was extracted from each blood sample for all allelotyping and genotyping reactions performed during the study. Phenotype information included pre- or post-menopausal, familial predisposition, country or origin of mother and father, diagnosis with breast cancer (date of primary diagnosis, age of individual as of primary diagnosis, grade or stage of development, occurrence of metastases, e.g., lymph node metastases, organ metastases), condition of body tissue (skin tissue, breast tissue, ovary tissue, peritoneum tissue and myometrium), method of treatment (surgery, chemotherapy, hormone therapy, radiation therapy). Samples that met these criteria were added to appropriate pools based on gender and disease status.

The selection process yielded the pools set forth in Table 3, which were used in the studies that follow:

TABLE 3

| | Female case | Female control |
|---|---|---|
| Pool size | | |
| (Number) | 272 | 276 |
| Pool Criteria | | |
| (ex: case/control) | case | control |
| Mean Age | | |
| (ex: years) | 59.6 | 55.4 |

### Example 2

### Association of Polymorphic Variants with Breast Cancer

A whole-genome screen was performed to identify particular SNPs associated with occurrence of breast cancer. As described in Example 1, two sets of samples were utilized, which included samples from female individuals having breast cancer (breast cancer cases) and samples from female individuals not having cancer (female controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with breast cancer when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p value was calculated to determine whether the case and control groups had statistically significant differences in allele frequencies for a particular SNP. When the genotyping results agreed with the original allelotyping results, the SNP disease association was considered validated at the genetic level.

SNP Panel Used for Genetic Analyses

A whole-genome SNP screen began with an initial screen of approximately 25,000 SNPs over each set of disease and control samples using a pooling approach. The pools studied in the screen are described in Example 1. The SNPs analyzed in this study were part of a set of 25,488 SNPs confirmed as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according to the following criteria: they are located in ESTs; they are located in Locuslink or Ensembl genes; and they are located in Genomatix promoter predictions. SNPs in the set were also selected on the basis of even spacing across the genome, as depicted in Table 4.

TABLE 4

| General Statistics | | Spacing Statistics | |
|---|---|---|---|
| Total # of SNPs | 25,488 | Median | 37,058 bp |
| # of Exonic SNPs | >4,335 (17%) | Minimum* | 1,000 bp |
| # SNPs with refSNP ID | 20,776 (81%) | Maximum* | 3,000,000 bp |
| Gene Coverage | >10,000 | Mean | 122,412 bp |
| Chromosome Coverage | All | Std Deviation | 373,325 bp |

*Excludes outliers

Allelotyping and Genotyping Results

The genetic studies summarized above and described in more detail below identified allelic variants associated with breast cancer, which are summarized in Table 2.

Assay for Verifying, Allelotyping, and Genotyoing SNPs

A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND™ (Sequenom, Inc.)) in which two genotyping primers anneal to and amplify a genomic target surrounding a polymorphic site of interest. A third primer (the MassEXTEND™ primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND™ primer which where used to genotype the polymorphism. Other primer design software could be used or one of ordinary skill in the art could manually design primers based on his or her knowledge of the relevant factors and considerations in designing such primers. Table 5 shows PCR primers and Table 6 shows extension primers used for analyzing polymorphisms. The initial PCR amplification reaction was performed in a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

TABLE 5
PCR Primers

SNP
Refer-

| | | |
|---|---|---|
| ence | Forward PCR primer | Reverse PCR primer |

| | | |
|---|---|---|
| rs- | ATACCTGTGGCGTACACATG | AAAAGGTAGGCCTCACTTGC |
| 1374297 | | |

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND™ primer cocktail to each sample. Each MassEXTEND™ cocktail included a specific combination of dideoxynucleotides (ddNTPs) and deoxynucleotides (dNTPs) used to distinguish polymorphic alleles from one another. Methods for verifying, allelotyping and genotyping SNPs are disclosed, for example, in U.S. Pat. No. 6,258,538, the content of which is hereby incorporated by reference. In Table 6, ddNTPs are shown and the fourth nucleotide not shown is the dNTP.

TABLE 6
Extension Primers

| | |
|---|---|
| SNP | Termination |

| Reference | Extend Probe | Mix |
|---|---|---|
| rs1374297 | CTGTGGCGTACACATGAAACTG | ACT |

The MassEXTEND™ reaction was performed in a total volume of 9 μl, with the addition of 1× ThermoSequenase buffer, 0.576 units of ThermoSequenase (Amersham Pharmacia), 600 nM MassEXTEND™ primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C, and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJE™ (Sequenom, Inc.)) onto either 96-spot or 384-spot silicon chips containing a matrix that crystallized each sample (SpectroCHIP™ (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

Minor allelic frequencies for the polymorphisms set forth in FIG. 1 were verified as being 10% or greater using the extension assay described above in a group of samples isolated from 92 individuals originating from the state of Utah in the United States, Venezuela and France (Coriell cell repositories).

Table 7 shows allelotyping results in female breast cancer and female control pools. Allele frequency is noted in the second and third columns for breast cancer pools and control pools, respectively, and the allele indicated in bold type is the dominant allele. Genotyping results are shown for female pools in Table 8. In the subsequent tables, “AF” refers to allelic frequency; and “F case” and “F control” refer to female case and female control groups, respectively.

TABLE 7
Allelotyping Results

| | AF | AF | |
|---|---|---|---|
| SNP Reference | F case | F control | p-value |
| rs1374297 | C = 0.792 | C = 0.881 | 0.0003 |
| | G = 0.208 | G = 0.119 | |

TABLE 8
Genotyping Results

| | AF | AF | |
|---|---|---|---|
| SNP Reference | F case | F control | p-value |
| | C = 0.852 | C = 0.925 | 0.0004 |
| rs1374297 | G = 0.148 | G = 0.075 | |

The single marker alleles set forth in Table 2 were considered validated, since the genotyping data agreed with the allelotyping data and each SNP significantly associated with breast cancer. Particularly significant associations with breast cancer are indicated by a calculated p-value of less than 0.04 for allelotype results and a calculated p-value of less than 0.05 for genotype results, which are set forth in bold text.

### Example 3

### RAD21 Proximal SNPs

It has been discovered that a polymorphic variation in a gene encoding RAD21 is associated with the occurence of breast cancer (see Examples 1 and 2). SNPs proximal to the incident SNP (rs1374297) were identified and allelotyped in breast cancer sample sets and control sample sets as described in Examples 1 and 2. A total of fifty-eight allelic variants located within or nearby the RAD21 gene were identified and subsequently allelotyped. The polymorphic variants are set forth in Table 9. the chromosome position provided in column four of Table 9 is based on Genome “Build 31” of NCBI's GenBank. The gene position provided in column five of Table 9 refers to the position (e.g., intronic or exonic) of each SNP within or relative to the RAD21 gene.

TABLE 9

| dbSNP | Chro- | Position in | Chromosome | Gene | Allele |
|---|---|---|---|---|---|
| rs# | mosome | | Position | Position | Variants |
| 2053728 | 8 | 56 | 117912256 | locus-region | G/C |
| 63184 | 8 | 7324 | 117919524 | intragenic | G/A |
| 48390 | 8 | 7363 | 117919563 | intragenic | A/G |
| 1348799 | 8 | 9231 | 117921431 | intron | A/T |
| 2289938 | 8 | 10490 | 117922690 | intron | G/A |
| 1063547 | 8 | 11867 | 117924067 | coding- | A/G |
| | | | | synon | |
| 2921790 | 8 | 12308 | 117924508 | intron | T/A |
| 1050838 | 8 | 13191 | 117925391 | coding- | C/T |
| | | | | synon | |
| 2241982 | 8 | 13525 | 117925725 | intron | G/A |
| 2241981 | 8 | 13582 | 117925782 | intron | T/G |
| 3020181 | 8 | 15875 | 117928075 | intron | G/A |
| 3020111 | 8 | 17237 | 117929437 | intron | G/C |
| 2921787 | 8 | 18472 | 117930672 | intron | A/G |
| 3020117 | 8 | 19170 | 117931370 | intron | A/C |
| 3816342 | 8 | 19349 | 117931549 | intron | C/T |
| 2921785 | 8 | 23869 | 117936069 | intron | T/A |
| 2921784 | 8 | 24235 | 117936435 | intron | T/A |
| 3020125 | 8 | 26691 | 117938891 | intron | G/A |
| 3020130 | 8 | 31373 | 117943573 | mrna-utr | A/T |
| 2921782 | 8 | 31979 | 117944179 | mrna-utr | C/G |
| 3020131 | 8 | 33472 | 117945672 | mrna-utr | T/C |
| 1374298 | 8 | 33545 | 117945745 | mrna-utr | T/G |
| 1374297 | 8 | 33670 | 117945870 | intragonic | G/C |
| 3020132 | 8 | 33778 | 117945978 | mrna-utr | T/C |
| 1348798 | 8 | 34691 | 117946891 | mrna-utr | A/T |
| 1838839 | 8 | 36859 | 117949059 | locus-region | G/A |
| 3020138 | 8 | 47651 | 117959851 | | A/G |
| 1348796 | 8 | 48463 | 117960663 | | G/A |
| 3020139 | 8 | 49058 | 117961258 | | A/C |
| 1374296 | 8 | 50233 | 117962433 | | C/A |
| 2921771 | 8 | 51228 | 117963428 | | A/G |
| 1348795 | 8 | 52315 | 117964515 | | A/G |
| 3020141 | 8 | 53039 | 117965239 | | A/G |
| 1374295 | 8 | 62940 | 117975140 | | G/T |
| 2921755 | 8 | 64531 | 117976731 | | T/C |
| 720131 | 8 | 64989 | 117977189 | | A/G |
| 720132 | 8 | 65209 | 117977409 | | T/C |
| 3020152 | 8 | 65444 | 117977644 | | A/C |
| 2921750 | 8 | 70056 | 117982256 | | G/T |
| 3020156 | 8 | 70329 | 117982529 | | A/G |
| 3020159 | 8 | 70629 | 117982829 | | A/G |
| 2921748 | 8 | 71326 | 117983526 | | G/A |
| 3020160 | 8 | 72563 | 117984763 | | T/A |
| 1466029 | 8 | 73135 | 117985335 | | C/T |
| 3020161 | 8 | 73627 | 117985827 | | G/A |
| 2317383 | 8 | 74621 | 117986821 | | C/T |
| 3020163 | 8 | 75303 | 117987503 | | A/G |
| 3020164 | 8 | 75749 | 117987949 | | T/G |
| 2921745 | 8 | 75855 | 117988055 | | G/A |
| 3020167 | 8 | 77799 | 117989999 | | C/T |
| 2921739 | 8 | 78432 | 117990632 | | T/C |
| 2921738 | 8 | 78648 | 117990848 | | G/C |
| 2921737 | 8 | 79585 | 117991785 | | G/A |
| 3020168 | 8 | 79791 | 117991991 | | C/T |
| 3020169 | 8 | 80037 | 117992237 | | A/G |
| 1020405 | 8 | 80082 | 117992282 | | A/T |
| 2921735 | 8 | 82490 | 117994690 | | G/A |
| 2921734 | 8 | 83324 | 117995524 | | C/T |

Assay for Verifying and Allelotyping SNPs

The methods used to verify and allelotype the fifty-eight proximal SNPs of Table 9 are the same methods described in Examples 1 and 2 herein. The PCR primers and extend primers used in these assays are provided in Table 10 and Table 11, respectively.

TABLE 10

| | | |
|---|---|---|
| dbSNP | Forward | Reverse |

| | | |
|---|---|---|
| rs# | PCR primer | PCR primer |

| | | |
|---|---|---|
| 48390 | ACGTTGGATGACAAACGGGGAAAACTCCTT | ACGTTGGATGAATGATTCAGTTTCTTCAGAGTGGT |
| 63184 | ACGTTGGATGTTCAATATGATGTGCCTGTAAACC | ACGTTGGATGTGACCTTTCTAAAATCAAACATTCA |
| 720131 | ACGTTGGATGTGGATTCATTCCATGCGAGC | ACGTTGGATGGCAAGTGCATGGACAATGAG |
| 720132 | ACGTTGGATGGAGAATGCATAGTCTATCTG | ACGTTGGATGACCCTAGACACTCCTTACTC |
| 1020405 | ACGTTGGATGCACTGGTTAATTGCTGTTTC | ACGTTGGATGAGCATGTGTCAACTAAGAGG |
| 1050838 | ACGTTGGATGATAGATGAGTCAGCTATGCC | ACGTTGGATGTACTTACAGGCATCACAGGC |
| 1063547 | ACGTTGGATGCCAGAGTTAGAACTTCTGCC | ACGTTGGATGGCATCTTCATCCTCTTCCTC |
| 1348795 | ACGTTGGATGAGTGAAATTTCCATGCCCTC | ACGTTGGATGGTGTTCAGAAAGGCTTCTGG |
| 1348796 | ACGTTGGATGAATAGGATTAACTAAGAAGC | ACGTTGGATGCTCAGCTACAGAGGTAATAG |
| 1348798 | ACGTTGGATGTTGAGAAACCTTCTCCTGCC | ACGTTGGATGCTTAAATTGGGTGTAAATGCC |
| 1348799 | ACGTTGGATGTTGCCATGTGACACACCTGC | ACGTTGGATGAAAGCACCAGCATCTGCTTC |
| 1357572 | ACGTTGGATGCCCTGAGAAGTTTAAGCTTG | ACGTTGGATGGCAAGGTAAGAGGATACAAG |
| 1374295 | ACGTTGGATGTGTAAGATGCACGAGGACAG | ACGTTGGATGACACCTGTCGACTAACTTTC |
| 1374296 | ACGTTGGATGAATTCCACAGCCAGACACAC | ACGTTGGATGTGAGTATCAAGCTGTTTGAC |
| 1374298 | ACGTTGGATGTTTTTGCACTTAACCTGGAG | ACGTTGGATGCAGTACAACTTTAAACAAG |
| 1466029 | ACGTTGGATGAATGGAGTCTGAAGGCCATG | ACGTTGGATGGTTTTGGTTTAATTCCTGAG |
| 1838839 | ACGTTGGATGGGAACCACAATAAGACCAAG | ACGTTGGATGTACTGAGCCTTGAAGGATGC |
| 2053728 | ACGTTGGATGAACCATCACCCATACTGTCC | ACGTTGGATGTACTGAGCCTTGAAGGATGC |
| 1374297 | ACGTTGGATGATACCTGTGGCGTACACATG | ACGTTGGATGAAAAGGTAGGCCTCACTTGC |
| 2241981 | ACGTTGGATGGCAGGGAAATGCATTGGATC | ACGTTGGATGACTATCTACCCTGCCAGTTC |
| 2241982 | ACGTTGGATGGGAAAGGGGATCTTAAAAGG | ACGTTGGATGAACTGGCAGGGTAGATAGTC |
| 2289938 | ACGTTGGATGCAAAGTCCTCTATGTGCAAG | ACGTTGGATGAGTGTGTGTAGATAGCATCC |
| 2317383 | ACGTTGGATGGCGGCGACTGATTGTGCTAC | ACGTTGGATGTCTCCTGATCCATGGGTTGC |
| 2921734 | ACGTTGGATGTTGGGATTACAGGTGTGAGC | ACGTTGGATGCTGGGTAGTGAAATTGGGTG |
| 2921735 | ACGTTGGATGGCAAGCTCACATGCGTGTAG | ACGTTGGATGGACTATTCTGTAGTCTGTGTG |
| 2921737 | ACGTTGGATGGATGAGTAGAGTTGAGTTCC | ACGTTGGATGGCTCAGGGCAAGAAAGAATC |
| 2921738 | ACGTTGGATGGTCAAGCTCAAGAGTGGAAG | ACGTTGGATGTTTAACCCCACATAGCAGCC |
| 2921739 | ACGTTGGATGTCCCATCTCACAAAGCAACC | ACGTTGGATGAAGTGAGCAACTGAGTCCTC |
| 2921745 | ACGTTGGATGTGCTTGCATCAGAGTGTTTC | ACGTTGGATGTTTGCCAAAATCTCTTGTGC |
| 2921748 | ACGTTGGATGCACTAGAGGAAAACCTAGGC | ACGTTGGATGTAGACACAAAGTCCTTGCCC |
| 2921750 | ACGTTGGATGAGGCCAAGATTGGTTTTGAC | ACGTTGGATGTCGCTGAATCCCATGAAGAC |
| 2921755 | ACGTTGGATGAGAGAGGAAGGAGGAGAAAC | ACGTTGGATGCTCAGAGTGGTAGGAAATCC |
| 2921771 | ACGTTGGATGCAAATGAAGTTGGAGAGAGC | ACGTTGGATGACTTTGCATTGCTAACTTTC |
| 2921782 | ACGTTGGATGGCAAGCAACTGTATCCTAAAC | ACGTTGGATGGATCACTTGGTGGATCTTAC |
| 2921784 | ACGTTGGATGGTGTTACTGTAGCTAAACACA | ACGTTGGATGTATCTTTGAAGGGTTCCTCG |
| 2921785 | ACGTTGGATGAACTGGAGTCTGCCAACCAC | ACGTTGGATGCAGTAGAAACTGTTTAAGGC |
| 2921787 | ACGTTGGATGGGAGAAGGAAATGATGGTGG | ACGTTGGATGCTGTTTATGCTGGAATAACC |
| 2921790 | ACGTTGGATGTTTGCTGCCGTGAGACATTC | ACGTTGGATGCTACTAAAGCTTCTGTAAGG |
| 3020111 | ACGTTGGATGTTCTGTTTTTTTGGCCTGTC | ACGTTGGATGCTATGACAGATGACTGTGAC |
| 3020117 | ACGTTGGATGATTGTTTTTTAAGAGGCGGG | ACGTTGGATGGTGCATAATCCAGCCTGTG |
| 3020125 | ACGTTGGATGCAGTTTGTTCTGGTGAGATC | ACGTTGGATGCTTATCCCAGTAAGCATACC |
| 3020130 | ACGTTGGATGAGACAGTTGACAAAGCCTGG | ACGTTGGATGTCTCTGAATCTAATGTTCCC |
| 3020131 | ACGTTGGATGGTTGTACTGTACAATTGTCCC | ACGTTGGATGAAGCGACTTGAGCATTCGTG |
| 3020132 | ACGTTGGATGTGGTGTACATTTATGTCCCG | ACGTTGGATGTGAGGCCTACCTTTTTGTAC |
| 3020138 | ACGTTGGATGGTTGAGCATCTTTTCATGTG | ACGTTGGATGTGGGCAAAGGACTTGCATAG |
| 3020139 | ACGTTGGATGGTAATCACACTGCTACCCTG | ACGTTGGATGGATTTGTGATTCTTTGAGGG |
| 3020141 | ACGTTGGATGGTGTAGGAAATGGGATTACAG | ACGTTGGATGTATCAAGCCTGGGGTATTCC |
| 3020152 | ACGTTGGATGCAAAGTCATCTGTCCTAACC | ACGTTGGATGCAGGTACTCAATAGATGTGG |
| 3020156 | ACGTTGGATGGTATTCCACATAAGTACTCCC | ACGTTGGATGACAGAAAGCATTTAACAGGG |
| 3020160 | ACGTTGGATGACCTAAAAGACCTGCCACAC | ACGTTGGATGCCTCATGAATTACCTTCTTC |
| 3020161 | ACGTTGGATGTGCCTCTTCTCCTCCAAATG | ACGTTGGATGAGGAACCTGTGCAACTGTAG |
| 3020163 | ACGTTGGATGAACCAAAAGATTCTCTGCTG | ACGTTGGATGATCCCCCAAGCTTGTTACAG |
| 3020164 | ACGTTGGATGGTGATTGGTTCAGGTATGGG | ACGTTGGATGAAACTTGCCCCAGAATCCAC |
| 3020167 | ACGTTGGATGGACCTATACAGGGCACTTAC | ACGTTGGATGCTCACTACTCACACACTGAC |
| 3020168 | ACGTTGGATGTGGAATGTCACCCATGTGAG | ACGTTGGATGACCTGATTTTGAGTCAGTGC |
| 3020169 | ACGTTGGATGGAGGAACAGTCAATGAAGGC | ACGTTGGATGAGCATGTGTCAACTAAGAGG |
| 3020181 | ACGTTGGATGTTGGCCCTTGCGTCATTTTG | ACGTTGGATGCCAACCACCATTCAGAAGAG |
| 3816342 | ACGTTGGATGCCTACTTCTCTCCCTATATG | ACGTTGGATGAATGTTGGGACTCCTCGCAG |

TABLE 11

| | | |
|---|---|---|
| dbSNP | Extend | Term |

| | | |
|---|---|---|
| rs# | Primer | Mix |

| | | |
|---|---|---|
| 48390 | AGGCACATCATATTGAAT | ACT |
| 63184 | AAACCAAGGAGTTTTCCC | ACG |
| 720131 | GAGCTAACTTGGCCTCC | ACT |
| 720132 | TATCCTAATTTCCTTGAGCAC | ACT |
| 1020405 | CCATTCAATTTGTAAAATTTCG | CGT |
| 1050838 | GGAGTTAAGCGAAAAGC | ACG |
| 1063547 | CCAGAAAAAGAGAAGGA | ACT |
| 1348795 | CCCTCCAGACACCTCCAC | ACT |
| 1348796 | AACTAAGAAGCAATAAGGAGAA | ACG |
| 1348798 | CAAAATTCTATAGACTCGCAC | CGT |
| 1348799 | CCCCCTTTGCCTTCCACC | CGT |
| 1357572 | TTCCCCCAAGAAATCAACCC | ACT |
| 1374295 | CGAGGACAGAGACTGTA | CGT |
| 1374296 | AGACACACTGCCCCCCC | CGT |
| 1374298 | CTGGAGATTTTCCATGTTAG | ACT |
| 1466029 | GAAGGCCATGTGAGTATT | ACG |
| 1838839 | GACCAAGAATAGCCAAAG | ACG |
| 2053728 | CTTGCCACTCTCCTTTC | ACT |
| 1374297 | CTGTGGCGTACACATGAAACTG | ACT |
| 2241981 | GCCTCCTGTCTTTCCAGAG | ACT |
| 2241982 | ACAAGTCCTACCCTCAG | ACG |
| 2289938 | TTTGGCTGAAAGTATGCTTCTATA | ACG |
| 2317383 | CGCCTGGGAAACCATGCTT | ACG |
| 2921734 | GTGTGAGCCACTGTGCC | ACG |
| 2921735 | ACCCCCCAAAATGTTTA | ACG |
| 2921737 | AGTTGAGTTCCTTATAAAGAAA | ACG |
| 2921738 | ACTTATTGGCCTCTTAAAAC | ACT |
| 2921739 | CCTCAGTGAATTAAAACTCATCA | ACT |
| 2921745 | TCAGAGTGTTTCTGATTTAAA | ACG |
| 2921748 | GAAAACCTAGGCAATACCA | ACG |
| 2921750 | CAGTTCACTCGTTGATTTA | CGT |
| 2921755 | AGGAGAAACAGGAAAGTACAG | ACT |
| 2921771 | AGAGGATGAATAGGCCC | ACT |
| 2921782 | AAGCTTCTAGAATACTATCTGT | ACT |
| 2921784 | TTTTCTAAATCTACATGCTTTGTT | CGT |
| 2921785 | CCACACCACCATCTAAG | CGT |
| 2921787 | GGTGGAATATTAGGTATGTG | ACT |
| 2921790 | CATTCAAGACTCTCAGAG | CGT |
| 3020111 | TTGGCCTGTCTACTGAT | ACT |
| 3020117 | TCTCTGCTGTGTTATCCA | ACT |
| 3020125 | CATACCAGTTTGCACTGC | ACG |
| 3020130 | AAGCCTGGTTTTTTTTCTTTTG | CGT |
| 3020131 | AAGGGGAATTGGTTCCAG | ACT |
| 3020132 | TTTATGTCCCGAGTTAAAATAT | ACT |
| 3020138 | TTTCATGTGCTTATTGGCC | ACT |
| 3020139 | TCCTCATAAACCATCTTTTT | ACT |
| 3020141 | ATGGGATTACAGAAAATTGAC | ACT |
| 3020152 | TGTCCTAACCACTACAC | ACT |
| 3020156 | TAGAATTCAAAACAAGTGGTAA | ACT |
| 3020160 | CAAAATGATAACACATCAATGTA | CGT |
| 3020161 | TCCAAATGATCTCAACACCT | ACG |
| 3020163 | TCTCTGCTGAAGTTGCT | ACT |
| 3020164 | GATCCAATTCTGGCCAATTAAAT | ACT |
| 3020167 | GCGGCAGGACTGGAACG | ACG |
| 3020168 | AGGGAAAAGAAGACAAATTAAGAC | ACG |
| 3020169 | AAAAAAAAAACACAAAACACTG | ACT |
| 3020181 | CAAATTTTTGTTGAATGCC | ACG |
| 3816342 | CTCTCCCTATATGCAATCA | ACG |

Genetic Analysis

Allelotyping results are shown for cases and controls in Table 12. Allele frequency is noted in the fourth and fifth columns for breast cancer pools and control pools.

TABLE 12

| | | | | | | Disease |
|---|---|---|---|---|---|---|
| Reference | Chrom | | | | | Associated |
| SNP ID | Position | Alleles | Case AF | Control AF | p-Value | Allele |
| 2053728 | 117912256 | G/C | G = 0.572 | G = 0.575 | 0.9208 | |
| | | | C = 0.428 | C = 0.425 | | |
| 63184 | 117919524 | G/A | G = 0.699 | G = 0.723 | 0.3719 | |
| | | | A = 0.301 | A = 0.277 | | |
| | 117919524 | C/T | C = 0.984 | C = 0.987 | 0.7414 | |
| | | | T = 0.016 | T = 0.013 | | |
| 48390 | 117919563 | A/G | A = 0.605 | A = 0.605 | 0.9889 | |
| | | | G = 0.395 | G = 0.395 | | |
| 1348799 | 117921431 | A/T | A = 0.207 | A = 0.212 | 0.8247 | |
| | | | T = 0.793 | T = 0.788 | | |
| 2289938 | 117922690 | G/A | G = 0.273 | G = 0.298 | 0.3618 | |
| | | | A = 0.727 | A = 0.702 | | |
| 1063547 | 117924067 | A/G | A = 0.007 | A = 0.005 | 0.5985 | |
| | | | G = 0.993 | G = 0.995 | | |
| 2921790 | 117924508 | T/A | T = 0.279 | T = 0.269 | 0.7102 | |
| | | | A = 0.721 | A = 0.731 | | |
| 1050838 | 117925391 | C/T | C = 0.233 | C = 0.262 | 0.2677 | |
| | | | T = 0.767 | T = 0.738 | | |
| 2241982 | 117925725 | G/A | G = 0.020 | G = 0.037 | 0.1006 | |
| | | | A = 0.980 | A = 0.963 | | |
| 2241981 | 117925782 | T/G | T = 0.991 | T = 0.987 | 0.6191 | |
| | | | G = 0.009 | G = 0.013 | | |
| 3020181 | 117928075 | G/A | G = 0.992 | G = 0.995 | 0.4529 | |
| | | | A = 0.008 | A = 0.005 | | |
| 3020111 | 117929437 | G/C | G = 0.996 | G = 0.982 | 0.0287 | G |
| | | | C = 0.004 | C = 0.018 | | |
| 2921787 | 117930672 | A/G | A = 0.039 | A = 0.068 | 0.0316 | |
| | | | G = 0.961 | G = 0.932 | | |
| 3020117 | 117931370 | A/C | A = 0.765 | A = 0.775 | 0.6842 | |
| | | | C = 0.236 | C = 0.225 | | |
| 3816342 | 117931549 | C/T | C = 0.210 | C = 0.253 | 0.0931 | |
| | | | T = 0.790 | T = 0.747 | | |
| 2921785 | 117936069 | T/A | T = 0.047 | T = 0.058 | 0.4144 | |
| | | | A = 0.953 | A = 0.942 | | |
| 2921784 | 117936435 | T/A | T = 0.997 | T = 0.993 | 0.3623 | |
| | | | A = 0.003 | A = 0.007 | | |
| 3020125 | 117938891 | G/A | G = 0.016 | G = 0.022 | 0.4439 | |
| | | | A = 0.984 | A = 0.978 | | |
| 3020130 | 117943573 | A/T | A = 0.021 | A = 0.022 | 0.9277 | |
| | | | T = 0.979 | T = 0.978 | | |
| 2921782 | 117944179 | C/G | C = 0.052 | C = 0.076 | 0.1081 | |
| | | | G = 0.948 | G = 0.924 | | |
| 3020131 | 117945672 | T/C | T = 0.976 | T = 0.986 | 0.2397 | |
| | | | C = 0.024 | C = 0.014 | | |
| 1374298 | 117945745 | T/G | T = 0.855 | T = 0.929 | 0.0002 | G |
| | | | G = 0.146 | G = 0.071 | | |
| 1374297 | 117945870 | G/C | G = 0.202 | G = 0.107 | 0.0001 | G |
| | | | C = 0.798 | C = 0.893 | | |
| 3020132 | 117945978 | T/C | T = 0.033 | T = 0.042 | 0.4534 | |
| | | | C = 0.967 | C = 0.958 | | |
| 1348798 | 117946891 | A/T | A = 0.513 | A = 0.530 | 0.5615 | |
| | | | T = 0.487 | T = 0.470 | | |
| 1838839 | 117949059 | G/A | G = 0.977 | G = 0.982 | 0.6189 | |
| | | | A = 0.023 | A = 0.018 | | |
| 3020138 | 117959851 | A/G | A = 0.553 | A = 0.609 | 0.0599 | |
| | | | G = 0.447 | G = 0.391 | | |
| 1348796 | 117960663 | G/A | G = 0.771 | G = 0.759 | 0.6413 | |
| | | | A = 0.229 | A = 0.241 | | |
| 3020139 | 117961258 | A/C | A = 0.042 | A = 0.042 | 0.9783 | |
| | | | C = 0.958 | C = 0.958 | | |
| 1374296 | 117962433 | C/A | C = 0.486 | C = 0.484 | 0.9367 | |
| | | | A = 0.514 | A = 0.516 | | |
| 2921771 | 117963428 | A/G | A = 0.045 | A = 0.062 | 0.2027 | |
| | | | G = 0.955 | G = 0.938 | | |
| 1348795 | 117964515 | A/G | A = 0.625 | A = 0.631 | 0.8374 | |
| | | | G = 0.375 | G = 0.369 | | |
| 3020141 | 117965239 | A/G | A = 0.708 | A = 0.671 | 0.1824 | |
| | | | G = 0.292 | G = 0.329 | | |
| 1374295 | 117975140 | G/T | G = 0.559 | G = 0.559 | 0.9883 | |
| | | | T = 0.441 | T = 0.441 | | |
| 2921755 | 117976731 | T/C | T = 0.754 | T = 0.775 | 0.4091 | |
| | | | C = 0.246 | C = 0.225 | | |
| 720131 | 117977189 | A/G | A = 0.432 | A = 0.399 | 0.2679 | |
| | | | G = 0.568 | G = 0.601 | | |
| 720132 | 117977409 | T/C | T = 0.705 | T = 0.736 | 0.2584 | |
| | | | C = 0.295 | C = 0.264 | | |
| 3020152 | 117977644 | A/C | A = 0.690 | A = 0.673 | 0.5454 | |
| | | | C = 0.310 | C = 0.327 | | |
| 2921750 | 117982256 | G/T | G = 0.740 | G = 0.773 | 0.2136 | |
| | | | T = 0.260 | T = 0.227 | | |
| 3020156 | 117982529 | A/G | A = 0.442 | A = 0.427 | 0.6158 | |
| | | | G = 0.558 | G = 0.573 | | |
| 2921748 | 117983526 | G/A | G = 0.033 | G = 0.025 | 0.4529 | |
| | | | A = 0.967 | A = 0.975 | | |
| 3020160 | 117984763 | T/A | T = 0.441 | T = 0.414 | 0.3789 | |
| | | | A = 0.559 | A = 0.586 | | |
| 1466029 | 117985335 | C/T | C = 0.007 | C = 0.005 | 0.7775 | |
| | | | T = 0.993 | T = 0.995 | | |
| 3020161 | 117985827 | G/A | G = 0.459 | G = 0.436 | 0.4434 | |
| | | | A = 0.541 | A = 0.564 | | |
| 2317383 | 117986821 | C/T | C = 0.589 | C = 0.583 | 0.8397 | |
| | | | T = 0.411 | T = 0.417 | | |
| 3020163 | 117987503 | A/G | A = 0.723 | A = 0.747 | 0.3617 | |
| | | | G = 0.277 | G = 0.253 | | |
| 3020164 | 117987949 | T/G | T = 0.720 | T = 0.732 | 0.6346 | |
| | | | G = 0.280 | G = 0.268 | | |
| 2921745 | 117988055 | G/A | G = 0.298 | G = 0.343 | 0.1105 | |
| | | | A = 0.702 | A = 0.657 | | |
| 3020167 | 117989999 | C/T | C = 0.799 | C = 0.780 | 0.4595 | |
| | | | T = 0.201 | T = 0.220 | | |
| 2921739 | 117990632 | T/C | T = 0.427 | T = 0.422 | 0.8850 | |
| | | | C = 0.573 | C = 0.578 | | |
| 2921738 | 117990848 | G/C | G = 0.440 | G = 0.439 | 0.9781 | |
| | | | C = 0.560 | C = 0.561 | | |
| 2921737 | 117991785 | G/A | G = 0.019 | G = 0.016 | 0.6906 | |
| | | | A = 0.981 | A = 0.984 | | |
| 3020168 | 117991991 | C/T | C = 0.692 | C = 0.693 | 0.9559 | |
| | | | T = 0.308 | T = 0.307 | | |
| 3020169 | 117992237 | A/G | A = 0.015 | A = 0.022 | 0.3583 | |
| | | | G = 0.985 | G = 0.978 | | |
| 1020405 | 117992282 | A/T | A = 0.292 | A = 0.312 | 0.4549 | |
| | | | T = 0.708 | T = 0.688 | | |
| 2921735 | 117994690 | G/A | G = 0.255 | G = 0.298 | 0.1111 | |
| | | | A = 0.745 | A = 0.702 | | |
| 2921734 | 117995524 | C/T | C = 0.822 | C = 0.806 | 0.4958 | |
| | | | T = 0.178 | T = 0.194 | | |

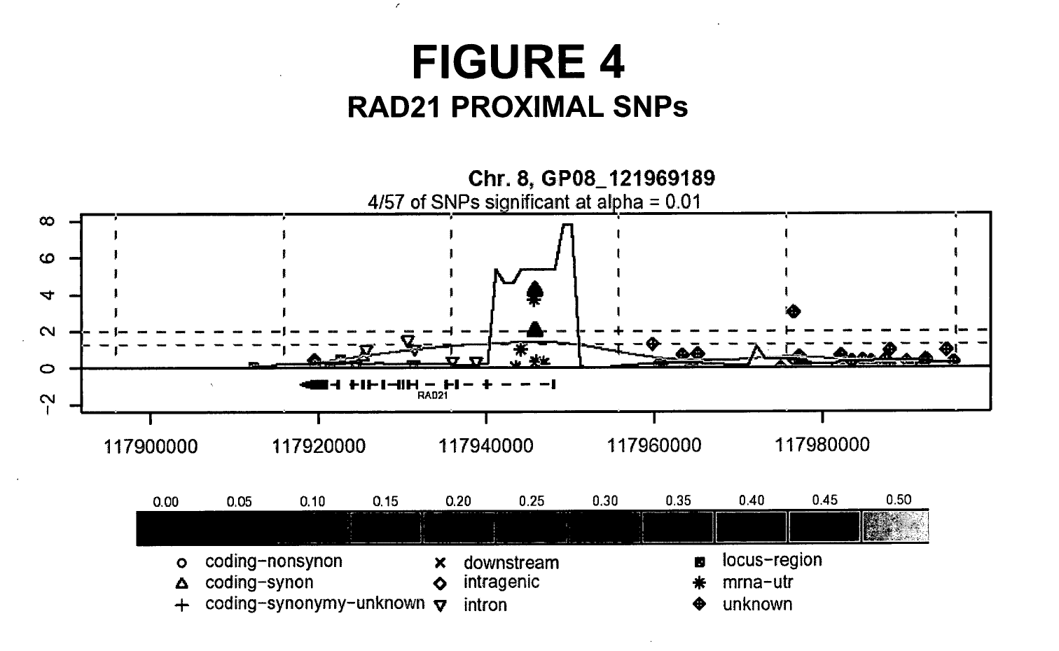

Allelotyping results were considered particularly significant with a calculated p-value of less than or equal to 0.05 for allelotype results. These values are indicated in bold. The allelotyping p-values were plotted in FIG. 4. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graph in FIG. 4 can be determined by consulting Table 12. For example, the left-most X on the left graph is at chromosome position 117912256. Table 12 indicates that the minor allele frequency is 0.425. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear trends in the association of SNPs to the disease. The light gray line (or generally bottom-most curve) is a nonlinear smoother through the data points on the graph using a local polynomial regression method (W.S. Cleveland, E. Grosse and W. M. Shyu (1992) Local regression models. Chapter 8 of Statistical Models in S eds J. M. Chambers and T. J. Hastie, Wadsworth & Brooks/Cole.). The black line (or generally top-most curve, e.g., see peak in left-most graph just to the left of position 92150000) provides a local test for excess statistical significance to identify regions of association. This was created by use of a 10 kb sliding window with 1 kb step sizes. Within each window, a chi-square goodness of fit test was applied to compare the proportion of SNPs that were particularly significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted below each graph at the appropriate chromosomal positions. The gene boundary is indicated by the broken horizontal line. The exon positions are shown as thick, unbroken bars. An arrow is place at the 3′ end of each gene to show the direction of transcription.

Deep Sequencing Reveals Novel SNPs in RAD21

Deep sequencing was performed on all RAD21 exons (including intron/exon boundaries) and 1 kb of the promoter in 6 samples (4 cases homozygote for the disease allele and 2 controls homozygote for the non-disease allele). The primers listed in Table 13 were used to perform the sequencing. In Table 13, the Primer Name identifies the orientation (forward “F” or reverse “R”) and the exon amplified. For example, RAD21F13 is the forward primer for exon 13.

TABLE 13

| | |
|---|---|
| Primer Name | Primer Sequence |

| | |
|---|---|
| RAD21F13 | CTTGGGGTGCTGTTTTCT |
| RAD21R13 | ATTGCCACAGGGAGTGAT |
| RAD21F12 | CTCTCCCTCCAGAAAAAATA |
| RAD21R12 | CTCAGCAGCATTAAGTACAGT |
| RAD21F11 | GAGTTACAGCGAAGCATAA |
| RAD21R11 | TCCTTGTGGGGAAGTATAG |
| RAD21F10 | TGGAGCACTCTAAAGCAATAC |
| RAD21R10 | ATCCCCTTTCCCCTTTAC |
| RAD21F9 | AAGACAGGAGGCTTCATACT |
| RAD21R9 | CCTTTGGAAGATAGAAATCAGT |
| RAD21F8 | AAAGAAAATGTGCCATACAG |
| RAD21R8 | TGCGTCATTTTGCTTATTT |
| RAD21F7 | AAAAAAGCAAGAAGCCTAGT |
| RAD21R7 | TTTCTCCTCCCCATTTGT |
| RAD21F6 | TACAATCATCCCCAGAATC |
| RAD21R6 | CTGGAGGAGAAACAGATAAA |
| RAD21F5 | CCGAAATGTCCTATTGAAC |
| RAD21R5 | TGCCCCAGTGTTGTAACT |
| RAD21F4 | ACTCCTCGCAGAAATCAA |
| RAD21R4 | CTTGGATTGTACTGGAATGTG |
| RAD21F3 | ACAAGCGTATCTGTTTCAGT |
| RAD21R3 | TACCTACTTATCTCCCTCTGAT |
| RAD21F2 | TGAAGGGTTCCTCGTATT |
| RAD21R2 | ATTTCCAGTCACTCTGTCTT |
| RAD21F1 | CTGATGCTTATTTGCCATTA |
| RAD21R1 | TTCCCCTCTTAGGTTTTCTT |
| RAD21PRO F1 | CTTTCTATCGCTTTGAATACA |
| RAD21PRO R1 | ACACAGAACCCTTTGAGAA |

Through deep sequencing, two novel SNPs were found. See Table 14. Methods of deep sequencing (or high-throughput comparitive sequence analysis) are described by Hartmer et al. (Nucleic Acids Res. May 1, 2003;31(9):e47), Bocker (Bioinformatics. July 2003;19 Suppl 1:I44-I153), in U.S. patent application 60/466,006 filed 25 Apr. 2003 and in U.S. patent application 60/429,895 filed 27 Nov. 2002.

TABLE 14

| | | Position in | | Allele |
|---|---|---|---|---|
| SNP ID | Chromosome | | Gene Position | Variants |
| RAD_11012 | 8 | 18828 | intron 5 del | —/CTTTAA |
| RAD_19951 | 8 | 27665 | Intron 2 | C/T |

Genotyping

There were two non-synomous proximal SNPs, two synomous proximal SNPs and one intronic SNP genotyped in the discovery cohort. The discovery cohort is described in Example 1. Three of the SNPs (rs1050838adj, rs1063547 and rs1804043) were found to be monomorphic by genotyping. One of the SNPs (rs1050838) is in particular significantly associated with breast cancer with a p-value of 0.0388. See Table 17. In addition, the two novel SNPs found through deep sequencing were genotyped in the discovery cohort. RAD_{—}11012 in particular showed a significant association with breast cancer with a p-value of 0.0005.

The methods used to verify and genotype the six proximal SNPs of Table 17 are the same methods described in Examples 1 and 2 herein. The PCR primers and extend primers used in these assays are provided in Table 15 and Table 16, respectively.

TABLE 15

| | | |
|---|---|---|
| dbSNP | Forward | Reverse |

| | | |
|---|---|---|
| rs# | PCR primer | PCR primer |

| | | |
|---|---|---|
| rs1050838 | ACGTTGGATGACCTCTTCCTCTTCATCATC | ACGTTGGATGACCAGAGTTAGAACTTCTGC |
| rs1050838adj | ACGTTGGATGTACTTACAGGCATCACAGGC | ACGTTGGATGAGATGAGTCAGCTATGCCTC |
| rs1063547 | ACGTTGGATGAGATGAGTCAGCTATGCCTC | ACGTTGGATGTACTTACAGGCATCACAGGC |
| rs3816342 | ACGTTGGATGATCACCACTTCAATGTTGGG | ACGTTGGATGCCTACTTCTCTCCCTATATG |
| rs1804043 | ACGTTGGATGTGTTCTCAGTAAAAGAGGGC | ACGTTGGATGACACATGGGCTTTGGTTAGC |
| RAD_11012 | ACGTTGGATGGAAGTCTTACTTCAAATGTT | ACGTTGGATGGAGTCATTTTAAAAAATTCAG |
| RAD_19951 | ACGTTGGATGATTGGAGTGCAAGGAAAATC | ACGTTGGATGCATATCAAGTCTATCTAGAGG |

TABLE 16

| | | |
|---|---|---|
| dbSNP | Extend | Term |

| | | |
|---|---|---|
| rs# | Primer | Mix |

| | | |
|---|---|---|
| rs1050838 | CTTCTGCCAGAAAAAGAGAAGGA | ACG |
| rs1050838adj | CTCAGGGAGTTAAGCGAAAAG | ACG |
| rs1063547 | ACAGGCTCTGGGTCAATTTGTCC | ACT |
| rs3816342 | TCTCTCCCTATATGCAATCA | ACG |
| rs1804043 | GCTTTGGTTAGCTTCTTATCC | ACC |
| RAD_11012 | ATTCAGATGCTAAAGAATT | CGT |
| RAD_19951 | TAGAGGTGATAAGGACTTCA | ACG |

Table 17, below, shows the case and control allele frequencies along with the p-values for all of the SNPs genotyped. The chromosome positions provided correspond to NCBI's build 31.

TABLE 17
Genotpying Results

| | Chromo- | | | Amino | | | |
|---|---|---|---|---|---|---|---|
| | some | Position in | | Acid | | | p- |
| Rs number | Position | | Alleles | Change | Case AF | Control AF | Value |
| rs1050838 | 117925391 | 13191 | G/A | A480A | G = 0.129 | G = 0.175 | 0.0388 |
| | | | | | A = 0.871 | A = 0.825 | |
| rs1050838adj | 117925390 | 13190 | G/A | G481R | monomorphic in genotyping | | |
| rs1063547 | 117924067 | 11867 | G/A | E528E | monomorphic in genotyping | | |
| rs3816342 | 117931549 | 19349 | G/A | Intron 5 | G = 0.129 | G = 0.164 | 0.1075 |
| | | | | | A = 0.871 | A = 0.836 | |
| rs1804043 | 117940075 | 27875 | A/G | Stop23W | monomorphic in genotyping | | |
| RAD_11012 | NOVEL | 18828 | —/ | Intron 5 | — = 0.151 | — = 0.081 | 0.0005 |
| | | | CTTTAA | | CTTTAA = | CTTTAA = | |
| | | | | | 0.849 | 0.919 | |
| RAD_19951 | NOVEL | 27665 | C/T | Intron 2 | C = 0.728 | C = 0.719 | 0.7615 |
| | | | | | T = 0.272 | T = 0.281 | |

Haplotype Analysis

Haplotype analysis was performed using four of the SNPs described in Table 17, along with the incident SNP (rs1374297). See Table 18. Haplotype frequencies, linkage disequilibrium statistics, and haplotype association tests are presented for each of the markers. Pairwise LD statistics estimated for rs3816342 and rs1374297 are highly significant (Table 18 and 19), indicating very strong LD between this pair of loci.

TABLE 18
Pairwise LD Statistics (r\hat{ }2)

| r2 | RAD_5376 | RAD_11012 | rs3816342 | RAD_19951 | rs1374297 |
|---|---|---|---|---|---|
| rs1050838 | 1 | 0.0186 | 0.971 | 0.067 | 0.0228 |
| RAD_11012 | 0.0186 | 1 | 0.0183 | 0.0494 | 0.982 |
| rs3816342 | 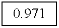 | 0.0183 | 1 | 0.066 | 0.0225 |
| RAD_19951 | 0.067 | 0.0494 | 0.066 | 1 | 0.0485 |
| rs1374297 | 0.0228 | 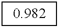 | 0.0225 | 0.0485 | 1 |

TABLE 19
Pairwise LD Statistics (D′)

| D′ | RAD_5376 | RAD_11012 | rs3816342 | RAD_19951 | rs1374297 |
|---|---|---|---|---|---|
| rs1050838 | 1 | 0.159 | 0.993 | 0.377 | 0.177 |
| RAD_11012 | 0.159 | 1 | 0.156 | 0.377 | 1 |
| rs3816342 | 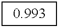 | 0.156 | 1 | 0.377 | 0.175 |
| RAD_19951 | 0.377 | 0.377 | 0.377 | 1 | 0.377 |
| rs1374297 | 0.177 | 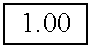 | 0.175 | 0.377 | 1 |

The frequencies of the haplotypes are provided in Table 20, and the association between haplotypes and breast cancer are provided in Table 21. As can be seen in Table 21, the haplotypes are in particular significantly associated with breast cancer (p-value=0.002191). The GWGCC haplotype is indicative of a low risk of breast cancer (i.e., a protective haplotype) whereas the other haplotypes in Table 21 are indicative of a higher risk of breast cancer. The general distribution of haplotypes among cases and controls is approximately the same in each subgroup, with the “AWACC” haplotype occurring at the highest frequency among cases. In the haplotypes, “M” represents the deleted allele, and “W” represents the CTTTAA allele of RAD_{—}1102.

TABLE 20
Haplotype frequencies

| | | |
|---|---|---|
| H | H freq | H rel freq |

| | | |
|---|---|---|
| AMACG | 124 | 0.114 |
| AWACC | 501 | 0.46 |
| AWATC | 298 | 0.274 |
| AWGCC | 1 | 0.001 |
| GMGCC | 2 | 0.002 |
| GWACC | 3 | 0.003 |
| GWGCC | 159 | 0.146 |

TABLE 21

| | | |
|---|---|---|
| Haplotype | Case | Control |

| | | |
|---|---|---|
| AMACG | 81 | 43 |
| AWACC | 243 | 258 |
| AWATC | 144 | 154 |
| AWGCC | 0 | 1 |
| GMGCC | 0 | 2 |
| GWACC | 0 | 3 |
| GWGCC | 70 | 89 |

Pearson's X2 = 20.5704; DF = 6; p-value = .002191

### Example 4

### RAD21 Expression Profile

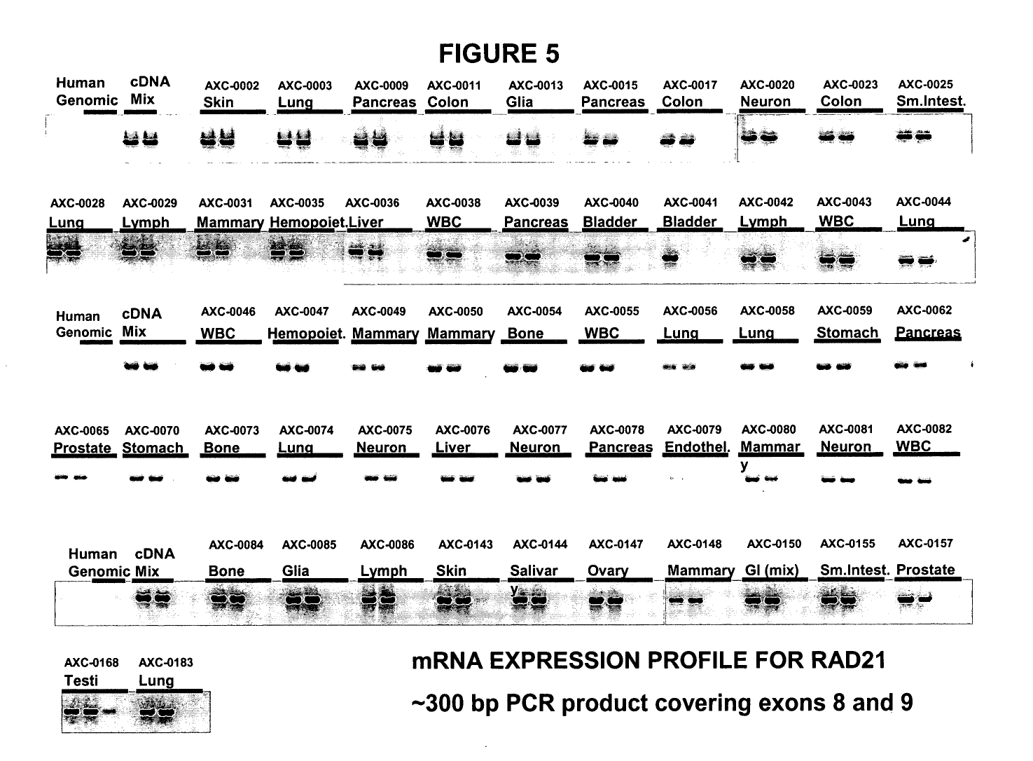

A cumulative mRNA expression profile was determined for RAD21 using a panel of 56 cells and tissues that represent a plurality of cells from different human tissue types. Specifically, RT-PCR was performed in cDNA made from 56 cell lines and 11 normal tissue samples using the following primers: forward, which spanned exons 8 and 9-CAATGCCAACCATGACTGAT and CGGTGTAAGACAGCGTGTAAA. The cDNA samples represent a variety of tissue types throughout the human body. The PCR reactions were done in a final volume of 10 μl using Hotstar Taq™ from Qiagen, Inc. Half of the PCR reaction was loaded on a 2% agarose gel to resolve the resulting product. From the expression profiling described above, RAD21 expression was found to be high and ubiquitous (see FIG. 5).

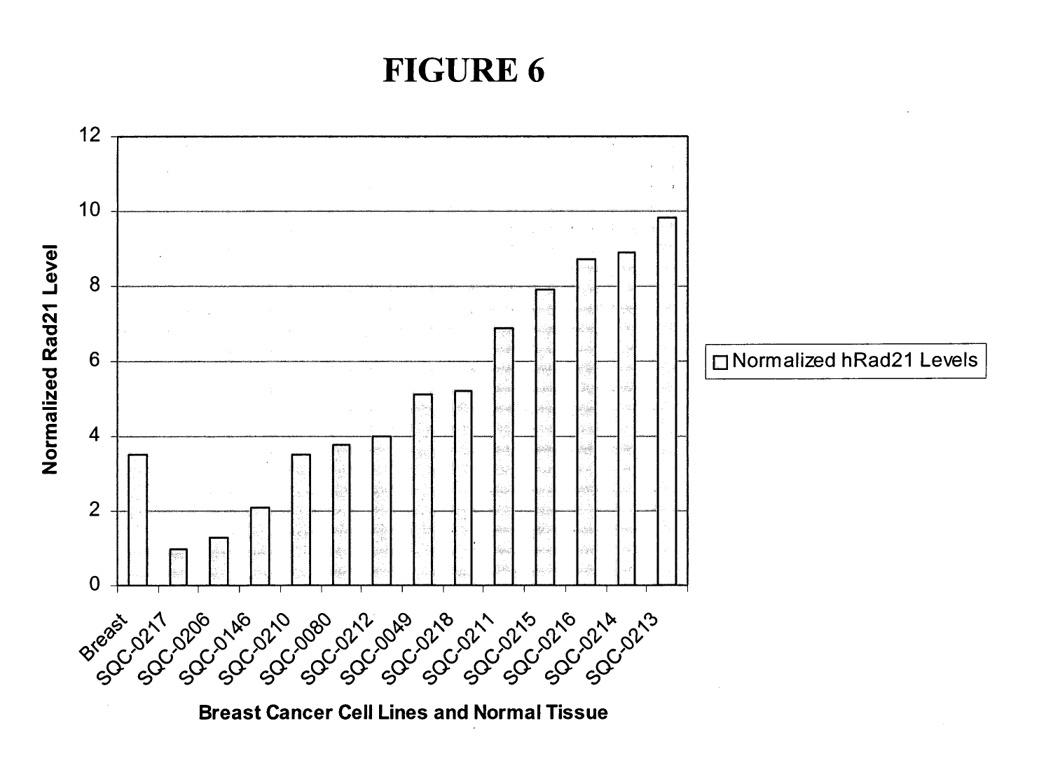

Quantitative RT-PCR hME was used to measure relative levels of RAD21 mRNA in several breast cancer cell lines and in a normal breast tissue (see FIG. 6). Total RNA isolated from 13 breast cancer cell lines and from normal tissue was reverse transcribed and quantified using competitor hME. Values were normalized to 18S rRNA. Normal (immortalized) breast cell lines, SQC-0217 and SQC-0206 were included.

Normalized RAD21 mRNA levels are lower in normal, immortalized breast cell lines, while higher levels are found in the tumorigenic breast cancer lines.

### Example 5

### Inhibition of RAD21 Gene Expression by Transfection of Specific siRNAs

RNAi-based gene inhibition was selected as a rapid way to inhibit expression of RAD21 in cultured cells. siRNA reagents were selectively designed to target RAD21. Algorithms useful for designing siRNA molecules specific for RAD21 are disclosed at the http address www.dhramacon.com. siRNA molecules up to 21 nucleotides in length were utilized. Table 23 summarizes the features of two duplexes that were used in the assays described herein. A non-homologous siRNA reagent (siRAD21_{—}1175S) was used as a negative control.

TABLE 23

| | |
|---|---|
| siRNA | SEQ |

| | | | |
|---|---|---|---|
| siRNA | Target | Sequence Specificity | ID NO: |

| | | |
|---|---|---|
| siRAD21_272 | RAD21 | AAGCCCAUGUGUUCGAGUGUA |
| siRAD21_1175 | RAD21 | AAGAGUUGGAUAGCAAGACAA |
| siRAD21_1175S | Non-homo- | AAGACAGAUACGAUGAUGAGA |
| | logous | |
| | scrambled | |
| | control | |

The siRNAs were transfected in cell lines MCF-7 and T-47D using Lipofectamine™ 2000 reagent from Invitrogen, Corp. 2.5 μg or 5.0 μg of siRNA was mixed with 6.25 μl or 12.5 μl lipofectamine, respectively, and the mixture was added to cells grown in 6-well plates. Their inhibitory effects on RAD21 gene expression were confirmed by precision expression analysis by MassARRAY (quantitativeRT-PCR hME), which was performed on RNA prepared from the transfected cells. See Chunming D. and Cantor C. PNAS 100(6):3059-3064 (2003). Cell viability was measured at 1, 2, 4 and 6 days post-transfection. Absorbance values were normalized relative to Day 1. RNA was extracted with Trizole reagent as recommended by the manufacturer (Invitrogen, Corp.) followed by cDNA synthesis using SuperScrip™ reverse transcriptase.

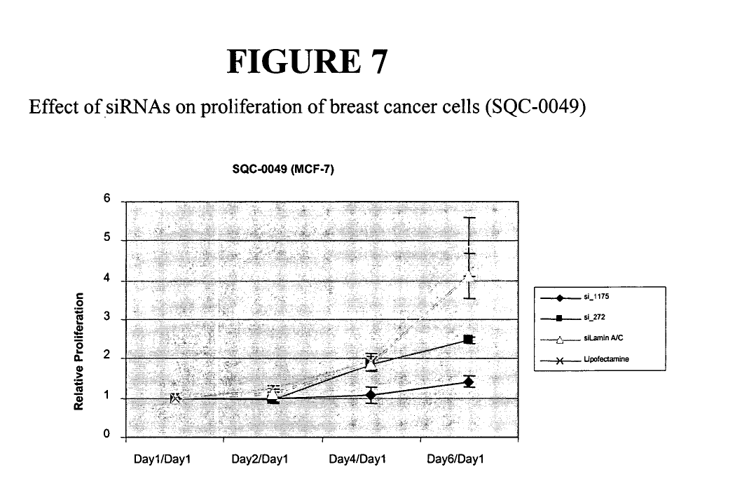

Strong inhibition of cell proliferation by siRAD21_{—}1175 is shown in FIG. 7, and the inhibitory effect of siRAD21_{—}272 is intermediate. These effects are consistent in all four experiments performed. The chart in FIG. 7 illustrates results of one representative experiment in MCF-7 cells. Each data point is an average of 3 wells of a 96-well plate normalized to values obtained from day 1 post transfection.

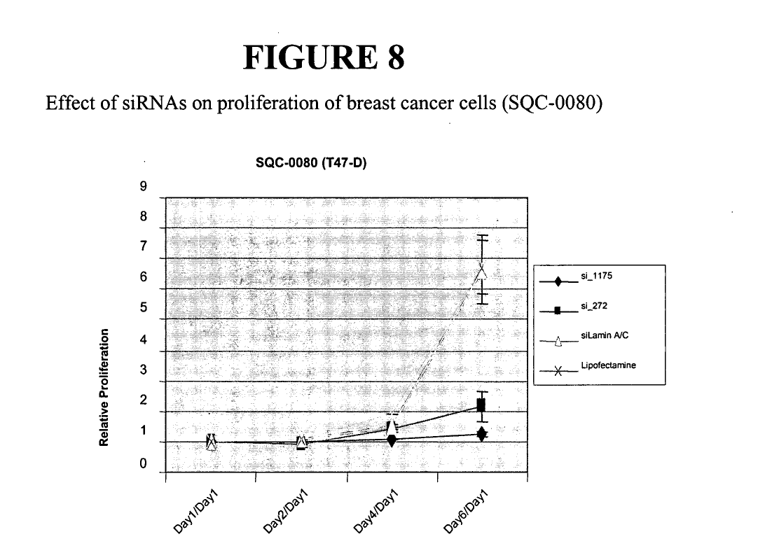

Similar to FIG. 7, siRAD21_{—}1175 showed strong inhibition of proliferation, and siRAD21_{—}272 showed intermediate effects on T47D cells in FIG. 8. These effects are consistent in all four experiments performed. The chart shows results of one representative experiment out of four. Each data point is an average of 3 wells of a 96-well plate normalized to values obtained from day 1 post transfection.

To confirm specificity of the active siRNAs, a scrambled control siRNA was designed based upon the sequence of the most active siRNA, siRAD21_{—}1175. The nucleotide sequence of the scrambled siRAD21_{—}1175S is composed of the same nucleotides as siRAD21_{—}1175 placed in a different order.

### Example 6

### Clonogenic Survival Assay

Clonogenic survival assays measure survival of cells after treatment with cytotoxic agents. In this particular assay the aim is to investigate the effect of downregulating RAD21 expression on sensitivity to therapeutically relevant DNA damaging agent. RAD21 is thought to be involved in the repair of DNA damage, often the repair of double strand breaks in DNA. When exposed to conditions that result in DSB, cells attempt to repair these damages. Defeciencies in mechanisms that repair these damages, such as a reduction in RAD21 expression, can result in unrepaired DNA breaks and eventual cell death. Due to the probable role of RAD21 in strand repair, it was hypothesized that cells are more sensitive to anti-cancer agents that cause strand breaks by attenuating RAD21 expression. This strategy is used to design therapies, which in combination with existing chemotherapeutic programs, increase sensitivity to DNA damaging/chemotherapeutic agents in the treatment of human malignancies.

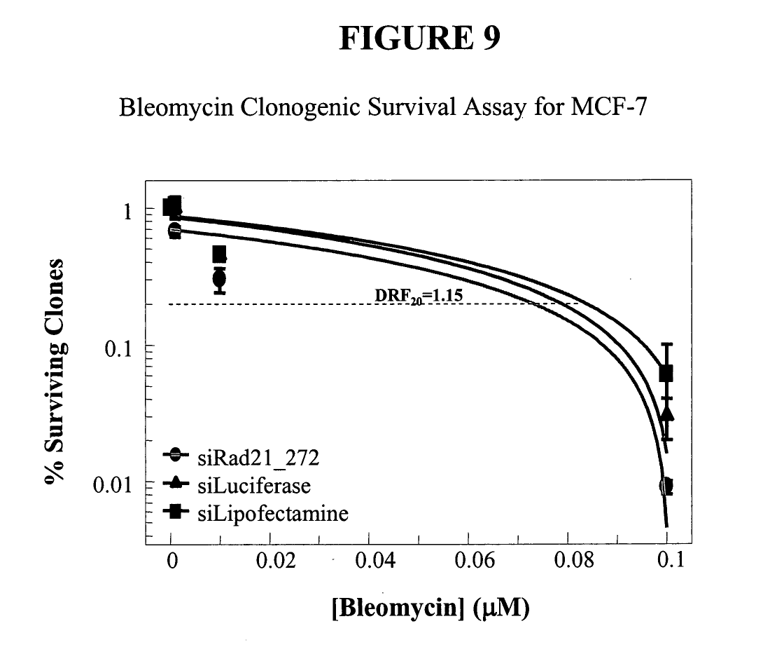

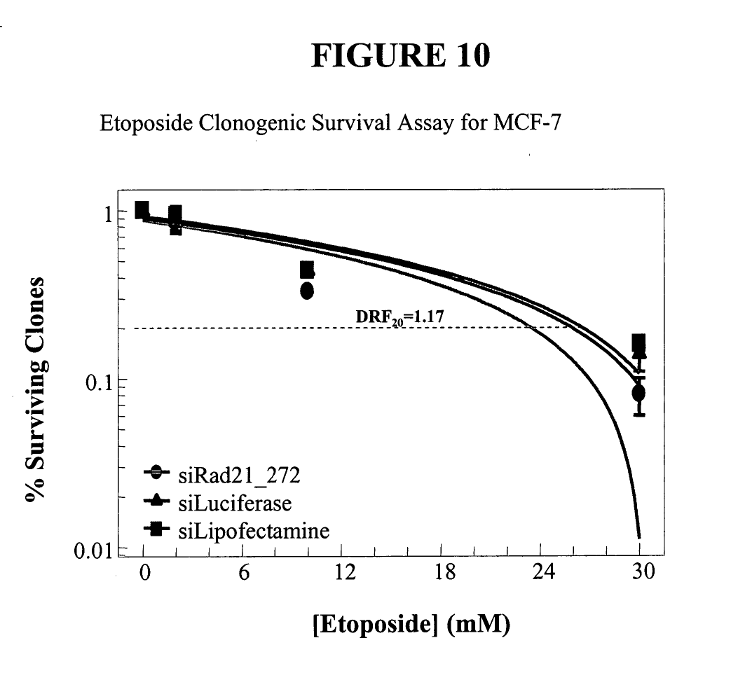

Cells were transfected with siRNA against RAD21, siRAD21_{—}272, to reduce expression of RAD21. Two days after transfection, the point of lowest expression of RAD21, cells were exposed to varying concentrations of bleomycin or etoposide (FIGS. 9 and 10, respectively) for 2 hours. Cells were then trypsinized and replated at 200 cells per plate. Cells were grown for 11-12 days, fixed and stained with crystal violet to detect colonies. Colonies were then counted. Survival was quantified as a fraction of the control results, cells transfected with unrelated siRNA, and plotted on a log scale as a function of dose. Increased sensitivity was measured as DRF, the factor by which the dose of radiation or drug can be reduced in the presence of the sensitizing agent, in this case decreased RAD21 expression to achieve the same level of cell killing in the absence of the sensitizing agent.

FIGS. 9 and 10 demonstrate an increased sensitivity to bleomycin and etoposide, respectively, in cell lines where RAD21 expression is inhibited using siRNA. The data were fitted to a curve and the DRF values calculated from the fitted curves of siRAD21 and Lipofectamine. The DRF20 for bleomycin is 1.15 and the DRF20 for etoposide is 1.17. These values are similar with values reported by Collis et al. in Cancer Res. Apr. 1, 2003;63(7):1550-4. In addition, these values are significant as can be seen from the error bars in FIGS. 9 and 10.

### Example 7

### Screening Assay to Detect Inhibitors of RAD21

The following is an exemplary assay for finding inhibitors of RAD21. There are many assays known in the art for detecting inhibitors. See e.g., Roth C et al. Genomics Feb. 1, 2003;63(3):384-90. Cells are transfected, transiently or often stably with the reporter construct described in Roth et al. Genomics Feb. 1, 2000;63(3):384-90. The cells often are chosen for minimal expression of endogenous RAD21, so that it can be externally introduced. Cells expressing the construct are co-transfected with a RAD21 expression vector or with vector lacking the RAD21 sequence (control). Both sets of cells are treated with the test compound and reporter gene activity is measured. Active compounds are selected based on their ability to prevent inhibition of the reporter gene expression when co-expressed with RAD21 and to have minimal effect on the control cells.

### Example 8

### In Vitro Production of RAD21 Polypeptides

cDNA is cloned into a pIVEX 2.3-MCS vector (Roche Biochem) using a directional cloning method. A cDNA insert is prepared using PCR with forward and reverse primers having 5′ restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of PCR products are cut with the corresponding restriction enzymes (i.e., Sal I and Sma I) and the products are gel-purified. The pIVEX 2.3-MCS vector is linearized using the same restriction enzymes, and the fragment with the correct sized fragment is isolated by gel-purification. Purified PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the RAD21 polypeptide for the purpose of protein purification. RAD21 polypeptide is purified by contacting the contents of reaction device with resin modified with Ni²⁺ ions. RAD21 polypeptide is eluted from the resin with a solution containing free Ni²⁺ ions.

### Example 9

### Cellular Production of RAD21 Polypeptides

Nucleic acids are cloned into DNA plasmids having phage recombination cites and RAD21 polypeptides are expressed therefrom in a variety of host cells. Alpha phage genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, Xis. DNA vectors have been produced where the integration/excision process is modified to allow for the directional integration or excision of a target DNA fragment into a backbone vector in a rapid in vitro reaction (Gateway™ Technology (Invitrogen, Inc.)).

A first step is to transfer the nucleic acid insert into a shuttle vector that contains attL sites surrounding the negative selection gene, ccdB (e.g. pENTER vector, Invitrogen, Inc.). This transfer process is accomplished by digesting the nucleic acid from a DNA vector used for sequencing, and to ligate it into the multicloning site of the shuttle vector, which will place it between the two attL sites while removing the negative selection gene ccdB. A second method is to amplify the nucleic acid by the polymerase chain reaction (PCR) with primers containing attB sites. The amplified fragment then is integrated into the shuttle vector using Int and IHF. A third method is to utilize a topoisomerase-mediated process, in which the nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the nucleic acid is transferred into the shuttle vector, it can be cloned into an expression vector having attR sites. Several vectors containing attR sites for expression of RAD21 polypeptide as a native polypeptide, N-fusion polypeptide, and C-fusion polypeptides are commercially available (e.g., pDEST (Invitrogen, Inc.)), and any vector can be converted into an expression vector for receiving a nucleic acid from the shuttle vector by introducing an insert having an attR site flanked by an antibiotic resistant gene for selection using the standard methods described above. Transfer of the nucleic acid from the shuttle vector is accomplished by directional recombination using hnt, IHF, and Xis (LR clonase). Then the desired sequence can be transferred to an expression vector by carrying out a one hour incubation at room temperature with Int, IHF, and Xis, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the RAD21 polypeptide or polypeptide variants. These expression vectors are often transfected, for example, into murine-transformed a adipocyte cell line 3T3-L1, (ATCC), human embryonic kidney cell line 293, and rat cardiomyocyte cell line H9C2.

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow.

Citation of the above publications or documents is not intended as an admission that any of the foregoing is pertinent prior art, nor does it constitute any admission as to the contents or date of these publications or documents. U.S. patents and other publications referenced herein are hereby incorporated by reference.

1. A method for identifying a subject at risk of breast cancer, which comprises detecting the presence or absence of one or more polymorphic variations associated with breast cancer in a nucleic acid sample from a subject, wherein the one or more polymorphic variations are detected in a nucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c);
whereby the presence of the polymorphic variation is indicative of the subject being at risk of breast cancer.

2. The method of claim 1, which further comprises obtaining the nucleic acid sample from the subject.

3. The method of claim 1, wherein the one or more polymorphic variations are detected at one or more positions in SEQ ID NO: 1 is selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

4. The method of claim 1, wherein the one or more polymorphic variations are detected within a region spanning positions 13191 to 33670 in SEQ ID NO: 1.

5. The method of claim 3, wherein a polymorphic variation is detected at position 18828 in SEQ ID NO: 1.

6. The method of claim 3, wherein a polymorphic variation is detected at position 13191 in SEQ ID NO: 1.

7. The method of claim 3, wherein a polymorphic variation is detected at position 33454 and/or 33670 in SEQ ID NO: 1.

8. The method of claim 1, wherein the one or more polymorphic variations are detected at one or more positions in linkage disequilibrium with one or more positions in SEQ ID NO: 1 selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

9. The method of claim 1, wherein detecting the presence or absence of the one or more polymorphic variations comprises:
hybridizing an oligonucleotide to the nucleic acid sample, wherein the oligonucleotide is complementary to a nucleotide sequence in the nucleic acid and hybridizes to a region adjacent to the polymorphic variation;
extending the oligonucleotide in the presence of one or more nucleotides, yielding extension products; and
detecting the presence or absence of a polymorphic variation in the extension products.

10. The method of claim 1, wherein the subject is a human.

11. A method for identifying a polymorphic variation associated with breast cancer proximal to an incident polymorphic variation associated with breast cancer, which comprises:
identifying a polymorphic variation proximal to the incident polymorphic variation associated with breast cancer, wherein the polymorphic variation is detected in a nucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c) comprising the polymorphic variation;
determining the presence or absence of an association of the proximal polymorphic variant with breast cancer.

12. The method of claim 11, wherein the incident polymorphic variation is at a position in SEQ ID NO: 1 selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

13. The method of claim 1, wherein the incident polymorphic variation is within a region spanning positions 13191 to 33670 in SEQ ID NO: 1.

14. The method of claim 11, wherein the proximal polymorphic variation is within a region between about 5 kb 5′ of the incident polymorphic variation and about 5 kb 3′ of the incident polymorphic variation.

15. The method of claim 11, which further comprises determining whether the proximal polymorphic variation is in linkage disequilibrium with the incident polymorphic variation.

16. The method of claim 11, which further comprises identifying a second polymorphic variation proximal to the identified proximal polymorphic variation associated with breast cancer and determining if the second proximal polymorphic variation is associated with breast cancer.

17. The method of claim 16, wherein the second proximal polymorphic variant is within a region between about 5 kb 5′ of the incident polymorphic variation and about 5 kb 3′ of the proximal polymorphic variation associated with breast cancer.

18. An isolated nucleic acid comprising a nucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c); and
(e) a nucleotide sequence complementary to the nucleotide sequences of (a), (b), (c), or (d);
wherein the nucleotide sequence comprises a variation at a position in FIGS. 1A-1S associated with breast cancer selected from the group consisting of a guanine at position 17237, a guanine at position 33545, a guanine at position 33670, an adenine at position 13191 and a CTTTAA deletion at position 18828.

19. An oligonucleotide comprising a nucleotide sequence complementary to a portion of the nucleotide sequence of (a), (b), (c), or (d) in claim 18, wherein the 3′ end of the oligonucleotide is adjacent to a polymorphic variation associated with breast cancer.

20. A microarray comprising an isolated nucleic acid of claim 18 linked to a solid support.

21. An isolated polypeptide encoded by the isolated nucleic acid sequence of claim 18.

22. A method for identifying a candidate molecule that modulates cell proliferation, which comprises:
(a) introducing a test molecule to a system which comprises a nucleic acid comprising a nucleotide sequence selected from the group consisting of:
(i) a nucleotide sequence in FIGS. 1A-1S or FIG. 2;
(ii) a nucleotide sequence which encodes a polypeptide encoded by a nucleotide sequence in FIGS. 1A-1S or FIG. 2;
(iii) a nucleotide sequence which encodes a polypeptide that is 90% or more identical to the amino acid sequence encoded by a nucleotide sequence in FIGS. 1A-1S or FIG. 2;
(iv) a fragment of a nucleotide sequence of (i), (ii), or (iii); or
introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate molecule that modulates cell proliferation.

23. The method of claim 22, wherein the system is an animal.

24. The method of claim 22, wherein the system is a cell.

25. The method of claim 22, wherein the nucleotide sequence comprises one or more polymorphic variations associated with breast cancer.

26. The method of claim 25, wherein the one or more polymorphic variations associated with breast cancer are at one or more positions in SEQ ID NO: 1 is selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

27. The method of claim 25, wherein the one or more polymorphic variations associated with breast cancer are in a region spanning positions 13191 to 33670 in SEQ ID NO: 1.

28. A method for treating breast cancer in a subject, which comprises administering a candidate molecule identified by the method of claim 23 to a subject in need thereof, whereby the candidate molecule treats breast cancer in the subject.

29. A method for identifying a candidate therapeutic for treating breast cancer, which comprises:
(a) introducing a test molecule to a system which comprises a nucleic acid comprising a nucleotide sequence selected from the group consisting of:
(i) a nucleotide sequence in FIGS. 1A-1S or FIG. 2;
(ii) a nucleotide sequence which encodes a polypeptide encoded by a nucleotide sequence in FIGS. 1A-1S or FIG. 2;
(iii) a nucleotide sequence which encodes a polypeptide that is 90% or more identical to the amino acid sequence encoded by a nucleotide sequence in FIGS. 1A-1S or FIG. 2;
(iv) a fragment of a nucleotide sequence of (i), (ii), or (iii); or
introducing a test molecule to a system which comprises a protein encoded by a nucleotide sequence of (i), (ii), (iii), or (iv); and
(b) determining the presence or absence of an interaction between the test molecule and the nucleic acid or protein,
whereby the presence of an interaction between the test molecule and the nucleic acid or protein identifies the test molecule as a candidate therapeutic for treating breast cancer.

30. A method for treating breast cancer in a subject, which comprises contacting one or more cells of a subject in need thereof with a nucleic acid, wherein the nucleic acid comprises a nucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c); and
(e) a nucleotide sequence complementary to the nucleotide sequences of (a), (b), (c), or (d);
whereby contacting the one or more cells of the subject with the nucleic acid treats breast cancer in the subject.

31. The method of claim 30, wherein the nucleic acid is RNA or PNA.

32. The method of claim 31, wherein the nucleic acid is duplex RNA.

33. The method of claim 32, wherein a strand of the duplex RNA comprises the nucleotide sequence AAGCCCAUGUGUUCGAGUGUA (SEQ ID NO: 232) or AAGAGUUGGAUAGCAAGACAA (SEQ ID NO: 233).

34. A method for treating breast cancer in a subject, which comprises contacting one or more cells of a subject in need thereof with a protein, wherein the protein is encoded by a nucleotide sequence which comprises a polynucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c);
whereby contacting the one or more cells of the subject with the protein treats breast cancer in the subject.

35. A method for treating breast cancer in a subject, which comprises:
detecting the presence or absence of one or more polymorphic variations associated with breast cancer in a nucleic acid sample from a subject, wherein the one or more polymorphic variation are detected in a nucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c) comprising the polymorphic variation; and
administering a breast cancer treatment to a subject in need thereof based upon the presence or absence of the one or more polymorphic variations in the nucleic acid sample.

36. The method of claim 35, wherein the one or more polymorphic variations are at one or more positions in SEQ ID NO: 1 selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

37. The method of claim 36, wherein the one or more polymorphic variations are in a region spanning positions 13191 to 33670 in SEQ ID NO: 1.

38. The method of claim 35, which further comprises extracting and analyzing a tissue biopsy sample from the subject.

39. The method of claim 35, wherein the treatment is chemotherapy, surgery, radiation therapy, a process that induces nucleic acid strand breakage, and combinations of the foregoing.

40. The method of claim 39, wherein the chemotherapy is selected from the group consisting of cyclophosphamide (Cytoxan), methotrexate (Amethopterin, Mexate, Folex), fluorouracil (Fluorouracil, 5-Fu, Adrucil), cyclophosphamide, doxorubicin (Adriamycin), and combinations of the foregoing.

41. The method of claim 40, wherein the combinations are selected from the group consisting of cyclophosphamide (Cytoxan), methotrexate (Amethopterin, Mexate, Folex), and fluorouracil (Fluorouracil, 5-Fu, Adrucil); cyclophosphamide, doxorubicin (Adriamycin), and fluorouracil; and doxorubicin and cyclophosphamide.

42. A method for detecting or preventing breast cancer in a subject, which comprises:
detecting the presence or absence of one or more polymorphic variations associated with breast cancer in a nucleic acid sample from a subject, wherein the polymorphic variation is detected in a nucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c) comprising the polymorphic variation; and
administering a breast cancer preventative or detection procedure to a subject in need thereof based upon the presence or absence of the one or more polymorphic variations in the nucleic acid sample.

43. The method of claim 42, wherein the one or more polymorphic variations are at one or more positions in SEQ ID NO: 1 is selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 833242.

44. The method of claim 43, wherein the one or more polymorphic variations are in a region spanning positions 13191 to 33670 in SEQ ID NO: 1.

45. The method of claim 42, wherein the breast cancer detection procedure is selected from the group consisting of a mammography, an early mammography program, a frequent mammography program, a biopsy procedure, a breast biopsy and biopsy from another tissue, a breast ultrasound and optionally ultrasound analysis of another tissue, breast magnetic resonance imaging (MRI) and optionally MRI analysis of another tissue, electrical impedance (T-scan) analysis of breast and optionally of another tissue, ductal lavage, nuclear medicine analysis (e.g., scintimammography), BRCA1 and/or BRCA2 sequence analysis results, thermal imaging of the breast and optionally of another tissue, and a combination of the foregoing.

46. The method of claim 42, wherein the breast cancer preventative procedure is selected from the group consisting of one or more selective hormone receptor modulators, one or more compositions that prevent production of hormones, one or more hormonal treatments, one or more biologic response modifiers, surgery, and drugs that delay or halt metastasis.

47. The method of claim 46, wherein the selective hormone receptor modulator is selected from the group consisting of tamoxifen, reloxifene, and toremifene; the composition that prevents production of hormones is an aramotase inhibitor selected from the group consisting of exemestane, letrozole, anastrozol, groserelin, and megestrol; the hormonal treatment is selected from the group consisting of goserelin acetate and fulvestrant; the biologic response modifier is an antibody that specifically binds herceptin/HER2; the surgery is selected from the group consisting of lumpectomy and mastectomy; and the drug that delays or halts metastasis is pamidronate disodium.

48. A method of targeting information for preventing or treating breast cancer to a subject in need thereof, which comprises:
detecting the presence or absence of one or more polymorphic variations associated with breast cancer in a nucleic acid sample from a subject, wherein the polymorphic variation is detected in a nucleotide sequence selected from the group consisting of:
(a) a nucleotide sequence in FIGS. 1A-1S or FIG. 2;
(b) a nucleotide sequence which encodes a polypeptide encoded by a nucleotide sequence in FIGS. 1A-1S or FIG. 2;
(c) a nucleotide sequence which encodes a polypeptide that is 90% or more identical to the amino acid sequence encoded by a nucleotide sequence in FIGS. 1A-1S or FIG. 2;
(d) a fragment of a nucleotide sequence of (a), (b), or (c) comprising the polymorphic variation; and
directing information for preventing or treating breast cancer to a subject in need thereof based upon the presence or absence of the one or more polymorphic variations in the nucleic acid sample.

49. The method of claim 48, wherein the one or more polymorphic variations are at one or more positions in SEQ ID NO: 1 is selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

50. The method of claim 48, wherein the one or more polymorphic variations are in a region spanning positions 13191 to 33670 in SEQ ID NO: 1.

51. The method of claim 48, wherein the information comprises a description of a breast cancer detection procedure, a chemotherapeutic treatment, a surgical treatment, a radiation treatment, a preventative treatment of breast cancer, and combinations of the foregoing.

52. A composition comprising a breast cancer cell and an antibody that specifically binds to a protein, polypeptide or peptide encoded by a nucleotide sequence identical to or 90% or more identical to a nucleotide sequence in FIGS. 1A-1S or FIG. 2.

53. A composition comprising a breast cancer cell and a RNA, DNA, PNA or ribozyme molecule comprising a nucleotide sequence identical to or 90% or more identical to a portion of a nucleotide sequence in FIGS. 1A-1S or FIG. 2.

54. The composition of claim 53, wherein the RNA molecule is a short inhibitory RNA molecule.

55. A composition comprising a breast cancer cell and an agent that induces nucleic acid strand breakage.

56. A method of selecting a subject that will respond to a treatment of breast cancer, which comprises:
detecting the presence or absence of one or more polymorphic variations associated with breast cancer in a nucleic acid sample from a subject, wherein the polymorphic variation is detected in a nucleotide sequence selected from the group consisting of:
(a) the nucleotide sequence of FIGS. 1A-1S or FIG. 2;
(b) a nucleotide sequence which encodes a polypeptide consisting of an amino acid sequence encoded by a nucleotide sequence in FIGS. 1A-1S or FIG. 2;
(c) a nucleotide sequence which encodes a polypeptide that is 90% or more identical to an amino acid sequence encoded by a nucleotide sequence in FIGS. 1A-1S or FIG. 2; and
(d) a fragment of a nucleotide sequence of (a), (b), or (c) comprising the polymorphic variation; and
selecting a subject that will respond to the breast cancer treatment based upon the presence or absence of the one or more polymorphic variations in the nucleic acid sample.

57. The method of claim 56, wherein the one or more polymorphic-variations are detected at one or more positions in SEQ ID NO: 1 selected from the group consisting of 56, 7324, 7363, 9231, 10490, 11867, 12308, 13191, 13525, 13582, 15875, 17237, 18472, 19170, 19349, 23869, 24235, 26691, 31373, 31979, 33472, 33545, 33670, 33778, 34691, 36859, 47651, 48463, 49058, 50233, 51228, 52315, 53039, 62940, 64531, 64989, 65209, 65444, 70056, 70329, 70629, 71326, 72563, 73135, 73627, 74621, 75303, 75749, 75855, 77799, 78432, 78648, 79585, 79791, 80037, 80082, 82490 and 83324.

58. The method of claim 56, wherein the one or more polymorphic variation are detected in a region spanning positions 13191 to 33670 in SEQ ID NO: 1.

59. The method of claim 56, wherein the treatment induces nucleic acid strand breakage.
